(19) United States
(12) Patent Application Publication
(10) Pub. No.: US 2025/0144119 A1
(43) Pub. Date: May 8, 2025

(54) ACTIVATING ENDOGENOUS ANTIMICROBIALS TO TREAT SARS-COV-2 INFECTION

(71) Applicant: Eirgen Pharma Ltd., Waterford (IE)

(72) Inventors: Charles W. Bishop, Maimi, FL (US); Stephen A. Strugnell, Madison, WI (US); Akhtar Ashfaq, Westbury, NY (US); Reem Elamein Elsiddig, Waterford (IE); Colm Nulty, Waterford (IE)

(21) Appl. No.: 18/744,352

(22) Filed: Jun. 14, 2024

Related U.S. Application Data
(63) Continuation of application No. 17/223,631, filed on Apr. 6, 2021, now abandoned.
(60) Provisional application No. 63/032,714, filed on May 31, 2020.
(60) Provisional application No. 63/012,781, filed on Apr. 20, 2020.
(60) Provisional application No. 63/009,155, filed on Apr. 13, 2020.
(60) Provisional application No. 63/006,563, filed on Apr. 7, 2020.
(60) Provisional application No. 63/006,034, filed on Apr. 6, 2020.

(51) Int. Cl. A61K 31/593; A61K 9/00; A61P 31/14

(52) U.S. Cl. CPC A61K 31/593; A61K 9/0053; A61P 31/14

(57) ABSTRACT

Provided herein are methods of treating COVID-19 in a subject in need thereof, comprising administering to the subject a 25-hydroxyvitamin D compound. Also provided herein are hard capsule dosage forms of 25-hydroxyvitamin. In aspects, the 25-hydroxyvitamin D is administered as a controlled release formulation, optionally an extended release oral formulation, such as Rayaldee® extended release calcifediol capsules. Methods of treating SARS-CoV-2 infection including reducing SARS-CoV-2 viral load are provided. Methods of treating SARS-CoV-2 infection including increasing an immune response are provided.

### CROSS-REFERENCE TO RELATED APPLICATIONS

This application is a Continuation of U.S. application Ser. No. 17/223,631, filed Apr. 6, 2021, which claims the benefit under 35 U.S.C. § 119(e) of U.S. Provisional Patent Application Nos. 63/006,034, filed Apr. 6, 2020, 63/006,563, filed Apr. 7, 2020, 63/009,155, filed Apr. 13, 2020, 63/012,781, filed Apr. 20, 2020, and 63/032,714, filed May 31, 2020, is hereby claimed, and the disclosures thereof are hereby incorporated by reference herein.

### INCORPORATION BY REFERENCE OF SEQUENCE LISTING

Incorporated by reference in its entirety is a computer-readable nucleotide/amino acid sequence listing submitted concurrently herewith and identified as follows: ASCII (text) file named 55530F SeqListing.xml; size: 31,030 bytes, created Jun. 14, 2024.

### BACKGROUND

### Field of the Disclosure

The disclosure relates generally to treatment of SARS-CoV-2 infection. More particularly, the disclosure relates to treatment of SARS-CoV-2 infection with calcifediol, including extended release calcifediol (ERC), and characterized by a target serum total 25-hydroxyvitamin D concentration threshold.

### Brief Description of Related Technology

The Severe Acute Respiratory Syndrome Coronavirus 2 (SARS-CoV-2) is a novel virus belonging to the Coronaviridae family of enveloped viruses. SARS-CoV-2 is a positive-sense single-stranded RNA virus with genetic similarity to bat coronaviruses and was first isolated in January 2020 from patients in Wuhan, China (Hui et al., International J Infectious Diseases 91: 264-266 (2020). As of the end of March 2020, over 900,000 people were infected by SARS-CoV-2 and over 45,000 people died from Coronavirus Disease 2019 (COVID-19), the disease caused by SARS-CoV-2 infection. By the end of May 2020, almost six million people are diagnosed with an infection with over three hundred fifty thousand deaths worldwide.

Currently, there are no therapeutic agents approved by a government regulatory agency for the treatment of COVID-19 or for SARS-CoV-2 infection. Though antimalarial agents, chloroquine and hydroxychloroquine, with or without an antibiotic (e.g., azithromycin), have been evaluated for treating SARS-CoV-2 patients (Liu et al., Cell Discov 6:16 (2020); Yao et al., Clin Infect Dis PMID 32150618; and Gautret et al., Int J Antimicro Agents PMID 32205204, 105949 (2020)), the therapeutic efficacy has yet to be demonstrated in a large, properly controlled, clinical trial setting. See, e.g., Yazdany and Kim, Annals of Internal Medicine DOI: 10.7326/M20-1334 (2020). Thus, there is a need for methods of preventing infection with SARS-CoV-2 and treating subjects infected with SARS-CoV-2 or subjects with COVID-19.

Extended release calcifediol (ERC) dosage forms and related methods have been described, e.g. in U.S. Pat. Nos. 8,2207,149, 8,426,391, 9,861,644, and U.S. Patent Application Publication No. 2019/0083513 A1, and international patent application publication WO 2020/044314 A1, the entire disclosures of which are incorporated herein by reference. A related FDA-approved product is marketed as Rayaldee® calcifediol extended-release capsules.

### SUMMARY

One aspect of the disclosure provides a method for treating or preventing a SARS-CoV-2 infection or COVID-19 disease comprising administering to a subject in need thereof an effective amount 25-hydroxyvitamin D, optionally a sustained release formulation thereof to achieve a serum total 25-hydroxyvitamin D level of at least 50 ng/mL, or at least 60 ng/mL.

Another aspect of the disclosure provides a method of decreasing viral load of a subject in need thereof and infected with SARS-CoV-2, comprising administering to a subject in need thereof an effective amount 25-hydroxyvitamin D, optionally a sustained release formulation thereof to achieve a serum total 25-hydroxyvitamin D level of at least 50 ng/mL, or at least 60 ng/mL.

Another aspect of the disclosure provides 25-hydroxyvitamin D composition for use in a method of treating a SARS-CoV-2 infection or COVID-19 disease, optionally an extended release 25-hydroxyvitamin D composition, as further described herein.

Another aspect of the disclosure provides a use of 25-hydroxyvitamin D in the manufacture of a medicament to treat a SARS-CoV-2 infection or COVID-19 disease, optionally a sustained release formulation of 25-hydroxyvitamin D, as further described herein.

Another aspect of the disclosure provides a soft capsule controlled release dosage form comprising 25-hydroxyvitamin D and a wax-based controlled release agent, wherein the in vitro and in vivo release profile of the dosage form is faster than the reference dosage form RAYALDEE, optionally 1-10% faster.

Another aspect of the disclosure provides a method of treating a condition associated with a SARS-CoV-2 infection in a patient, comprising administering to the patient a pharmaceutically effective amount of a maintenance dose of 25-hydroxyvitamin D3 in an oral dosage form wherein the patient's vitamin D metabolite ratio (VMR, calculated as 100 times the ratio of serum 24,25-dihydroxoxyvitamin D3 to serum 25-hydroxyvitamin D3) remains substantially constant or decreases throughout the maintenance dosing period.

Another aspect of the disclosure provides a dosing regimen for treating a patient having or suspected of having a SARS-CoV-2 infection, comprising administering to the patient an extended release dosage form comprising a controlled release excipient and a pharmaceutically effective amount of 25-hydroxyvitamin D₃ and wherein the dosage form is administered in a maintenance dosing period in an amount of 30 to 70 μg/day and optionally preceded by a loading dosing period in an amount in a range of 300 to 900 μg/day on days one, two or three of treatment, wherein the patient's vitamin D metabolite ratio (VMR, calculated as 100 times the ratio of serum 24,25-dihydroxoxyvitamin D3 to serum 25-hydroxyvitamin D₃) remains substantially constant or decreases during the maintenance dosing period.

Another aspect of the disclosure provides a hard capsule dosage form comprising a hard shell capsule containing a solid or semi-solid composition comprising a 25-hydroxyvitamin D compound.

For the compositions and methods described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the methods, uses, and compositions are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

To further facilitate the understanding of the present invention, ten figures are attached hereto.

FIG. 1 shows projected serum total 25-hydroxyvitamin D levels as a function of dosing adults with immediate release 25-hydroxyvitamin D₃ (bioavailability about 75%), by dosing with 532 mcg on Day 1 and 266 mcg on Days 3, 7, 14, 21 and 28, assuming a baseline serum total 25-hydroxyvitamin D level of 25 ng/mL.

FIGS. 2-4 show modeled serum 25-hydroxyvitamin D₃ levels for subjects according to the dosing method described in Example 4, in subjects who follow dosing when fasted (FIG. 2), subjects who administer their doses with food (FIG. 3), and subjects who administer their doses with an average of fed and fasted dosing (FIG. 4).

FIGS. 5 and 6 show frequency distributions of subjects who have returned to usual activities and returned to normal health according to Example 4.

FIG. 7 shows mean serum concentration of 25-hydroxyvitamin D₃ curves after oral administration of 900 mcg of modified release calcifediol capsules.

FIG. 8 shows in vitro dissolution profiles of dosage forms according to the disclosure.

FIG. 9 shows serum total 25-D concentrations as a function of time with repeated dosing of ERC (Rayaldee®-type formulation), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency.

FIG. 10 shows VMR as a function of time with repeated dosing of ERC (Rayaldee®-type formulation), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency.

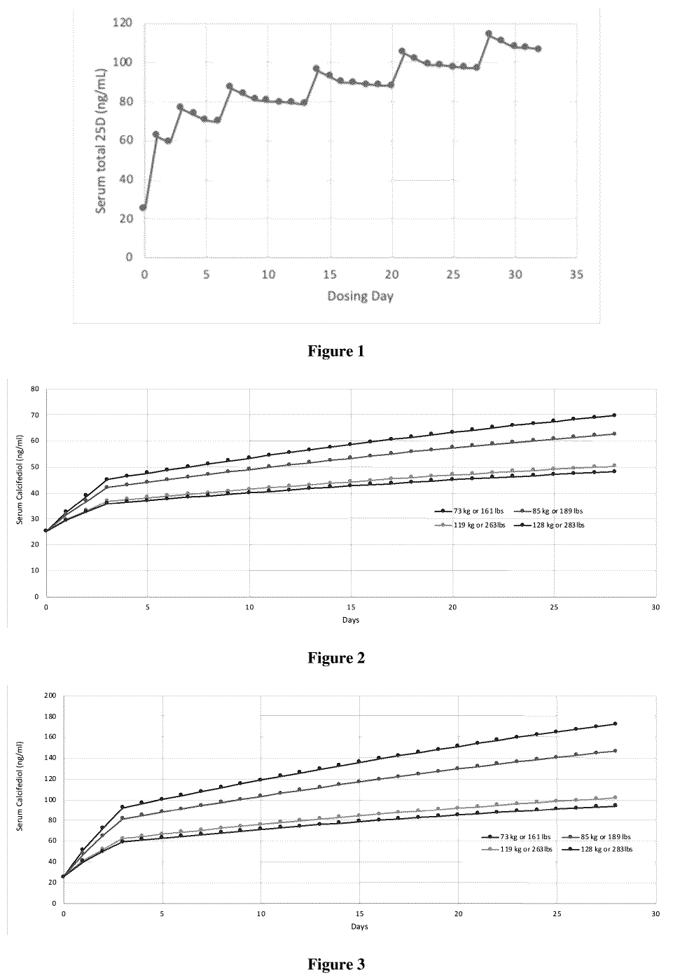

### DESCRIPTION

The innate immune response in antigen-presenting cells (monocyte/macrophages and dendritic cells) is initiated and perpetuated by pathogen-associated molecular patterns (PAMPs) of the infecting virus interacting with pattern recognition receptors (e.g., Toll-like receptors [TLR]). TLR activation leads to up-regulation of expression of the intracellular vitamin D receptor (VDR) and CYP27B1 (25-hydroxyvitamin D [25D]-1α-hydroxylase); the latter event up-regulates local generation of the active vitamin D metabolite, 1,25-dihydroxyvitamin D. 1,25-dihydroxyvitamin D can then engage the VDR in an intracrine mode, which, in turn, controls Cathelicidin antimicrobial peptide (CAMP) and interleukin 1β (IL-1β) gene expression, IL-1β being a central initiator of the adaptive immune response. When 25-hydroxyvitamin D is supplied in sufficient quantities to macrophages, the cells locally produce 1,25-dihydroxyvitamin D, which binds to the vitamin D receptor (VDR). The activation of VDR in immune cells, like macrophages, leads to an upregulation of the Cathelicidin Antimicrobial Peptide (CAMP) gene due to the presence of a vitamin D responsive element in the promoter of this gene. Expression of CAMP leads to an increased production of a precursor peptide, called CAP-18, the cleavage of which leads to the production of two antimicrobial peptides (AMPs), termed LL37 and FALL 39.

Enzymatic production of 1,25-dihydroxyvitamin D is dependent on the serum supply of substrate 25-hydroxyvitamin D. 25-hydroyxvitamin D is the major circulating metabolite of vitamin D in man. Expression and secretion of LL37, along with numerous other endogenous antimicrobial activities, requires induction and priming. A key regulatory signal that can prime human macrophages for rapid and robust antimicrobial responses, including deployment of LL37 at the site of infection, is vitamin D, specifically 25-hydroxyvitamin D (Gombart A F, Saito T, Koeffler H P. Exaptation of an ancient Alu short interspersed element provides a highly conserved vitamin D-mediated innate immune response in humans and primates. BMC Genomics. 2009; 10:321).

Macrophages without sufficient serum total 25-hydroxyvitamin D are rendered unable to release LL37, and thus protect the host. An insufficient level of total 25-hydroxyvitamin D in human serum prohibits i) adequate intracellular generation of 1,25D, ii) adequate activation of the VDR, iii) adequate transactivation of the Cathelicidin gene and iv) adequate production and release of LL37 to combat microbial invasion of the host. The end result is innate and subsequent adaptive immune deficiency.

Without intending to be bound by any particular theory, it is believed that raising serum total 25-hydroxyvitamin D to sufficiently high levels can resolve the immune-compromised state in the human host and avoid worsening of progression of the infection and related complications. From another perspective, it is believed that raising serum total 25-hydroxyvitamin D to sufficiently high levels can prevent an immune-compromised state, or a more severe immune-compromised state, in the human host and avoid infection or worsening of progression of the infection and related complications. Accomplishing this with vitamin D supplements is unreliable and is difficult in obesity owing to the increased volume of distribution of the non-polar vitamin. According to the latest reported United States data, many SARS-CoV-2 subjects are expected to have higher than normal body mass index (BMI). In contrast, raising 25-hydroxyvitamin D and/or ERC is more rapid, reliable and, depending on the administered dose, can take as little as 4, 8 or 9 to 12 hours. Treatment with 25-hydroxyvitamin D and has been shown to be safe. Treatment with ERC has shown to be safe for administration to patients with Stage 3 or 4 Chronic Kidney Disease (CKD) as evidence by US FDA approval of Rayaldee® (calcifediol) Extended-release Capsules. Rayaldee® (calcifediol) Extended-release Capsules, was approved by the US Food and Drug Administration (FDA) in 2016 to treat secondary hyperparathyroidism (SHPT) in adult patients with stage 3 or 4 CKD by raising serum total 25-hydroxyvitamin D from <30 ng/mL to the range of 30 to 100 ng/mL. Serum total 25-hydroxyvitamin D levels of ≥50 ng/mL have recently been shown to be required for 25-hydroxyvitamin D to produce vitamin D-responsive endpoints in patients with chronic kidney disease (CKD), probably via activation by extra-renal CYP27B1 (Strugnell S A, Sprague S M, Ashfaq A et al. Rationale for Raising Current Clinical Practice Guideline Target for Serum 25-Hydroxyvitamin D in Chronic Kidney Disease. Am J Nephrol. 2019; 49:284-293). Without intending to be bound by any particular theory, it appears likely that similar levels, inducing extra-renal activation of 25-hydroxyvitamin D, are required for 25-hydroxyvitamin D to be activated in vivo by CYP27B1 expressed in virus-stimulated macrophages. Furthermore, safe and effective achievement of serum total 25-hydroxyvitamin D levels at least 50 ng/mL (with related rises over time in 1,25-dihydroxyvitamin D) have been shown with ERC. Without intending to be bound by any particular theory, it is contemplated that delivery of 25-hydroxyvitamin D by modified release (extended release and/or delayed release) engages extrarenal production of 1,25-dihydroxyvitamin D without upregulating the CYP24 enzyme which catabolizes 25-hydroxyvitamin D and 1,25-dihydroxyvitamin D compounds, thus providing a more effective treatment.

Accordingly, one aspect of the disclosure herein are methods of treatment of uncomplicated acute illness due to SARS-CoV-2 in adults, e.g. adults symptomatic for no more than 7 days. Also provided are methods of treatment of uncomplicated acute illness due to SARS-CoV-2 in pediatric subjects (optionally subjects aged 12 years to 17 years of age), e.g. subjects symptomatic for no more than 7 days. Optionally, the infected subjects can be symptomatic for no more than 5 days, or no more than 3 days. In other aspects, the infected subjects can be pre-symptomatic. In still other aspects, the infected subjects can be symptomatic for no more than 10 days, or no more than 14 days.

Another aspect of the methods is treating SARS-CoV-2 infection with calcifediol, optionally ERC, including achieving a serum total 25-hydroxyvitamin D level in a patient to at least 50 ng/mL, optionally at least 60 ng/mL, and optionally greater than 60 ng/mL. Optionally, the serum total 25-hydroxyvitamin D level is maintained for all or substantially all of the duration of treatment, e.g. at least 13 days, or at least 14 days, or 19 days, or 20 days, or 21 days, or 27 days, or 28 days, or in a range of 2 to 30 days, or 2 to 28 days, or 2 to 27 days, or 2 to 21 days, or 2 to 20 days, or 2 to 19 days, or 7 to 30 days, 7 to 14 days, 7 to 20 days, or 13 to 30 days, or 14 to 30 days.

Another aspect includes treatment of subjects who have received a SARS-CoV-2 or COVID-19 vaccine (first and/or second dose, if applicable) and who have resulted in symptomatic COVID-19 disease prior to the vaccination being infective to prevent the disease.

Another aspect includes treatment of subjects who have received a SARS-CoV-2 or COVID-19 vaccine (first and/or second dose, if applicable) and who have resulted in symptomatic COVID-19 disease based on a SARS-CoV-2 variant, e.g. one having at least 80% sequence identity over the entire genome nucleotide sequence with SEQ ID NO:1.

Without being bound to a particular theory, elevation of serum total 25-hydroxyvitamin D as described herein in subjects infected with SARS-CoV-2 leads to the production of AMPs, e.g., LL37, which AMPs provide therapeutic treatment of such subjects.

Accordingly, the present disclosure provides methods of treating a SARS-CoV-2 infection. In embodiments, the method comprises administering to a subject in need thereof an effective amount of a 25-hydroxyvitamin D compound to treat the SARS-CoV-2 infection. The disclosure also provides related uses of 25-hydroxyvitamin D to treat a SARS-CoV-2 infection, and uses of 2-hydroxyvitamin D in the manufacture of a medicament to treat a SARS-CoV-2 infection.

The present disclosure also provides methods of reducing viral load of a subject in need thereof and infected with SARS-CoV-2. In exemplary embodiments, the method comprises administering to the subject an effective amount of a 25-hydroxyvitamin D compound to reduce the subject's SARS-CoV-2 viral load.

Methods of increasing an immune response in a subject infected with SARS-CoV-2 are further provided by the present disclosure. In embodiments, the method comprises administering to the subject a sufficient amount of 25-hydroxyvitamin D compound.

In aspects, the method of treating a SARS-CoV-2 infection includes treating and/or avoiding a hyperinflammatory response, e.g. Cytokine Release Syndrome, a.k.a. a cytokine storm. Accumulating data suggest that hyperactive immune response to COVID-19 poses unique risks to the cardiovascular system and play a role in disease severity. As the disease progresses, a concomitant rise in inflammatory cytokine levels may drive the depletion and exhaustion of T cell populations. On the other hand, vitamin D is an immune-modulating agent and has been implicated in the pathophysiology of autoimmune diseases, including systemic lupus erythematosus and multiple sclerosis. Vitamin D insufficiency is a risk factor for multiple sclerosis and correlates with the disease severity. Thus, it is contemplated that appropriate treatment with 25-hydroxyvitamin D may help to mitigate the overactive immune system and ameliorate immune cell exhaustion, in addition to up-regulating anti-microbial protein LL37 to combat COVID-19. Appropriate treatment with 25-hydroxyvitamin D may help to mitigate an overactive, adaptive T- and B-lymphocyte immune system. Studies have shown elevated levels of inflammation-inducing cytokines in the blood of hospitalized COVID-19 patients. Huang et al. described that plasma IL-1β, IL-1RA, IL-7, IL-8, IL-9, IL-10, basic FGF, GCSF, GMCSF, IFNγ, IP10, MCP1, MIP1A, MIP1B, PDGF, TNFα, and VEGF concentrations were higher in both ICU patients and non-ICU patients than in healthy adults. Huang et al. “Clinical features of patients infected with 2019 novel coronavirus in Wuhan, China” Lancet. 2020 15-21 February; 395(10223): 497-506. Zhang et al. described that IL-6 levels are elevated in SARS-Cov-2 infected patients. Zhang et al. “The cytokine release syndrome (CRS) of severe COVID-19 and Interleukin-6 receptor (IL-6R) antagonist Tocilizumab may be the key to reduce the mortality” Int J Antimicrob Agents. 2020 Mar. 29: 105954. In embodiments, the method comprises administering to the subject a sufficient amount of 25-hydroxyvitamin D compound to normalize one or more pro-inflammatory cytokines, or to avoid elevation of one or more pro-inflammatory cytokines, e.g. compared to untreated patients. Herold et al. reported that elevated IL-6 was strongly associated with the need for mechanical ventilation, and that the maximal IL-6 level (cutoff 80 μg/mL) for each patient during disease predicted respiratory failure. The risk of respiratory failure for patients with IL-6 levels of 80 μg/mL was 22 times higher compared to patients with lower IL-6 levels. Herold et al. “Level of IL-6 predicts respiratory failure in hospitalized symptomatic COVID-19 patients” medRxiv preprint Apr. 10, 2020 (doi: https://doi.org/10.1101/2020.04.01.20047381). Gong et al. reported correlations between IL-6, IL-8, IL-10, and TNFα levels of severity of disease, including that critical patients had higher proportions of patients with IL-6>100 μg/mL, IL-8>62 μg/mL, IL-10>20 μg/mL, and TNFα>11 μg/mL. Gong et al. “Correlation Analysis Between Disease Severity and Inflammation-related Parameters in Patients with COVID-19 Pneumonia” medRxiv preprint Feb. 27, 2020 (doi: https://doi.org/10.1101/2020.02.25.20025643). In addition Gong et al. reported that severe patients had higher proportions of patients with levels of IL-6>30 μg/mL, IL-8>31 μg/mL, IL-10>9.1 μg/mL, and TNFα>8.1 μg/mL. Accordingly, an aspect of the method herein includes administering 25-hydroxyvitamin D therapy to avoid excessive production of pro-inflammatory cytokines. An aspect of the method herein includes administering 25-hydroxyvitamin D therapy to keep IL-6 levels below 100 μg/mL, or 80 μg/mL, or 30 μg/mL. Another aspect of the method herein includes administering 25-hydroxyvitamin D therapy to keep IL-8 levels below 62 μg/mL, or 31 μg/mL. Another aspect of the method herein includes administering 25-hydroxyvitamin D therapy to keep IL-10 levels below 20 μg/mL, or 9.1 μg/mL. Another aspect of the method herein includes administering 25-hydroxyvitamin D therapy to keep TNFα levels below 11 μg/mL, or 8.1 μg/mL.

In aspects, the method of treating a SARS-CoV-2 infection includes treating and/or avoiding a pediatric inflammatory syndrome associated with SARS-CoV-2 infection, which has been referred to as Pediatric Multisystem Inflammatory Syndrome temporally associated with SARS-COV-2 (PIMS-TS) and Multisystem Inflammatory Syndrome in Children (MIS-C). It is suspected that in children with PMIS-TS, the virus causes the immune system to overreact and cause widespread inflammation throughout the body. A World Health Organization scientific brief a presentation of acute illness accompanied by a hyperinflammatory syndrome, leading to multiorgan failure and shock. “Multisystem inflammatory syndrome in children and adolescents temporally related to COVID-19”, WHO Scientific Brief, May 15, 2020 (available at https://www.who.int/news-room/commentaries/detail/multisystem-inflammatory-syndrome-in-children-and-adolescents-with-covid-19). The syndrome has been defined by (a) children and adolescents 0-19 years of age with fever >3 days; and (b) two of the following: (1) rash or bilateral non-purulent conjunctivitis or muco-cutaneous inflammation signs (oral, hands or feet), (2) hypotension or shock, (3) features of myocardial dysfunction, pericarditis, valvulitis, or coronary abnormalities (including ECHO findings or elevated Troponin/NT-proBNP), (4) evidence of coagulopathy (by PT, PTT, elevated d-Dimers), (5) acute gastrointestinal problems (diarrhoea, vomiting, or abdominal pain); and (c) elevated markers of inflammation such as ESR, C-reactive protein, or procalcitonin; and (d) no other obvious microbial cause of inflammation, including bacterial sepsis, staphylococcal or streptococcal shock syndromes; and (e) evidence of SARS-CoV-2 infection (by RT-PCR, antigen test or serology positive), or likely contact with patients with SARS-CoV-2 infection. In embodiments, the method comprises administering to the subject a sufficient amount of 25-hydroxyvitamin D compound to normalize markers of inflammation, or reduction of related symptoms, or to avoid elevation of one or markers of inflammation, or to avoid the related symptoms, e.g. compared to untreated patients.

In aspects, the method of treating a SARS-CoV-2 infection includes decreasing the duration of time from symptom onset until the return to usual health, compared to an untreated patient. For example, the duration of time from symptom onset until the return to usual health can be less than 20 days, or less than 13 days. In aspects, the method of treating a SARS-CoV-2 infection includes decreasing the duration of time from symptom onset until the return to usual activities, compared to an untreated patient. For example, the duration of time from symptom onset until the return to usual activities can be less than 10 days, or less than 7 days.

Symptoms can be assessed using the InFLUenza Patient-Reported Outcome (FLU-PRO©) questionnaire, e.g. by tracking recorded results over time. See Powers et al. Development of the Flu-PRO: a patient-reported outcome (PRO) instrument to evaluate symptoms of influenza. BMC Infect Dis 2016; 16: 1 and Powers et al. Reliability, Validity, and Responsiveness of InFLUenza Patient-Reported Outcome (FLU-PRO©) Scores in Influenza-Positive Patients, Value in Health, Vol. 21, Issue 2, 2018, p. 210-218.

The FLU-PRO© questionnaire is a 32-item daily diary that provides a measure of the presence and severity of patient-reported influenza symptoms. The FLU-PRO is a 32-item daily diary that provides a measure of the presence and severity of patient-reported symptoms across six body systems: nose (4 items: runny or dripping, congestion or stuffy, sneezing, sinus pressure), throat (3 items: scratchy or itchy throat, sore or painful throat, difficulty swallowing), eyes (3 items: teary or watery eyes, sore or painful eyes, eyes sensitive to light), chest/respiratory (7 items: trouble breathing, chest congestion, chest tightness, dry or hacking cough, wet or loose cough, coughing, coughed up mucus or phlegm), gastrointestinal (4 items: felt nauseous (feeling like you wanted to throw up), stomach ache, vomit (frequency), diarrhea (frequency)), and body/systemic (11 items: felt dizzy, head congestion, headache, lack of appetite, sleeping more than usual, body aches or pains, weak or tired, chills or shivering, felt cold, felt hot, sweating). Subjects are asked to rate each sign or symptom on a five-point ordinal scale, with higher scores indicating a more frequent sign or symptom. For 27 of the items, the scale is: 0 (not at all), 1 (a little bit), 2 (somewhat), 3 (quite a bit), and 4 (very much). For the five remaining items, severity is assessed in terms of numerical frequency of occurrence: vomiting or diarrhea (0 times, 1 time, 2 times, 3 times, or 4 or more times), with frequency of sneezing, coughing, and coughed up mucus or phlegm evaluated on a scale from 0 (never) to 4 (always). See Han et al., Using the Influenza Patient-reported Outcome (FLU-PRO) diary to evaluate symptoms of influenza viral infection in a healthy human challenge model. BMC Infect Dis 18, 353 (2018).

Blair et al., in “The Clinical Course of COVID-19 in the Outpatient Setting: A Prospective Cohort Study,” Open Forum Infect Dis. 2021 Jan. 5; 8(2):ofab007, reported that untreated COVID-19 participants returned to their usual health a median (IQR) of 20 (13-38) days from symptom onset, and to usual activities a median (IQR) of 17 (11-28) days from symptom onset. Blair et al. also reported on duration and prevalence of various symptoms.

In aspects, the method of treating a SARS-CoV-2 infection includes decreasing the mean total FLU-PRO© symptom score, compared to an untreated patient. In aspects, the method of treating a SARS-CoV-2 infection includes decreasing mean symptom scores in one or more domains (nose, throat, eyes, chest/respiratory, gastrointestinal, and body/systemic) using the FLU-PRO© questionnaire, compared to an untreated patient. In aspects, the method of treating a SARS-CoV-2 infection includes decreasing mean nose domain symptom scores using the FLU-PRO© questionnaire, compared to an untreated patient. In aspects, the method of treating a SARS-CoV-2 infection includes decreasing mean throat domain symptom scores using the FLU-PRO© questionnaire, compared to an untreated patient. In aspects, the method of treating a SARS-CoV-2 infection includes decreasing mean eyes domain symptom scores using the FLU-PRO© questionnaire, compared to an untreated patient. In aspects, the method of treating a SARS-CoV-2 infection includes decreasing mean chest/respiratory domain symptom scores using the FLU-PRO© questionnaire, compared to an untreated patient. In aspects, the method of treating a SARS-CoV-2 infection includes decreasing mean gastrointestinal domain symptom scores using the FLU-PRO© questionnaire, compared to an untreated patient. In aspects, the method of treating a SARS-CoV-2 infection includes decreasing mean body/systemic domain symptom scores using the FLU-PRO© questionnaire, compared to an untreated patient. In aspects, the method of treating a SARS-CoV-2 infection includes decreasing one or more individual symptom scores using the FLU-PRO© questionnaire, compared to an untreated patient. In aspects, the method of treating a SARS-CoV-2 infection includes decreasing prevalence and/or duration of ageusia, compared to an untreated patient. In aspects, the method of treating a SARS-CoV-2 infection includes decreasing prevalence and/or duration of anosmia, compared to an untreated patient. In aspects, the method of treating a SARS-CoV-2 infection includes decreasing prevalence and/or duration of chills, compared to an untreated patient. In aspects, the method of treating a SARS-CoV-2 infection includes decreasing prevalence and/or duration of cough, compared to an untreated patient. In aspects, the method of treating a SARS-CoV-2 infection includes decreasing prevalence and/or duration of diarrhea, compared to an untreated patient. In aspects, the method of treating a SARS-CoV-2 infection includes decreasing prevalence and/or duration of dyspnea, compared to an untreated patient. In aspects, the method of treating a SARS-CoV-2 infection includes decreasing prevalence and/or duration of fever, compared to an untreated patient. In aspects, the method of treating a SARS-CoV-2 infection includes decreasing prevalence and/or duration of headache, compared to an untreated patient. In aspects, the method of treating a SARS-CoV-2 infection includes decreasing prevalence and/or duration of myalgias, compared to an untreated patient. In aspects, the method of treating a SARS-CoV-2 infection includes decreasing prevalence and/or duration of sore throat, compared to an untreated patient. In aspects, the method of treating a SARS-CoV-2 infection includes decreasing prevalence and/or duration of weakness/fatigue, compared to an untreated patient.

For the compositions and methods described herein, optional features, including but not limited to components, compositional ranges thereof, substituents, conditions, and steps, are contemplated to be selected from the various aspects, embodiments, and examples provided herein. While the methods and compositions described herein are capable of embodiments in various forms, the description hereafter includes specific embodiments with the understanding that the disclosure is illustrative, and is not intended to limit the invention to the specific embodiments described herein.

SARS-CoV-2 is a positive-sense single-stranded RNA virus with genetic similarity to bat coronaviruses and was first isolated in January 2020 from patients in Wuhan, China (Hui et al., (2020) supra. SARS-CoV-2 is a species of the Betacoronavirus genus, which is part of the Orthocornoavirinae subfamily, which, in turn, is part of the Coronaviridae family. The complete genome sequences of different strains of SARS-CoV-2 have been determined and are publicly available online at the GenBank database of the National Center for Biotechnology Information (NCBI) website (ncbi.nlm.nih.gov). One complete genome sequence which is considered as the reference genome sequence is available as GenBank Accession No. NC_045512. This GenBank record, which also provides the amino acid sequences of the encoded proteins, are provided herein as (SEQ ID NO: 1). The SARS-CoV-2 genome sequence encodes several proteins, including, but not limited to a spike (S) protein (SEQ ID NO: 2), membrane protein (SEQ ID NO: 3), envelope protein (SEQ ID NO: 4), or nucleocapsid protein (SEQ ID NO: 5). Other SARS CoV-2 proteins include but are not limited to ORFlab polyprotein (QIQ50091.1) (SEQ ID NO: 6), ORF3a protein (QIQ50093.1) (SEQ ID NO: 7), envelope protein (QIQ50094.1) (SEQ ID NO: 8), membrane glycoprotein (QIQ50095.1) (SEQ ID NO: 9), ORF6 protein (QIQ50096.1) (SEQ ID NO: 10), ORF7a protein (QIQ50097.1) (SEQ ID NO: 11), ORF8 protein (QIQ50098.1) (SEQ ID NO: 12), nucleocapsid protein (QIQ50099.1) (SEQ ID NO: 13) and ORF10 protein (QIQ50100.1) (SEQ ID NO: 14). As used herein unless explicitly stated otherwise, the term “SARS-CoV-2” encompasses to a virus characterized by SEQ ID NO:1 and derivatives or variants thereof having at least 80% sequence identity over the entire genome nucleotide sequence. In embodiments, derivatives can have at least 85%, 88%, 90%, 91%, 92%, 93%, 94%, 95%, 96%, 97%, 98%, 99%, 99.5%, 99.8%, 99.9%, or 99.99% percent sequence identity over the entire genome nucleotide sequence.

Currently, it is believed that SARS-CoV-2 entry into host cells requires binding of the virus spike (S) protein to ACE2 expressed by the host cells. Successful entry of SARS-Cov-2 into host cells also requires proteolytic processing of the S protein by the serine protease TMPRSS. See, e.g., Hoffmann et al., Cell 181: 1-10 (2020). After host cell entry, the single-stranded RNA virus proceeds to utilize host cell machinery (e.g., host cell ribosomes) to replicate its RNA genome and its viral proteins in the host cell cytoplasm.

### Treatment of SARS-CoV-2 Infection

The present disclosure provides methods of treating a SARS-CoV-2 infection. In exemplary embodiments, the method comprises administering to a subject in need thereof an effective amount of a 25-hydroxyvitamin D compound to treat the SARS-CoV-2 infection. For purposes herein, the SARS-CoV-2 infection may be diagnosed based on positive viral nucleic acid test result on throat or nasal swab samples, irrespective of clinical signs and symptoms. In some aspects, the SARS-CoV-2 infection may be one that is clinically-diagnosed based on symptoms and exposures only. In exemplary aspects, the SARS-CoV-2 infection may be one that is clinically-diagnosed based on the presence of lung imaging features consistent with coronavirus pneumonia.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, in principle, the methods of treating a SARS-CoV-2 infection of the present disclosure can provide any amount or any level of treatment benefit over untreated patients.

Furthermore, the treatment provided by the method may include treatment of one or more symptoms or signs of the SARS-CoV-2 infection being treated. For instance, the treatment method of the present disclosure may reduce the severity or eliminate one or more symptoms of the SARS-CoV-2 infection, including, but not limited to fever, fatigue, cough, shortness of breath, difficulty breathing, chills, myalgia, headache, sore throat, loss of taste, or loss of smell. In one aspect, the symptom of SARS-CoV-2 infection include one or more of aches, chills, sore throat, nausea, and diarrhea. In another aspect, the symptom of SARS-CoV-2 infection include one or more of fever, cough, shortness of breath, difficulty breathing, chills, myalgia, headache, sore throat, and loss of taste or smell. Accordingly, the present disclosure provides methods of reducing one or more of fever, fatigue, cough, shortness of breath, difficulty breathing, aches, chills, myalgia, headache, sore throat, nausea, loss of taste, loss of smell, and diarrhea in a subject with a SARS-CoV-2 infection. In various aspects, a subject with a SARS-CoV-2 infection exhibits emergency warning signs, including, but not limited to trouble breathing, persistent pain or pressure in the chest, new confusion or inability to arouse, bluish lips. Accordingly, the present disclosure provides methods of preventing or reducing one or more emergency warning signs. In various aspects, a subject with a SARS-CoV-2 infection develops pneumonia (e.g., coronavirus pneumonia) or exhibits pneumonia-like symptoms. Accordingly, the present disclosure provides methods of preventing or treating the pneumonia or reducing the pneumonia-like symptoms of the SARS-CoV-2 infected subject.

Also, the treatment provided by the methods of the present disclosure may encompass slowing the progression of the SARS-CoV-2 infection. For example, the treatment provided by the methods of the present disclosure may reduce the SARS-CoV-2 viral load in a subject. Accordingly, the present disclosure additionally provides methods of reducing viral load of a subject in need thereof and infected with SARS-CoV-2. In exemplary embodiments, the method comprises administering to the subject an effective amount of a 25-hydroxyvitamin D compound to reduce the subject's SARS-CoV-2 viral load. Also, for example, the spectrum of SARS-CoV-2 infection reportedly ranges from mild to critical. Nonpneumonia or mild pneumonia cases were considered as “mild”, while “severe” cases exhibited dyspnea, respiratory frequency ≥30/min, blood oxygen saturation ≤93%, partial pressure of arterial oxygen to fraction of inspired oxygen ratio <300 and/or lung infiltrates >50% within 24 to 48 hours, and “critical” cases exhibited respiratory failure, septic shock, and/or multiple organ dysfunction or failure (Wu and McGoogan, JAMA doi: 10.1001/jama.2020.2648). Accordingly, the methods of the present disclosure may slow, delay, or prevent the progression of the mild SARS-CoV-2 infection to a severe SARS-CoV-2 infection or slow, delay, or prevent the progression of a severe SARS-CoV-2 infection to a critical SARS-CoV-2 infection. Also the present disclosure provides methods of treating a mild SARS-CoV-2 infection, a severe SARS-CoV-2 infection, or a critical SARS-CoV-2 infection.

The term “treat” also encompasses prophylactic treatment. For instance, a subject may be infected with SARS-CoV-2 but the subject has not yet developed COVID-19, a mild to serious respiratory disease caused by SARS-CoV-2 infection, wherein subjects exhibit fever, a deep, dry cough, shortness of breath, which can quickly become life threatening. Accordingly, the treatment provided by the presently disclosed method may delay the onset or reoccurrence/relapse of COVID-19. In aspects, the method delays the onset of COVID-19 by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of COVID-19. In aspects, the method reduces the risk of the disease 2-fold, 5-fold, 10-fold, 20-fold, 50-fold, 100-fold, or more. Accordingly, the present disclosure provides methods of delaying the onset of COVID-19 in a subject infected with SARS-CoV-2.

The degree of improvement (e.g. reduction in incidence and/or severity of symptoms or outcomes) can be in comparison to the projected measure if the patient had not been treated, e.g. based on a comparison of treated and untreated population groups, e.g. as described in the Example herein.

As used herein, the term “reduce” and words stemming therefrom may not be a 100% or complete reduction. Rather, there are varying degrees of reduction of which one of ordinary skill in the art recognizes as having a potential benefit or therapeutic effect. The presently disclosed methods may in principle reduce symptom(s), reduce the viral load, or reduce the risk of COVID-19 to any amount or level compared to untreated patients. In embodiments, the reduction provided by the methods is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

The present disclosure additionally provides methods of increasing an immune response in a subject infected with SARS-CoV-2. In exemplary embodiments, the method comprises administering to the subject a 25-hydroxyvitamin D compound. In various instances, the immune response is an adaptive immune response, without over-activating the adaptive immune system. In various aspects, administration of the 25-hydroxyvitamin D compound increases humor immunity and/or cell-mediated immunity against SARS-CoV-2. For instance, the 25-hydroxyvitamin D compound administered to the subject may lead to the production of neutralizing antibodies which bind to a SARS-CoV-2 protein and prevent their entry into host cells. Also, for example, the 25-hydroxyvitamin D compound administered to the subject may lead to activation of phagocytes, antigen-specific cytotoxic T-lymphocytes, and the release of various cytokines in response to the virus.

As used herein, the term “increase” and words stemming therefrom may not be a 100% or complete increase. Rather, there are varying degrees of an increase of which one of ordinary skill in the art recognizes as having a potential benefit or therapeutic effect. The presently disclosed methods may in principle increase an immune response to any amount or level compared to untreated patients. In embodiments, the increase provided by the methods is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase).

In aspects, the immune response is an innate immune response. In aspects, the innate immune response comprises recruitment of immune cells to sites of infection, activation of the complement cascade, or removal of antigens present in organs tissue, blood and lymph, through white blood cells (e.g., mast cells, phagocytes, including macrophages, neutrophils, and dendritic cells, basophils, eosinophils natural killer cells and the like). In various aspects, the amount is effective to increase immune cell-mediated synthesis of an antimicrobial peptide (AMP) in the subject. In some instances, the AMP is LL37, or FALL-39, or both. The immune cell which mediates synthesis of the AMP can be one or more cell types in the group of a monocyte, a macrophage, and a dendritic cell.

In embodiments, a method of the present disclosure will reduce hospitalization of treated subjects, compared to untreated subjects (e.g., incidence and/or duration of hospitalizations). In embodiments, a method of the present disclosure will reduce emergency room visits of treated subjects, compared to untreated subjects (e.g., incidence and/or duration of emergency room visits). In embodiments, a method of the present disclosure will reduce requirement for mechanical ventilation of treated subjects, compared to untreated subjects. In embodiments, a method of the present disclosure will reduce mortality rates of treated subjects, compared to untreated subjects. In embodiments, a method of the present disclosure will reduce the incidence of Serious Adverse Events of treated subjects, compared to untreated subjects, wherein a SAE is defined as 1) death; 2) hospitalization; 3) a life-threatening event (defined as a participant at immediate risk of death at the time of the event); 4) an event that results in a persistent or significant disability/incapacity; and 5) any other important medical event that may not result in one of the above outcomes that may be considered serious when, based upon appropriate medical judgment, the event may jeopardize the subject and may require medical or surgical intervention to prevent one of the outcomes listed above. In embodiments, the treatment will reduce the severity and/or duration of COVID-19 illness as evidenced by recorded severity of six symptoms, individually or average thereof, (cough, difficulty breathing, fatigue, head-ache, myalgia, and feverishness), or alternatively eight symptoms, individually or average thereof, (fever, cough, sore throat, malaise, headache, myalgia (muscle pain), gastrointestinal symptoms and shortness of breath with exertion) using a 4-point numeric rating scale (NRS; 0, absent; 3, severe) twice daily for 14 days, or 20 days, or 21 days, or 35 days, or 42 days, compared to placebo (Treanor et al., JAMA vol. 283, no. 8, Feb. 23, 2000). In embodiments, the treatment will reduce the severity and/or duration of COVID-19 illness as evidenced by recorded severity of one or more symptoms (fever, cough, shortness of breath (optionally shortness of breath with exertion), difficulty breathing, chills, malaise, myalgia, headache, gastrointestinal symptoms, sore throat, and loss of taste or smell) using a 4-point NRS (0, absent; 3, severe) twice daily for 14 days, or 20 days, or 21 days, or 27 days, or 28 days, or 35 days, or 42 days, compared to placebo (Treanor et al., JAMA vol. 283, no. 8, Feb. 23, 2000). For example, a method of the present disclosure will reduce (compared to placebo) time to resolution of illness, defined as time from study drug initiation to time of alleviation of symptoms, among individuals with SARS-COV-2 infection. Symptom alleviation can be considered to occur at the start of the first 24-hour period in which all six symptoms are scored 1 or less (mild or none) and remain so for 24 hours. In addition or in the alternative, the effect of treatment on the severity of illness can be assessed by an area under the curve analysis of total symptom scores, wherein a method according to the disclosure herein provides an improved effect compared to placebo. In addition, on each day during a dosing period as described herein, subjects under treatment can record their ability to perform usual activities on a diary card using an 11-point NRS (unable to perform normal activity, 0; fully able to perform normal activity, 10), wherein return to normal activity is defined as the time (in hours) from treatment initiation to the first 24-hour period in which the subject returns to a normal level of activity, wherein a method according to the disclosure herein provides a reduced duration compared to placebo. In addition, subjects under treatment can complete a visual analog scale of their opinion of overall health status including normal, pre-SARS-CoV-2 health on an 11-point scale (0, worst health and 10, best possible health). Following this, they can record their assessment of health status at baseline and over a 24-hour period once daily in the evening, wherein return to normal health is defined as the time (in hours) from treatment initiation to the first 24-hour period in which the subject returns to a normal level of health, wherein a method according to the disclosure herein provides a reduced duration compared to placebo. The use of these scales has been validated in a pilot study conducted among English-speaking volunteers during the influenza season in Australia in 1997 (Treanor et al 2000). Oral temperature can also be taken by the patient with a digital thermometer twice daily and recorded on the diary card, wherein a method according to the disclosure herein provides a reduced duration of return to normal temperature, compared to placebo. As used herein, a comparison to placebo can be an individual measure which is based on population basis.

In embodiments, SARS-CoV-2 subjects receiving 25-hydroxyvitamin D can, on each day during the dosing period: i) record twice daily the severity of six SARS CoV-2 symptoms (cough, difficulty breathing, fatigue, head-ache, myalgia, and feverishness) using a 4-point NRS (0, absent; 3, severe); ii) record once daily their ability to perform usual activities on a diary card using an 11-point NRS (unable to perform normal activity, 0; fully able to perform normal activity, 10); and, iii) complete once daily a record of their opinion of overall health status using an 11-point NRS (0, worst health and 10, best possible health). These measures (individually and in combinations) can be used for comparison with untreated subjects, to gauge the treatment effect.

In embodiments, SARS-CoV-2 subjects receiving 25-hydroxyvitamin D can, on each day during the dosing period: i) record twice daily the severity of eight SARS CoV-2 symptoms (
fever, cough, sore throat, malaise, headache, muscle pain, gastrointestinal symptoms and shortness of breath with exertion) using a 4-point NRS (0, absent; 3, severe); and further optionally ii) record once daily their ability to perform usual activities using an 11-point NRS (unable to perform normal activity, 0; fully able to perform normal activity, 10); and, iii) complete once daily a record of their opinion of overall health status using an 11-point NRS (0, worst health and 10, best possible health). These measures (individually and in combinations) can be used for comparison with untreated subjects, to gauge the treatment effect.

In aspects of the presently disclosed methods, characteristics of treated subject's differ from those of subjects that did not receive 25-hydroxyvitamin D, and subjects that active lower serum levels of 25-hydroxyvitamin D. In aspects, the administration of 25-hydroxyvitamin D accelerates the acute, subacute, and late inflammation-response-related methylome and transcriptome in peripheral blood mononuclear cells compared to placebo. In various aspects, the administration of 25-hydroxyvitamin D leads to an increase in the subject's peripheral blood mononuclear cell (PBMC) count, compared to placebo. In instances, the administration of 25-hydroxyvitamin D leads to an increase in the subject's peripheral blood neutrophil count, compared to placebo. Additionally, in instances, administration of 25-hydroxyvitamin D leads to a reduction in the subject's SARS-CoV-2 titer level or vial load, compared to placebo. Optionally, the administration of 25-hydroxyvitamin D leads to a reduction in the subject's SARS-CoV-2 titer level or viral load at Day 7 of treatment, compared to placebo. Also, for instance, the administration of 25-hydroxyvitamin D may lead to a reduction in the subject's SARS-CoV-2 titer levels or viral load at Day 14 of treatment, compared to placebo. Also, for instance, the administration of 25-hydroxyvitamin D may lead to a reduction in the subject's SARS-CoV-2 titer levels or viral load at Day 20 of treatment, compared to placebo. In aspects, administration of 25-hydroxyvitamin D leads to an increase in the subject's anti-SARS-CoV-2 antibody level (e.g. serum level), compared to placebo. In various instances, administration of 25-hydroxyvitamin D leads to an increase in the subject's anti-SARS-CoV-2 antibody level at Day 7 of treatment, compared to placebo. In various instances, administration of 25-hydroxyvitamin D leads to an increase in the subject's anti-SARS-CoV-2 antibody level at Day 14 of treatment, compared to placebo. In various instances, administration of 25-hydroxyvitamin D leads to an increase in the subject's anti-SARS-CoV-2 antibody level at Day 20 of treatment, compared to placebo. In various instances, administration of 25-hydroxyvitamin D leads to an increase in the subject's anti-SARS-CoV-2 antibody level at Day 27 or 28 of treatment, compared to placebo. In exemplary aspects, administration of 25-hydroxyvitamin D leads to an increase in the subject's serum level of LL37, and anti-SARS-CoV2 antibody level, compared to untreated subjects. In exemplary aspects, administration of 25-hydroxyvitamin D leads to a decrease in the subject's serum level of one or more of eotaxin, monocyte chemotactic protein (MCP-1), IL-12, IL-6, IL-1β, and caspase-3, compared to untreated subjects.

In aspects, administration of 25-hydroxyvitamin D leads to a reduction in the subject's clinical disease severity score compared to placebo. Optionally, with regard to the above increase(s), said increase is at Day 28 or 27 of treatment compared to pretreatment level, or at Day 28 or 27 of treatment compared to Day 7 of treatment level, or at Day 28 or 27 of treatment compared to Day 14 of treatment level. Optionally, with regard to the above increase(s), said increase is at Day 7 of treatment compared to pretreatment level, at Day 14 of treatment compared to pretreatment level, at Day 20 of treatment compared to pretreatment level, or at Day 20 of treatment compared to Day 7 of treatment level, or at Day 20 of treatment compared to Day 14 of treatment level, or at Day 14 of treatment compared to Day 7 of treatment level. Optionally, with regard to the above reduction(s), said reduction or decrease is at Day 7 of treatment compared to pretreatment level, at Day 14 of treatment compared to pretreatment level, at Day 20 of treatment compared to pretreatment level, at Day 27 of treatment compared to pretreatment level, at Day 28 of treatment compared to pretreatment level, at Day 27 or 28 of treatment compared to day 7 of treatment level, at Day 20 of treatment compared to Day 7 of treatment level, at Day 20 of treatment compared to Day 14 of treatment level, or at Day 14 of treatment compared to Day 7 of treatment level.

For example, a method disclosed herein can improve outcomes (e.g. compared between Day 1 and Day 42) based on the change of symptom score for COVID-19, depending on serum 25D concentrations achieved at Days 7, 14, 21 and 28 (25D<50 ng/mL or ≥50 ng/mL). A method disclosed herein can improve outcomes (e.g. compared between Day 1 and Day 42) based on the change of symptom score for COVID-19, in patients with severe hypovitaminosis D (serum 25D<20 ng/mL) at Day 0, depending on serum 25D concentrations achieved at Days 7, 14, 21 and 28 (25D<50 ng/mL or ≥50 ng/mL).

With regard to the presently disclosed methods, the amount of the 25-hydroxyvitamin D compound is effective to achieve and maintain a serum total 25-hydroxyvitamin D level of at least 50 ng/mL in the subject during the treatment period. Optionally, the amount is effective to achieve and maintain a serum total 25-hydroxyvitamin D level of at least 60 ng/mL during the treatment period. The method can include achieving such serum levels, e.g. at least or greater than 50 ng/mL, or at least or greater than 60 ng/mL, in the first 24 hours of treatment. The serum level during treatment can be 200 ng/mL or less, or 100 ng/mL or less, in embodiments. For example, the method can include achieving a serum level of at least 50 ng/mL and less than 100 ng/mL in the first 24 hours of treatment. In various instances, the amount is effective to achieve and maintain a serum total 25-hydroxyvitamin D level greater than 60 ng/mL in the subject, e.g., greater than 70 ng/mL, greater than 80 ng/mL, greater than 90 ng/mL, greater than 100 ng/mL, greater than 125 ng/mL, greater than 150 ng/mL, greater than 175 ng/mL, greater than 200 ng/mL, greater than 250 ng/mL, greater than 300 ng/mL, greater than 350 ng/mL, greater than 400 ng/mL, greater than 450 ng/mL, or up to 500 ng/mL during the treatment period, or in a range of about 50 ng/mL to about 100 ng/mL, or about 60 ng/mL to about 100 ng/mL, or greater than 60 ng/mL to about 100 ng/mL during the treatment period.

In various aspects, the 25-hydroxyvitamin D compound is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly.

In instances, the method includes a loading dose of the 25-hydroxyvitamin D compound administered to the subject before one or more maintenance doses of the 25-hydroxyvitamin D compound. In various aspects, the loading dose is greater than about 90 μg, or at least 100 μg, or at least 200 μg, or at least 250 μg, or greater than about 250 μg or greater than about 500 μg. Optionally, the loading dose is about 1200 μg or less, 1000 μg or less. In various aspects, the loading dose is about 90 μg to about 250 μg, or about 500 μg to about 900 μg, about 500 μg to about 800 μg, about 500 μg to about 700 μg, about 500 μg to about 600 μg, about 600 μg to about 1000 μg, about 700 μg to about 1000 μg, about 800 μg to about 1000 μg, or about 900 μg to about 1000 μg. In various instances, the loading dose is at least or about 900 μg±90 μg of the 25-hydroxyvitamin D compound. Any of the foregoing doses can be administered in the fasted state, e.g. at least 3 hours following a meal, including at bedtime, and without food. Any of the foregoing doses can be administered as a Rayaldee®-type formulation, as described herein, or as an extended-release oral formulation having a bioavailability of about 25%. In another aspect, the loading dose is an immediate release 25-hydroxyvitamin D formulation. For example, an immediate release oral formulation having a bioavailability 3 times that of the Rayaldee®-type formulation, or about 70%-80%, can be provided in a loading dose of about 200 μg to about 750 μg, or about 200 μg to 400 μg, or 500 μg to 600 μg, or 500 μg to 550 μg, or 532 μg, or 600 μg to 700 μg, or 650 μg to 750 μg. An immediate release loading dose can be followed by maintenance dosing by immediate release or extended release formulations, and in one type of embodiment will be followed by maintenance dosing with an extended release formulation. In embodiments, the loading dose can be the first dose, e.g. the Day 1 dose. In other embodiments, the loading dose is administered in divided doses, e.g. over a period of one or more days, for example 1 to 5 days, or 2 to 5 days. For example, the loading dose can be administered over a period of two or more days, or three days, e.g. a 900 μg loading dose can be administered as 300 μg per day for Days 1, 2, and 3, followed by maintenance doses as described herein, or a 900 μg loaded dose can be administered as 450 μg per day for Days 1 and 2, followed by maintenance doses as described herein. In embodiments, the loading dose is administered in the fasting state.

In various aspects, the one or more daily maintenance doses is at least 25 μg, or at least 30 μg, or greater than 30 μg, or greater than about 50 μg of the 25-hydroxyvitamin D compound. Optionally, each maintenance dose is less than or about 100 μg of the 25-hydroxyvitamin D compound. In various instances, each maintenance dose is about 50 μg to about 100 μg, about 50 μg to about 80 μg, about 50 μg to about 70 μg, about 50 μg to about 60 μg, about 60 μg to about 100 μg, about 70 μg to about 100 μg, about 80 μg to about 100 μg, or about 90 μg to about 100 μg. In various instances, each maintenance dose is about 60 μg±6 μg of the 25-hydroxyvitamin D compound. Any of the foregoing doses can be administered in the fasted state, e.g. at least 3 hours following a meal, including at bedtime, and without food. Any of the foregoing doses can be administered as a Rayaldee®-type formulation, as described herein, or as an extended-release oral formulation having a bioavailability of about 25%. In the alternative, the maintenance doses can be administered in an immediate release formulation, e.g. one having a bioavailability of about 70-80%. In embodiments, the maintenance doses are administered in the fasting state. Maintenance doses can be administered daily, or a daily maintenance dose can be administered in divided doses throughout the day, or an equivalent amount of 25-hydroxyvitamin D can be administered on a frequency less than daily, e.g. 60 μg every other day in place of 30 μg daily, or about 210 μg weekly in place of 30 μg daily.

Loading doses and maintenance doses can further be adjusted based on a subject's body weight, i.e. such that patients having relatively high BMI levels receive relatively more 25-hydroxyvitamin D.

Loading doses and maintenance doses can further be adjusted based on a subject's serum total 25-hydroxyvitamin D level. For example, a patient who is not vitamin D insufficient or deficient but still has a serum total 25-hydroxyvitamin D level below 50 ng/ml or 60 ng/ml can receive a relatively lower amount of loading dose than a subject who is vitamin D insufficient or deficient.

It is contemplated that doses, e.g. loading doses and/or maintenance doses, can be provided in an amount to maintain a subject's serum total 25-hydroxyvitamin D level of at least 40 ng/ml, or at least 50 ng/ml, or at least 60 ng/ml, for example, in a range of 40 ng/ml to 100 ng/ml, or 50 ng/ml to 200 ng/ml, 50 ng/ml to 100 ng/ml, or 60 ng/ml to 100 ng/ml, or 40 ng/ml to 80 ng/ml.

In various instances, the method comprises administering a daily maintenance dose to the subject, optionally, for at least 3 days, 5, days, 1 week, 10 days, 12 days, 13 days, 2 weeks, 19 days, 20 days, 3 weeks, 26 days, 4 weeks, or more. Optionally, the method comprises administering to the subject a loading dose of 900 μg of the 25-hydroxyvitamin D compound followed by daily maintenance doses for at least 1 week, or at least 2 weeks, or at least 19 days, at least 20 days, or at least 26 days. In various instances, each daily maintenance dose can be 60 μg of the 25-hydroxyvitamin D compound. Optionally, such method comprises administering daily maintenance doses for at least 13 days, or at least 2 weeks, or at least 19 days, or at least 20 days, optionally at least 3 weeks, or at least 26 days, or at least 4 weeks, or more. For example, in a method of preventing SARS-Cov-2 infection, or the progression of infection (e.g. from incubation to prodrome stage, or prodrome stage to COVID-19 disease stage), the method can include maintenance dosing (e.g. 60 μg/day of a Rayaldee®-type formulation) without an initial loading dose, or with a relatively low loading dose.

In the fasting state, a loading dose of 900 μg of a Rayaldee®-type formulation (approximately 25% bioavailability) will raise serum total 25-hydroxyvitamin D level within about 10 hours by about 20 ng/mL to 30 ng/mL, depending on the subject's body weight (the higher the body weight, the lower the expected increase in serum total 25-hydroxyvitamin D). Each daily 60 μg maintenance dose of a Rayaldee®-type formulation will increase serum total 25-hydroxyvitamin D by another 0.6 ng/mL. It follows that subjects having a baseline serum total 25-hydroxyvitamin D level of about 25 ng/mL will reach about 45 ng/mL to 55 ng/mL level after the loading dose, about 53-63 ng/mL after 14 days of maintenance dosing, and 61-71 ng/mL after 26 days of maintenance dosing, when administered in the fasting state. In other embodiments, the method and formulation can be selected to provide a serum total 25-hydroxyvitamin D level of at least 50 ng/mL, or at least 60 ng/mL, and up to 200 ng/mL, or up to 100 ng/mL, in the first 24 hours after the initial dose.

In the fed state, serum total 25-hydroxyvitamin D level will increase about 3 to 4 times more after dosing with ERC (e.g. Rayaldee®-type formulation) compared to in the fasting state. For this reason, and to improve consistency in absorption from dosing, it is contemplated that all dosing can occur at bedtime (in the fasting state, defined as at least about 3 hours after the subject's last meal, optionally at least about 4 hours after the last meal).

Immediate-release calcifediol (formulated in MCT oil), has a bioavailability that is approximately 3 times higher than that of the Rayaldee®-type formulation. Consequently, the loading and maintenance doses described above in the fasting state can be scaled (decreased) by two-thirds. Doses for other formulations, both oral and via other dosage routes, can be scaled by the person of ordinary skill based on their bioavailability and/or pharmacokinetics. For example, as the Rayaldee®-type formulation has approximately 25% bioavailability, a loading dose for another type of formulation having three times the bioavailability can be greater than about 63 μg bioavailable amount of 25-hydroxyvitamin D delivered by the formulation, or greater than about 125 μg bioavailable amount. Optionally, the loading dose is less than about 250 μg bioavailable amount of 25-hydroxyvitamin D delivered by the formulation. In various aspects, the loading dose is about 125 μg to about 300 μg, about 125 μg to about 225 μg, about 125 μg to about 200 μg, about 125 μg to about 175 μg, about 125 μg to about 150 μg, about 150 μg to about 250 μg, about 175 μg to about 250 μg, about 200 μg to about 250 μg, or about 225 μg to about 250 μg bioavailable amount. Similarly the one or more maintenance doses can be at least about 7 μg, or greater than 7 μg, or greater than about 12 μg of bioavailable 25-hydroxyvitamin D. Optionally, each maintenance dose is less than or about 25 μg of bioavailable 25-hydroxyvitamin D. In various instances, each maintenance dose can be about 12 μg to about 25 μg, about 12 μg to about 20 μg, about 12 μg to about 17 μg, about 12 μg to about 15 μg, about 15 μg to about 25 μg, about 17 μg to about 25 μg, about 20 μg to about 25 μg, or about 22 μg to about 25 μg of bioavailable 25-hydroxyvitamin D. In various instances, each maintenance dose is about 15 μg±1.5 μg of bioavailable 25-hydroxyvitamin D in such a formulation.

From another perspective, since the Rayaldee®-type formulation has a bioavailability of about 25%, dosing amounts can be expressed based on the bioavailable amount of 25-hydroxyvitamin D in any type of formulation. In various aspects, the loading dose is greater than about 22 μg, or at least 25 μg, or at least 50 μg, or at least 62 μg, or greater than about 62 μg or greater than about 125 μg of bioavailable 25-hydroxyvitamin D. Optionally, the loading dose is less than about 250 μg. In various aspects, the loading dose is about 22 μg to about 62 μg, or about 125 μg to about 225 μg, about 125 μg to about 200 μg, about 125 μg to about 175 μg, about 125 μg to about 150 μg, about 150 μg to about 250 μg, about 175 μg to about 250 μg, about 200 μg to about 250 μg, or about 225 μg to about 250 μg. In various instances, the loading dose is at least or about 225 μg±22 μg of bioavailable 25-hydroxyvitamin D. Any of the foregoing doses can be administered in the fasted state, e.g. at least 3 hours following a meal, including at bedtime, and without food. In various aspects, the one or more daily maintenance doses is at least 6 μg, or at least 7 μg, or greater than 7 μg, or greater than about 12 μg of bioavailable 25-hydroxyvitamin D. Optionally, each maintenance dose is less than or about 25 μg of bioavailable 25-hydroxyvitamin D. In various instances, each maintenance dose is about 12 μg to about 25 μg, about 12 μg to about 20 μg, about 12 μg to about 18 μg, about 12 μg to about 15 μg, about 15 μg to about 25 μg, about 17 μg to about 25 μg, about 20 μg to about 25 μg, or about 22 μg to about 25 μg of bioavailable 25-hydroxyvitamin D. In various instances, each maintenance dose is about 15 μg±1.5 μg of the 25-hydroxyvitamin D compound. Any of the foregoing doses can be administered in the fasted state, e.g. at least 3 hours following a meal, including at bedtime, and without food.

Rapidly rising or excessive intracellular levels of vitamin D hormones stimulate the expression of a cytochrome P450 enzyme known as CYP24A1 in cells which contain the vitamin D receptor. The CYP24A1 enzyme catabolizes 1,25-dihydroxyvitamin D, 25-hydroxyvitamin D and vitamin D with high specificity, thereby restoring normal intracellular vitamin D hormone levels. This is an important feedback mechanism that limits excessive and potentially harmful local exposure to vitamin D hormones. Accordingly, it is contemplated to administer 25-hydroxyvitamin D in the absence of upregulating expression of CYP24A1. On the other hand, to provide a rapid immune response based on availability of 25-hydroxyvitamin D, e.g. correction of hypovitaminosis D, it is contemplated to safely raise serum total 25-hydroxyvitamin D levels within the first 24 hours of dosing, e.g. to at least 50 ng/mL, or greater than 50 ng/mL, or at least 60 ng/mL, or greater than 60 ng/mL, and optionally up to 200 ng/mL, or up to 100 ng/mL. Similarly, it is contemplated that a formulation for use in the method herein can provide an in vivo Tmax in a range of 4 to 24 hours, or 4 to 18 hours, or 4 to 16 hours, or 4 to 12 hours, or 4 to 8 hours, for example.

A patient's vitamin D metabolite ratio (VMR, calculated as 100 times the ratio of serum 24,25-dihydroxoxyvitamin D₃ to serum 25-hydroxyvitamin D₃, or the ratio of 24,25-dihydroxoxyvitamin D₃ to serum 25-hydroxyvitamin D₃ following administration of a vitamin D₃-type product, e.g. 25-hydroxyvitamin D₃) can be used as an indicator of induction of CYP24A1. See Strugnell S A, Sprague S M, Ashfaq A et al. “Rationale for Raising Current Clinical Practice Guideline Target for Serum 25-Hydroxyvitamin D in Chronic Kidney Disease” Am. J. Nephrol. 2019; 49(4):284-293. Strugnell et al. showed that in Stage 3 and 4 CKD patients having vitamin D insufficiency and SHPT, and treated with 30 or 60 μg ERC over 26 weeks, mean posttreatment VMR rose only moderately (maximum 4.8), suggesting that there was no substantial induction of CYP24A1. Similarly, as described in Example 7 below, in Stage 3 and 4 CKD patients having vitamin D insufficiency and SHPT, and treated with 60 μg ERC for 8 weeks, mean posttreatment VMR also remained below 5 (maximum about 4.2). VMR following dosing with 25-hydroxyvitamin D is dose-dependent. When a sufficiently high dose of 25-hydroxyvitamin D is administered, particularly with immediate-release 25-hydroxyvitamin D, VMR can achieve higher levels. Likewise, with sufficiently frequent repeated dosing with 25-hydroxyvitamin D, VMR can increase over time, and achieve higher levels than desired. In addition, with a sufficiently rapid and potent delivery of 25-hydroxyvitamin D, the rate of VMR increases proportionally. Accordingly, in one aspect, the methods of treatment herein optionally will employ a dosing regimen in which VMR remains substantially constant over a period of at least 28 days, further optionally during maintenance dosing period. In another aspect, the methods of treatment herein optionally will employ a dosing regimen in which VMR decreases over a period of at least 28 days, further optionally during maintenance dosing period. In another aspect, the methods of treatment herein optionally will employ an extended release dosing regimen in which the rate of change of VMR, e.g. in a period of 28 days, is less than the rate of change of VMR for a bioequivalent amount of 25-hydroxyvitamin D administered by immediate release. In another aspect, the methods of treatment herein optionally will employ a dosing regimen in which VMR does not exceed 12, or does not exceed 11, or does not exceed 5, or does not exceed 4.8. In other aspect, recognizing that the patients can benefit from correcting vitamin D insufficiency and achieving a serum total 25-hydroxyvitamin D level of at least 50 ng/ml as described herein, the methods of treatment herein optionally will employ a dosing regimen in which VMR can exceed 4.8, or 5, or 11, or 12, during a loading dose phase, and does not exceed 11, or does not exceed 5, or does not exceed 4.8 during a maintenance dosing phase. In still another aspect, the methods of treatment herein optionally will employ a dosing regimen in which VMR does not exceed 12 during a loading dose phase (e.g. is in a range of 4 to 12), and does not exceed 11 during a maintenance dosing phase (e.g. is in a range of 3 to 11).

In various aspects, the 25-hydroxyvitamin D compound is administered in a modified release formulation. As used herein, the terms “controlled release,” and “modified release” are used interchangeably and refer to the release of the administered vitamin D compound in a way that deviates from immediate release. The modified release formulation can be an extended release formulation. Optionally, the modified release formulation can include a delayed release aspect. As used herein, the terms “sustained release,” “extended release,” and “prolonged release” are used interchangeably and refer to the release of the administered vitamin D compound over a longer period of time than a comparable immediate release formulation.

The 25-hydroxyvitamin D compound may be administered to the subject by any suitable means. Formulations suitable for oral administration can consist of or include (a) liquid solutions or suspensions, such as an effective amount of the 25-hydroxyvitamin D compound dissolved or suspended in diluents, such as water, saline, orange juice, milk, oils, or other carriers; (b) capsules, sachets, tablets, lozenges, and troches, each containing a predetermined amount of the 25-hydroxyvitamin D compound, as solids or granules; (c) powders; and (d) suitable emulsions. Liquid formulations may include diluents, such as water or alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, carriers, such as oils, waxes, or other lipids, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the 25-hydroxyvitamin D compound of the present disclosure in a flavor, usually sucrose and acacia or tragacanth, as well as pastilles comprising the analog of the present disclosure in an inert base, such as gelatin and glycerin, or sucrose and acacia, emulsions, gels, and the like containing, in addition to, such other excipients as are known in the art. The 25-hyroxyvitamin D compound can be dissolved in an alcohol, e.g. ethanol, for distribution in a carrier or excipient.

The 25-hydroxyvitamin D compound can be dispersed in a polymer composition. The 25-hydroxyvitamin D compound can be embedded in a polymer network. The polymer can be water-insoluble, and optionally swellable, for example. The formulation can be a spheronized pellet formulation comprising a 25-hydroxyvitamin D compound and a pharmaceutically acceptable excipient. Such pellets optionally can be enteric coated; in the alternative, the pellets can be disposed in a capsule shell (e.g. gelatin, vegetable based, or polymer based) which is enteric coated. The formulation can include a 25-hydroxyvitamin D compound dispersed in a fatty acid glyceride mixture. The formulation can consist of or include a nano/microparticle formulation comprising a 25-hydroxyvitamin D compound and a pharmaceutically acceptable excipient. The formulation can consist of or include a lipid microparticle formulation comprising a 25-hydroxyvitamin D compound and a pharmaceutically acceptable lipid. The formulation can consist of or include a non-pareil seed formulation comprising a 25-hydroxyvitamin D compound and a pharmaceutically acceptable excipient. The formulation can consist of or include a 25-hydroxyvitamin D compound and a pharmaceutically acceptable excipient selected from one or more excipients in the group of an absorption enhancer, a spheronizing aid, a water insoluble polymer, and a binder. The formulation can consist of or include a spray-congealed lipid vitamin D formulation comprising a 25-hydroxyvitamin D compound, an extended release agent, and a surfactant. In embodiments, the formulation can be an extended release formulation, e.g. for oral use.

The 25-hydroxyvitamin D compound of the disclosure, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the 25-hydroxyvitamin D compound is formulated into a powder blend or into microparticles or nanoparticles. Suitable pulmonary formulation types are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as topical (including transdermal patch), subcutaneous, intramuscular, intraspinal, or intravenous. The 25-hydroxyvitamin D compound of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. A topical formulation can take any suitable form, e.g. cream, ointment, paste, lotion, gel, or patch.

Oils, which can be used in enteral and parenteral formulations, include petroleum, animal, vegetable, or synthetic oils. Specific examples of oils include peanut, soybean, sesame, cottonseed, corn, olive, petrolatum, and mineral. Non-digestible oils are contemplated for some embodiments. Suitable fatty acids for use in parenteral formulations include oleic acid, stearic acid, and isostearic acid. Ethyl oleate and isopropyl myristate are examples of suitable fatty acid esters.

The parenteral formulations can contain from about 0.5% to about 25% by weight of the 25-hydroxyvitamin D compound of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

In various aspects, the 25-hydroxyvitamin D compound is administered orally. In various instances, the 25-hydroxyvitamin D compound comprises 25-hydroxyvitamin D₂ or 25-hydroxyvitamin D₃, or a combination of 25-hydroxyvitamin D₂ and 25-hydroxyvitamin D₃. It is specifically contemplated that in any and every aspect and embodiment of the compositions and methods disclosed herein, the 25-hydroxyvitamin D compound can be 25-hydroxyvitamin D₃. As used herein, the term “25-hydroxyvitamin D compound” refers to one or more of 25-hydroxyvitamin D₃, 25-hydroxyvitamin D₂, 25-hydroxyvitamin D₄, 25-hydroxyvitamin D₅, or 25-hydroxyvitamin D₇, and it is contemplated that in any reference thereto a preferred embodiment is one or more of 25-hydroxyvitamin D₃ and 25-hydroxyvitamin D₂, preferably 25-hydroxyvitamin D₃. Thus, in any and all formulations described herein, it is specifically contemplated that the active can include one or both of 25-hydroxyvitamin D₂ and 25-hydroxyvitamin D₃, particularly 25-hydroxyvitamin D₃.

As used herein, the formulation comprising the 25-hydroxyvitamin D compound can be a stabilized formulation, wherein “stabilized formulation” refers to a formulation exhibiting a stable in vitro dissolution profile (according to any of the parameters described further herein) and controlled release (e.g., extended release) of a vitamin D compound in vivo, for a time following initial manufacture, e.g. following actual shelf storage or accelerated stability storage conditions. The release of the active ingredient can be measured using a suitable in vitro dissolution method, such as one of the methods already known in the art. In principle, any of the dissolution studies described in the United States Pharmacopeia, USP 43-NF 38 2S, Dissolution <711> physical tests and determinations, United States Pharmacopeial Convention, Inc., Rockville, Md., 2020; European Pharmacopoeia 2.9.3 Dissolution Test for Solid Dosage Forms, or the Japanese Pharmacopoeia 6.10 Dissolution Test, can be used to determine if a formulation is stable. For purposes of the present disclosure, the single medium in vitro dissolution method is United States Pharmacopeia, USP 43-NF 38 2S, Dissolution <711> physical tests and determinations, United States Pharmacopeial Convention, Inc., Rockville, Md., 2020, using Apparatus 2 (paddle method), as described in the embodiments below. In an alternative, dissolution characteristics can be measured using a 2-phase method, such as Method 2 in USP 43-NF 38 2S, Dissolution <711>, using Apparatus 1 or 2, optionally Apparatus 2.

A stabilized formulation according to the disclosure herein, following storage for a period of time, releases an amount of 25-hydroxyvitamin D in in vitro dissolution that does not substantially differ from the dissolution of the same formulation just after manufacturing and prior to storage. For example, in one embodiment, a formulation releases an amount of 25-hydroxyvitamin D during in vitro dissolution after exposure to storage conditions of two months at 25° C. and 60% relative humidity that varies at any given dissolution time point after four hours by 30% or less compared to the amount released at the same dissolution time point during in vitro dissolution conducted prior to exposing the formulation to the storage conditions (i.e., freshly-produced product).

The table below provides examples of advantageous degrees of storage stability contemplated for embodiments of the invention following storage at 25° C. and 60% RH, and alternatively at 40° C. and 75% RH for various times following initial manufacturing, and at various times in during dissolution testing. The degrees of storage stability are expressed in terms of the maximum deviation from nominal active potency, i.e. maximum % change from LC. Alternative embodiments of maximum deviation are also provided.

| | | | | | | | |
|---|---|---|---|---|---|---|---|
| Time | | | | | | | |
| (h) | 1 month | 3 mos. | 6 mos. | 9 mos. | 12 mos. | 18 mos. | 24 mos. |

storage at 25° C. and 60% RH

| | | | | | | | |
|---|---|---|---|---|---|---|---|
| 2 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 4 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 6 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 8 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 12 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |

storage at 40° C. and 75% RH

| | | | | | | | |
|---|---|---|---|---|---|---|---|
| 2 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 4 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 6 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 8 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 12 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |

In one type of embodiment, the formulation will have advantageous degrees of stability described in the table immediately above at multiple time points throughout the dissolution testing, e.g. at least at both 2 and 4 hour time points, optionally also at the 6 hour time point, further optionally also at the 8 hour time point, and further optionally also at the 12 hour time point, such that the dissolution profile after storage follows the dissolution profile of fresh product. Alternatively, the formulation will have advantageous degrees of stability described in the table immediately above at least at the 2, 6, and 12 hour time points. Alternatively, the formulation will have advantageous degrees of stability described in the table immediately above at least at the 4, 8, and 12 hour time points. Alternatively, the formulation will have advantageous degrees of stability described in the table immediately above at least at the 2, 4, and 6, hour time points. Alternatively, the formulation will have advantageous degrees of stability described in the table immediately above at least at the 4, 6, 8, and 12 hour time points, or at all times of 4 hours and thereafter.

In any and all of the embodiments described in the table immediately above, it is contemplated that the deviation can be positive (more release) or negative (less release) with respect to the fresh product. In one type of embodiment, it is contemplated that the deviation will be in the negative (less release) direction at multiple time points. Still further, in one type of embodiment it is contemplated that the deviation in dissolution release would have been negative (less release) at multiple time points but for the presence of the stabilizing agent in the formulation.

In any of the embodiments contemplated herein, the dissolution release profile of the formulation can have the characteristics of any one of the examples provided herein below. For example, the formulation can be characterized by a dissolution release profile providing a release of vitamin D compound of less than 30% at 2 hours, greater than 45% at 6 hours, and greater than 80% at 12 hours, and further optionally less than 60% at 6 hours.

In another type of embodiment, the formulation can be characterized by an in vitro dissolution profile providing release of vitamin D compound of less than 30% at 100 to 140 minutes, greater than 45% at 5 to 7 hours, and greater than 80% at 11 to 13 hours. In another type of embodiment, the formulation can be characterized by an in vitro dissolution profile providing release of vitamin D compound of less than 30% at 2 hours, greater than 45% at 6 hours, and greater than 80% at 12 hours. In these types of embodiments, optionally the release of vitamin D compound at 5 to 7 hours is less than 60%, or at 6 hours is less than 60%.

In another type of embodiment, the formulation can be characterized by an in vitro dissolution profile providing release of vitamin D compound of about 20% to about 40% at 2 hours, at least 35% at 6 hours, and at least 70% at 12 hours. In another type of embodiment, the formulation can be characterized by an in vitro dissolution profile providing release of vitamin D compound of about 25% to about 35% at 2 hours, at least 40% at 6 hours, and at least 75% at 12 hours. In these types of embodiments, optionally the release of vitamin D compound is 75% or less at 6 hours, or 65% or less at 6 hours, or 60% or less at 6 hours, for example.

In various instances, the formulation comprising the 25-hydroxyvitamin D compound comprises a matrix component that releasably binds the vitamin D compound and controllably releases the vitamin D compound (e.g., a lipophilic matrix), and a stabilizer (e.g. a cellulosic compound). In various instances, the stabilizing agent is a cellulosic compound. As used herein, the term “cellulosic compound” can include cellulose (C₆H₁₀O₅)ₙ or a derivative of cellulose, unless specified otherwise. In various aspects, the cellulosic compound is a cellulose ether. A “cellulose ether” is a cellulose derivative that has been chemically modified to result in partial or complete etherification of the hydroxyl groups in the cellulose molecule. Examples of cellulose derivatives which can be used as stabilizing agents include, but are not limited to, celluloronic acid, carboxy methyl cellulose, ethyl cellulose, hydroxyethyl cellulose, hydroxyl propyl cellulose, hydroxyl propyl methylcellulose, methyl cellulose, polyanionic cellulose, and combinations thereof, for example. Different grades of each cellulosic compound or stabilizing agent, corresponding to variations in, e.g., molecular weight, viscosity, solubility, and hydration, are also encompassed by the terms.

In one embodiment, a stabilized formulation comprises one or both of 25-hydroxyvitamin D₂ and 25-hydroxyvitamin D₃, a wax matrix, and a cellulosic compound. In one aspect, a stabilized formulation comprises one or both of 25-hydroxyvitamin D₂ and 25-hydroxyvitamin D₃, a wax matrix, and a cellulosic stabilizing agent. In another aspect, the formulation comprises one or both of 25-hydroxyvitamin D₂ and 25-hydroxyvitamin D₃, a wax matrix, and an effective amount of a cellulosic compound to provide an advantageous degree of stability as described herein, e.g. with respect to the table immediately above or consistent with any of the Examples described below. For example, the amount can be effective to provide a difference of 30% or less between the amount of active released during in vitro dissolution after exposure to storage conditions of at least one month at 25° C. and 60% relative humidity at a dissolution time point and the amount released at the same dissolution time point during in vitro dissolution conducted prior to exposing the formulation to the storage conditions, while a comparative formulation lacking the stabilizing agent would result in a greater difference in dissolution release following the same storage conditions.

In one aspect, the formulation is an improved formulation for controlled release of a vitamin D compound in the gastrointestinal tract of a subject which ingests the formulation. In one embodiment, the improvement comprises admixing a cellulosic stabilizing agent into a formulation for controlled release of a vitamin D compound in the gastrointestinal tract of a subject which ingests the formulation. In another embodiment, the improvement comprises an effective amount of a cellulosic compound admixed into a formulation for controlled release of a vitamin D compound in the gastrointestinal tract of a subject which ingests the formulation to provide an advantageous degree of stability as described herein, e.g. with respect to the table immediately above or consistent with any of the examples described below. For example, the amount can be effective to provide a difference of 30% or less between the amount of active released during in vitro dissolution after exposure to storage conditions of at least one month at 25° C. and 60% relative humidity at a dissolution time point and the amount released at the same dissolution time point during in vitro dissolution conducted prior to exposing the formulation to the storage conditions, while a comparative formulation lacking the stabilizing agent would result in a greater difference in dissolution release following the same storage conditions.

The stabilizing agents can include cellulose compounds. Examples of cellulose compounds and stabilizing agents for use in the stabilized formulations of the disclosure can include, but are not limited to, celluloronic acid, carboxy methyl cellulose, ethyl cellulose, hydroxyl ethyl cellulose, hydroxyl propyl cellulose, hydroxyl propyl methyl cellulose, methylcellulose, polyanionic cellulose, and combinations thereof. Also contemplated are one or more of poloxamers (e.g., polaxamer 407), poly (ethylene oxide) polymers (e.g., Dow's POLYOX polymers), povidones, and fumed silicas (e.g., AEROSIL 200, Evonik Industries AG, Essen, Germany). The stabilizer, e.g. a cellulosic compound, preferably is present in an amount of at least about 5% of the formulation, based on the total weight of the formulation excluding any additional coatings or shells (wt %). For example, the cellulosic compound can be present in an amount of at least 5 wt % of the formulation, or at least 10 wt % of the formulation, or at least 15 wt % of the formulation, or greater than 5 wt % of the formulation, or greater than 10 wt % of the formulation, or greater than 15 wt % of the formulation. Suitable ranges include 5 wt % to 30 wt %, 10 wt % to 20 wt %, 10 wt % to 15 wt %, 5 wt % to 15 wt %, and 7.5 wt % to 12.5 wt. %. Examples include about 5 wt %, about 6 wt %, about 7 wt %, about 8 wt %, about 9 wt %, about 10 wt %, about 11 wt %, about 12 wt %, about 13 wt %, about 14 wt %, and about 15 wt %. It will be understood that the stabilizing agent referred to herein is an agent that stabilizes the dissolution release profile (and thus also the in vivo release profile) against substantial change over time during storage conditions, e.g. typical shelf storage conditions. Other agents which are known in the art as preservatives for preventing degradation of the active component itself are not intended to be encompassed within the terms “stabilizing agent” and “stabilizer” although such preservatives are also contemplated for use in the formulations of the present invention.

In one class of embodiment, the cellulosic compound is a cellulose ether. Examples of cellulose ethers include, but are not limited to, methylcellulose, hydroxyl propyl methylcellulose, hydroxyl ethyl methylcellulose, hydroxyl ethyl cellulose, hydroxyl propyl cellulose, and combinations thereof.

Hydroxpropyl methylcellulose (HPMC) is particularly contemplated. The HPMC can be characterized by one or more of the following features, which are specifically contemplated individually and in combinations. The % methyoxyl component in the HPMC can be in a range of 19 to 24. The % hydroxypropyl component can be in a range of 7 to 12. The apparent viscosity (2% solution in water at 20° C.) can be at least 50,000 cP, or at least 80,000 cP, or in a range of about 80 to 120,000 cP, or 3000 to 120,000 cP, or 11,000 to 120,000 cP, or 80,000 to 120,000 cP. Particularly, the apparent viscosity (2% solution in water at 20° C.) can be in a range of 80,000 to 120,000 cP. The pH (1% solution in water) can be in a range of 5.5 to 8.0. For example, a suitable hydroxyl propyl methylcellulose having all of the foregoing properties, including an apparent viscosity (2% solution in water at 20° C.) in a range of 80,000 to 120,000 cP, is METHOCEL K100M CR (Dow Wolff Cellulosics, Midland, Mich.).

In one type of embodiment, the cellulosic compound will be insoluble in the matrix formulation at the melt point of the primary components of the matrix, e.g., at 65° C. or in a range of 60° C. to 75° C.

In one type of embodiment, the cellulosic compound will be hydrophilic. The stabilized wax matrix formulation (e.g., Rayaldee®-type) can have the following composition filled into soft OptiShell® plant polysaccharide shells: calcifediol 0.02% of capsule fill by weight, paraffin 20.0% of capsule fill by weight, mineral oil 35.34% of capsule fill by weight, Hypromellose 10.0% of capsule fill by weight, mono- and di-glycerides 22.56% of capsule fill by weight, lauroyl polyoxylglycerides 9.75% of capsule fill by weight, dehydrated alcohol 2.32% of capsule fill by weight, and BHT 0.02% of capsule fill by weight.

The pharmaceutical formulations according to the disclosure comprising one of more of 25-hydroxyvitamin D₂ and 25-hydroxyvitamin D₃ and a cellulosic compound have improved stability compared to formulations lacking a cellulosic compound. In one embodiment, a stabilized formulation according to the disclosure comprises a mixture of an active-loaded lipophilic matrix comprising one or both of 25-hydroxyvitamin D₂ and 25-hydroxyvitamin D₃ and a cellulosic stabilizing agent, wherein the formulation releases an amount of 25-hydroxyvitamin D during in vitro dissolution after exposure to storage conditions of at least one month at 25° C. and 60% relative humidity that varies at any given dissolution time point by 30% or less compared to the amount released at the same dissolution time point during in vitro dissolution conducted on freshly-made product.

Formulations that are not stabilized exhibit changes in the amount of active ingredient released after the composition is stored for a period of time. An unstabilized formulation releases an amount of 25-hydroxyvitamin D following exposure to storage conditions that can vary at a given dissolution time point, for example by more than 30% compared to the amount released at the same dissolution time point during in vitro dissolution conducted on freshly-made product. The changes may be an increase or decrease in the dissolution rate at a given time point, and such changes produce a dissolution profile whose curve is distinct from the shape of the initial dissolution profile. An unstabilized formulation also exhibits different in vivo effects compared to a stabilized formulation according to the disclosure, following storage as described herein, e.g. following 3 months or more of storage at 25° C. and 60% RH. A stabilized formulation demonstrates different clinical pharmacokinetic parameters, such as improved bioavailability, compared to an unstabilized formulation, following storage as described herein, e.g. following 3 months or more of storage at 25° C. and 60% RH. A stabilized formulation according to the disclosure can have a base formulation which is storage unstable, combined with a stabilizing agent which renders the formulation storage stable as described herein.

The matrix that releasably binds and controllably releases the active component can be, for example, a lipophilic matrix, including a wax matrix. A wax matrix can provide a formulation which is solid or semi-solid at room temperature and solid, semi-solid, or liquid at body temperature, preferably semi-solid or liquid at body temperature. In one aspect, the wax matrix comprises a controlled release agent, an emulsifier, and an absorption enhancer.

Examples of controlled release agents suitable for use include, but are not limited to, waxes, including synthetic waxes, microcrystalline wax, paraffin wax, carnauba wax, and beeswax; polyethoxylated castor oil derivatives, hydrogenated vegetable oils, glyceryl mono-, di- or tribehenates; long-chain alcohols, such as stearyl alcohol, cetyl alcohol, and polyethylene glycol; and mixtures of any of the foregoing. Non-digestible waxy substances, such as hard paraffin wax, are preferred.

The controlled release agent can be present in an amount of at least 5 wt % of the stabilized matrix formulation, or greater than about 5 wt % of the formulation. For example, depending on the controlled release agent used, the controlled release agent can comprise at least 5 wt % of the formulation or at least 10 wt % of the formulation, or at least 15 wt % of the formulation, or at least 20 wt % of the formulation, or at least 25 wt % of the formulation, or greater than 5 wt % of the formulation, or greater than 10 wt % of the formulation, or greater than 15 wt % of the formulation, or greater than 20 wt % of the formulation, and or greater than 25 wt % of the formulation. The controlled release agent can be present in an amount 50 wt % or less, 40 wt % or less, 35 wt % or less, or 30 wt % or less. Suitable ranges include 5 wt % to 40 wt %, 10 wt % to 30 wt % and 15 wt % to 25 wt %. Examples include about 15 wt %, about 16 wt %, about 17 wt %, about 18 wt %, about 19 wt %, about 20 wt %, about 21 wt %, about 22 wt %, about 23 wt %, about 24 wt %, and about 25 wt %.

Examples of emulsifiers suitable for use in the stabilized matrix formulation include, but are not limited to, lipophilic agents having an HLB of less than 7, such as mixed fatty acid monoglycerides; mixed fatty acid diglycerides; mixtures of fatty acid mono- and di-glycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan esters including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan esters including sorbitan monostearate, sorbitan sesquioleate; fatty acids and their soaps including stearic acid, palmitic acid, and oleic acid; and mixtures thereof.

A preferred lipoidic agent for use in the stabilized matrix formulation is selected from glycerides and derivatives thereof. Preferred glycerides are selected from the group consisting of medium or long chain glycerides, caprylocaproyl macrogolglycerides, and mixtures thereof. Preferred medium chain glycerides include, but are not limited to, medium chain monoglycerides, medium chain diglycerides, caprylic/capric triglyceride, glyceryl monolaurate, glyceryl monostearate, caprylic/capric glycerides, glycerylmonocaprylate, glyceryl monodicaprylate, caprylic/capric linoleic triglyceride, and caprylic/capric/succinic triglyceride.

Monoglycerides having a low melting point are preferred for making the stabilized matrix formulation. Preferred monoglycerides include but are not limited to, glyceryl monostearate, glyceryl monopalmitate, glyceryl monooleate, glyceryl monocaprylate, glyceryl monocaprate, glyceryl monolaurate, etc., preferably glycerol monostearate (GMS). GMS is a natural emulsifying agent. It is oil soluble, but poorly soluble in water. GMS has an HLB value of 3.8. The lipophilic emulsifier can be present in an amount in a range of about 10 wt % to about 40 wt %, or about 20 wt % to about 25 wt %, for example. Other examples include about 20 wt %, about 21 wt %, about 22 wt %, about 23 wt %, about 24 wt %, and about 25 wt %.

Examples of suitable absorption enhancers for use in the stabilized matrix formulation include, but are not limited to, caprylocaproyl macrogolglycerides such as polyethylene glycosylated glycerides, also known as polyglycolized glycerides or PEGylated glycerides. PEGylated glycerides which may be employed in the composition include, but are not limited to, mixtures of monoglycerides, diglycerides, and triglycerides and monoesters and diesters of polyethylene glycol, polyethylene glycosylated almond glycerides, polyethylene glycosylated corn glycerides, and polyethylene glycosylated caprylic/capric triglyceride. The absorption enhancer can have an HLB value from 13 to 18, or from 13 to 15.

One preferred absorption enhancer is known under the trade name GELUCIRE (Gattefossé Corporation, Paramus, N.J., USA). GELUCIRE is a well-known excipient which is a family of fatty acid esters of glycerol and PEG esters, also known as polyglycolized glycerides. GELUCIRE is used in various applications including preparing sustained release pharmaceutical compositions. GELUCIRE compounds are inert, semi-solid waxy materials which are amphiphilic and are available with varying physical characteristics such as melting point, HLB, and solubilities in various solvents. They are surface active in nature and disperse or solubilize in aqueous media forming micelles, microscopic globules or vesicles. They are identified by their melting point/HLB value. The melting point is expressed in degrees Celsius. One or a mixture of different grades of GELUCIRE excipient may be chosen to achieve the desired characteristics of melting point and/or HLB value. A preferred GELUCIRE composition is GELUCIRE 44/14, a mixture of lauroyl macrogolglycerides and lauroyl polyoxylglycerides that has a melting point of 44° C. and a HLB of 14. The absorption enhancer can be present in an amount of about 5 wt % to about 20 wt %, or about 8 wt % to about 15 wt %, for example. Other examples include about 8 wt %, about 9 wt %, about 10 wt %, about 11, wt % about 12 wt %, about 13 wt %, about 14 wt %, and about 15 wt %.

The low melting points of the wax matrix provide a means of incorporating the pharmaceutically active ingredients, e.g. the vitamin D compound such as 25-hydroxyvitamin D₂, 25-hydroxyvitamin D₃, or both, at temperatures from about 0° C. to about 50° C. above the melting point of the wax matrix and then filling the melt (solution and/or dispersion) in suitable capsules. The capsules can be of any variety that is compatible with the temperature of the melt fill, including soft or hard gelatin capsules, and animal or vegetable gelatin capsules. The melt solidifies inside the capsules upon cooling to room temperature.

In one aspect, the stabilized matrix formulation may further comprise an oily vehicle for the 25-hydroxyvitamin D₂ and/or 25-hydroxyvitamin D₃. Any pharmaceutically-acceptable oil can be used. Examples include animal (e.g., fish), vegetable (e.g., soybean), and mineral oils. The oil preferably will readily dissolve the 25-hydroxyvitamin D compound used. Preferred oily vehicles include non-digestible oils, such as mineral oils, particularly liquid paraffins, and squalene. The oily vehicle can be present at a concentration in a range about 10 wt % to about 50 wt % of the formulation, or about 15 wt % to about 45 wt %, or about 20 wt % to about 40 wt %, or about 30 wt % to about 40 wt %, for example. In one type of embodiment, a suitable liquid paraffin can be characterized by one or more of the following parameters: specific gravity about 0.88 to 0.89; kinematic viscosity (40° C.) about 64 cSt to about 70 cSt; molecular weight 424; % paraffinic hydrocarbons about 59; and pour point −24° C. The ratio between the wax matrix and the oily vehicle can be optimized in order to achieve the desired rate of release of the vitamin D compound. Thus, if a heavier oil component is used, relatively less of the wax matrix can be used, and if a lighter oil component is used, then relatively more wax matrix can be used.

The stabilized controlled release compositions in accordance with the disclosure preferably are designed to contain concentrations of 25-hydroxyvitamin D₂ and/or 25-hydroxyvitamin D₃ of 1 to 1000 g per unit dose, for example, and are prepared in such a manner as to effect controlled or substantially constant release of the 25-hydroxyvitamin D₂/25-hydroxyvitamin D₃, optionally into the ileum of the gastrointestinal tract, of humans or animals over an extended period of time. Example dosages include 1 g to 1000 g per unit dose, 1 g to 600 g, 1 g to 400 g, 1 g to 200 g, 1 g to 100 g, 5 g to 90 g, 30 g to 80 g, 20 g to 60 g, 30 g to 60 g, 35 g to 50 g, g to 50 g, and 10 g to 25 g, for example 20 g, 25 g, 30 g, 40 g, 50 g, 60 g, 70 g, 80 g, 90 g, and 100 g.

The foregoing formulations can optionally exist as a hard capsule formulation. Thus, another aspect of the disclosure herein is an extended-release hard capsule formulation containing a 25-hydroxyvitamin D compound, e.g. for oral administration. The formulation can optionally also have delayed release characteristics. In any of the methods described herein, the 25-hydroxyvitamin D compound(s) can be administered in the form of the hard capsule formulation as described herein.

A hard capsule formulation of 25-hydroxyvitamin D can be used to treat any patient in need of 25-hydroxyvitamin D. Patients in need of vitamin D supplementation include healthy subjects and subjects at risk for or having vitamin D insufficiency or deficiency, for example, subjects with stage 1, 2, 3, 4 or 5 CKD; infants, children and adults that do not drink vitamin D fortified milk (e.g. lactose intolerant subjects, subjects with milk allergy, vegetarians who do not consume milk, and breast fed infants); subjects with rickets; subjects with dark skin (e.g., in the U.S., 42% of African American women between 15 and 49 years of age were vitamin D deficient compared to 4% of white women); the elderly (who have a reduced ability to synthesize vitamin D in skin during exposure to sunlight and also are more likely to stay indoors); institutionalized adults (who are likely to stay indoors, including subjects with Alzheimer's disease or mentally ill); subjects who cover all exposed skin (such as members of certain religions or cultures); subjects who always use sunscreen (e.g., the application of sunscreen with an Sun Protection Factor (SPF) of 8 reduces production of vitamin D by 95%, and higher SPFs may further reduce cutaneous vitamin D production); subjects with fat malabsorption syndromes (including but not limited to cystic fibrosis, cholestatic liver disease, other liver disease, gallbladder disease, pancreatic enzyme deficiency, Crohn's disease, inflammatory bowel disease, sprue or celiac disease, or surgical removal and/or bypass of part or all of the stomach and/or intestines); subjects with inflammatory bowel disease; subjects with Crohn's disease; subjects who have had small bowel resections; subjects with gum disease; subjects taking medications that increase the catabolism of vitamin D, including phenytoin, fosphenytoin, phenobarbital, carbamazepine, and rifampin; subjects taking medications that reduce absorption of vitamin D, including cholestyramine, colestipol, orlistat, mineral oil, and fat substitutes; subjects taking medications that inhibit activation of vitamin D, including ketoconazole; subjects taking medications that decrease calcium absorption, including corticosteroids; subjects with obesity (vitamin D deposited in body fat stores is less bioavailable); subjects with osteoporosis and/or postmenopausal women. According to the Institute of Medicine's report on the Dietary Reference Intakes for vitamin D, food consumption data suggest that median intakes of vitamin D for both younger and older women are below current recommendations; data suggest that more than 50% of younger and older women are not consuming recommended amounts of vitamin D.

In other aspects, the compositions and methods of the invention are useful for prophylactic or therapeutic treatment of vitamin D-responsive diseases, i.e., diseases where vitamin D, 25-hydroxyvitamin D or active vitamin D (e.g., 1,25-dihydroxyvitamin D) prevents onset or progression of disease, or reduces signs or symptoms of disease. Such vitamin D-responsive diseases include cancer (e.g., breast, lung, skin, melanoma, colon, colorectal, rectal, prostate and bone cancer). 1,25-dihydroxyvitamin D has been observed to induce cell differentiation and/or inhibit cell proliferation in vitro for a number of cells. Vitamin D-responsive diseases also include autoimmune diseases, for example, type I diabetes, multiple sclerosis, rheumatoid arthritis, polymyositis, dermatomyositis, scleroderma, fibrosis, Grave's disease, Hashimoto's disease, acute or chronic transplant rejection, acute or chronic graft versus host disease, inflammatory bowel disease, Crohn's disease, systemic lupus erythematosis, Sjogren's Syndrome, eczema and psoriasis, dermatitis, including atopic dermatitis, contact dermatitis, allergic dermatitis and/or chronic dermatitis. Vitamin D-responsive diseases also include other inflammatory diseases, for example, asthma, chronic obstructive pulmonary disease, polycystic kidney disease, polycystic ovary syndrome, pancreatitis, nephritis, hepatitis, and/or infection. Vitamin D-responsive diseases have also been reported to include hypertension and cardiovascular diseases. Thus, the invention contemplates prophylactic or therapeutic treatment of subjects at risk of or suffering from cardiovascular diseases, for example, subjects with atherosclerosis, arteriosclerosis, coronary artery disease, cerebrovascular disease, peripheral vascular disease, myocardial infarction, myocardial ischemia, cerebral ischemia, stroke, congestive heart failure, cardiomyopathy, obesity or other weight disorders, lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes mellitus, hyperinsulinemia, impaired glucose tolerance, insulin resistance, diabetic complication including neuropathy, nephropathy, retinopathy, diabetic foot ulcer and cataracts), and/or thrombosis.

Diseases which can benefit from a modulation in the levels of vitamin D compounds, include, but are not limited to: (i) in the parathyroid—hypoparathyroidism, pseudohypo-parathyroidism, secondary hyperparathyroidism; (ii) in the pancreas—diabetes; (iii) in the thyroid—medullary carcinoma; (iv) in the skin—psoriasis; wound healing; (v) in the lung—sarcoidosis and tuberculosis; (vi) in the kidney—chronic kidney disease, hypophosphatemic VDRR, vitamin D dependent rickets; (vii) in the bone—anticonvulsant treatment, fibrogenisis imperfecta ossium, osteitis fibrosa cystica, osteomalacia, osteoporosis, osteopenia, osteosclerosis, renal osteodytrophy, rickets; (viii) in the intestine—glucocorticoid antagonism, idopathic hypercalcemia, malabsorption syndrome, steatorrhea, tropical sprue; and (ix) autoimmune disorders.

In embodiments, the disease that benefits from a modulation in the levels of vitamin D compounds are selected from cancer, dermatological disorders (for example, psoriasis), parathyroid disorders (for example, hyperparathyroidism and secondary hyperparathyroidism), bone disorders (for example, osteoporosis) and autoimmune disorders. In embodiments, the hard capsule 25-hydroxyvitamin D formulation can be used in treatment of SARS-CoV-2 infection. In embodiments, the hard capsule formulation can be used in treatment secondary hyperparathyroidism in patients having Chronic Kidney disease, optionally Stage 3, 4, or 5 CKD, optionally Stage 3 or 4 CKD, optionally Stage 5 CKD, and optionally patients on hemodialysis. A hard capsule formulation of 25-hydroxyvitamin D can be used in lowering serum iPTH levels.

Without limitation, the formulations and dosage forms described herein may be used to treat patients having chronic kidney disease (stages 3, 4 or 5) and secondary hyperparathyroidism as well as treating vitamin D insufficiency and symptoms related to COVID-19. The formulations are particular useful in controlling release of calcifediol over an extended period of time to achieve efficacious reduction of parathyroid hormone in CKD patients and/or to treat patients infected with SARS-CoV-2. The hard capsule formulations are also effective in preventing early release of API in the first two hours after administration. The present invention thus comprises an extended release dosage form of calcifediol which has an in vitro dissolution profile under two phase acidic/neutral conditions, e.g. 2 hours a pH 1.2 or 1.5, then with transfer to a buffered aqueous medium at pH 6.5, or 6.8 wherein no more than about 5%, or about 4%, or about 3%, or about 2%, or about 1% of calcifediol is released during the first, two-hour period. In one aspect, the dissolution method can be 2 hours at pH 1.5, then with transfer to pH 6.5 buffered medium. In another aspect, the dissolution method can be 2 hours at pH 1.2, then with transfer to pH 6.8 buffered medium. For example, the dissolution method can be according to USP-NF method <711> using Apparatus 1 or 2 and Method B (1000 mL of 0.1N HCl at 37° C. for 2 hours, drain and then add 1000 mL of pH 6.8 phosphate buffer), optionally Apparatus 2. Thereafter, in a pH 6.8 buffered medium, the release of calcifediol can be up to about 40% or 36% at 4 hours (measured from the start of the 2-phase dissolution testing procedure), at least 60 or 62% at 6 hours, and at least 80 or 84% at 8 hours. In embodiments, the dosage form can be a capsule, optionally a hard capsule. The dissolution conditions can be standard conditions as further described herein.

One type of hard capsule formulation has release-modifying agents including a lipophilic (optionally waxy) fill, emulsifiers, and an absorption enhancer, e.g. same or similar to the wax-based matrix formulations described above, or omitting wax and including higher concentrations of other lipophilic release agents instead. The matrix can be solid or semi-solid at both room temperature and at the normal temperature of the human body. It starts releasing slowly and in a substantially constant fashion, controlling release of the active for a period of at least 4 hours, or at least 8 hours, or at least 10 hours, or at least 12 hours, optionally in a range of 4 to 24 hours, or 6 to 20 hours, or 8 to 18 hours, or 10 to 16, hours, or about 12 hours. The release mechanism can be governed by mechanical erosion and/or gradual disintegration, into the contents of the lumen of the lower small intestine and/or colon, for example.

The hard capsule shell can be made of any suitable composition, including hard gelatin capsules and hydroxypropylmethyl cellulose. HPMC capsules can be optionally modified with other agents, e.g. a gelatinizing agent or gelling aid. The hard capsule shell can include about 0.01 to about 10% by weight of a gelatinizing agent. The gelatinizing agent can include gellan gum.

U.S. Pat. Nos. 5,264,223 and 5,431,917 describe capsules produced by the use of HPMC with a gelatinizing agent such as carrageenan. The production of such capsules were claimed to occur under similar temperature setting as that of gelatin capsules. Shionogi Qualicaps Co. (Japan) produces a HPMC capsule containing carrageenan as a gelling aid (e.g. kappa- and/or iota-carrageenans) and potassium chloride as gelation promoter. U.S. Pat. No. 6,410,050 B1 describes cellulose capsules (including HPMC) containing pectin and glycerin. U.S. Pat. No. 6,517,865 B2 describes HPMC capsules with hydrocolloids such as gellan gum and sequestering agents (such as EDTA, sodium citrate, and citric acid). For example, it describes a capsule material having 90 to 99.98% by weight of at least one cellulose ether having a water content of 2 to 10% and a viscosity of 3 to 15 cps measured in a 2% aqueous solution at 20° C.; 0.01 to 5% by weight of gellan gum; and 0.01 to 8% by weight of a sequestering agent selected from the group consisting of EDTA, sodium citrate, citric acid and combinations thereof. For example, it is contemplated to use a HPMC capsule containing at least one cellulose ether, optionally HPMC, having a water content of 2 to 10% and a viscosity of 3 to 15 cps measured in a 2% aqueous solution at 20° C. and a gelling agent, optionally gellan gum, in an amount of about 0.01 to about 10% by weight, or about 1% to about 8%, or about 2% to about 7%, or about 4% to about 6%, or about 5% by weight. Such gelatinized HPMC capsules are believed to provide a slower rupture or disintegration time, e.g. in the stomach, compared to HPMC capsules without a gelatinizing aid. In addition or in the alternative, the HPMC capsules can comprise an enteric coating to retard or prevent dissolution or disintegration of the capsule shell in the gastric environment. Enteric coating materials which resist dissolution in acidic media and dissolve in neutral and alkaline media are known and include, for example, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate trimellitate, sodium alginate, zein, and a coating solution including a mixture of ethylcellulose, medium chain triglycerides, oleic acid, sodium alginate, and stearic acid. A hard capsule without a gelatinizing agent or with small percentages (e.g. 0-4% w/w) thereof may optionally be enteric coated to achieve minimal to zero release in the 0-2 hour time period after dosing.

The composition comprising the 25-hydroxyvitamin D compound in the hard gelatin shell can include be any one described herein, for example a solid or semi-solid composition, optionally a wax matrix. The amount of wax can be about 20 wt. % to about 36 wt. % based on the weight of the solid or semi-solid composition. The wax of the wax matrix can include a non-digestible wax, optionally paraffin wax. The composition comprising the 25-hydroxyvitamin D compound can further include an oily vehicle, optionally in an amount of about 25 wt. % to about 41 wt. % based on the weight of the solid or semi-solid composition. The oily vehicle can include or consist of a non-digestible oil, optionally mineral oil. The composition including the 25-hydroxyvitamin D compound can further include stabilizing agent, optionally in an amount in a range of about 2 wt. % to about 18 wt. % based on the weight of the solid or semi-solid composition. The the stabilizing agent can include a cellulose ether, for example hydroxypropyl methylcellulose. The composition comprising the 25-hydroxyvitamin D compound can further include an emulsifier, e.g. in an amount in a range of about 10 wt. % to about 26 wt. % based on the weight of the solid or semi-solid composition. The emulsifier can include mono- and diglyceryl esters of long chain, saturated and unsaturated fatty acids, for example. The composition comprising the 25-hydroxyvitamin D compound can further include an absorption enhancer, optionally in an amount in a range of about 3 wt. % to about 17 wt. % based on the weight of the solid or semi-solid composition. The absorption enhancer can include or consist of fatty acid esters of glycerol and PEG esters, optionally lauroyl polyoxylglycerides. The composition comprising the 25-hydroxyvitamin D compound can further include a solvent for the 25-hydroxyvitamin D, optionally in an amount in a range of about 0.2 wt. % to about 6 wt. % based on the weight of the solid or semi-solid composition. The solvent can include or consist of an alcohol, optionally ethanol. The hard capsule dosage form can include the 25-hydroxyvitamin D compound in an amount in a range of about 0.1 μg to about 2 mg, for example. The 25-hydroxyvitamin D compound can include or consist of 25-hydroxyvitamin D₃. The dosage form can include 6 μg to 500 μg bioavailable 25-hydroxyvitamin D, for example. The hard capsule dosage form can be used to treat secondary hyperparathyroidism in a patient having stage 3, 4 or 5 Chronic Kidney Disease.

The calcifediol hard capsule formulation can be prepared by any suitable method, including filling a capsule shell with a flowable material, or filling a capsule shell with a mass or slug of solid or semi-solid material, or enrobing or coating a solid or semisolid mass with a shell composition, for example. The size of the hard capsule can be adjusted depending upon the particular fill ratios of the paraffin and the other excipients, e.g. from size 3 to size 4, to further control the release of the drug.

Provided in the table below are example HPMC hard capsule formulations of 25-hydroxyvitamin D with varied percentages of excipients (all percentages by weight, based on the weight of the fill material in the capsule).

| | ~0% | 0% | 0% | 10% | 20% | 30% | 40% |
|---|---|---|---|---|---|---|---|
| | P. wax | P. wax | P. wax | P. wax | P. wax | P. wax | P. wax |
| Material | % Cap | % Cap | % Cap | % Cap | % Cap | % Cap | % Cap |
| calcifediol | 0.0176% | 0.0176% | 0.0176% | 0.0176% | 0.0176% | 0.0176% | 0.0176% |
| paraffin | 0-2% | 0.00% | 0.00% | 10.00% | 20.00% | 30.00% | 40.00% |
| mineral oil | 45.34% | 35.34% | 55.34% | 45.34% | 35.34% | 25.34% | 15.34% |
| hypromellose K100 | 10-15% | 10.00% | 10.00% | 10.00% | 10.00% | 10.00% | 10.00% |
| mono-and di-glycerides | 22-28% | 41.5% | 22.55% | 22.55% | 22.55% | 22.55% | 22.55% |
| lauroyl polyoxylglycerides | 14.75% | 10.75% | 9.75% | 9.75% | 9.75% | 9.75% | 9.75% |
| dehydrated ethanol | 2.32% | 2.32% | 2.32% | 2.32% | 2.32% | 2.32% | 2.32% |
| BHT | 0.02% | 0.02% | 0.02% | 0.02% | 0.02% | 0.02% | 0.02% |
| Total | 100.00% | 100.00% | 100.00% | 100.00% | 100.00% | 100.00% | 100.00% |

A design of experiments (DOE) study was carried out, varying paraffin wax between 20-40% by weight of the formulation (not including the shell material), the lauroyl polyoxylglycerides from 4.75-14.75%, the mono- and di-glycerides between 22.5-12.5% and HPMC between 6-14%. The mineral oil was kept constant in all formulations as 30%. From this DOE, it was found that to achieve a slower in vitro release profile than the Rayaldee® ERC formulation, the paraffin wax percentages can be greater than >35% and the lauroyl polyoxylglycerides around 4.75%.

The table below provides examples of additional wax-based hard capsule formulations, a Rayaldee-®-type soft capsule formulation (Reference) with a vegetable-based capsule shell, and modified wax-based soft vegetable-based capsule formulations. The soft capsules can be OptiShell® vegetable-based capsules, containing modified starch and iota-carrageenan, for example.

| | | | Modified | Modified | | |
|---|---|---|---|---|---|---|
| | | Rayaldee ®- | wax-based | wax-based | HPMC | HPMC |
| | | type soft | soft | soft | Hard | Hard |
| | | capsule | capsule | capsule | capsule | capsule |
| | | (Reference) | (Slow) | (Fast) | (size 3) | (size 4) |
| Excipient | Function | Reference | Test 2 | Test 1 | Test 3 | Test 4 |
| calcifediol | 25-hydroxy- | 0.0176% | 0.0176% | 0.0176% | 0.0176% | 0.0194% |
| | vitamin | | | | | |
| | D active | | | | | |
| paraffin wax | control | 20.00% | 39.00% | 5.00% | 28.00% | 19.95% |
| | release | | | | | |
| | agent | | | | | |
| mineral oil | carrier | 35.34% | 30.34% | 45.34% | 27.39% | 35.26% |
| hypromellose | stabilizer | 10.00% | 10.00% | 10.00% | 10.00% | 9.98% |
| mono & | emulsifier | 22.55% | 13.55% | 22.55% | 20.50% | 22.50% |
| diglycerides | | | | | | |
| lauroyl | absorption | 9.75% | 4.75% | 14.75% | 11.75% | 9.73% |
| polyoxyl | enhancer | | | | | |
| glycerides | | | | | | |
| dehydrated | solvent | 2.32% | 2.32% | 2.32% | 2.32% | 2.54% |
| ethanol | | | | | | |
| BHT | antioxidant | 0.02% | 0.02% | 0.02% | 0.02% | 0.02% |
| Total | — | 100.00% | 100.00% | 100.00% | 100.00% | 100.00% |

Described in the table below is another hard capsule formulation of 25-hydroxyvitamin D, with a gelatinized HPMC capsule shell. Gellan gum is a hydrophilic polymer and has similar properties to carrageenan used in the vegetable capsule shells of the Reference soft capsule formulation described above. The gelatinized HPMC capsule has a slower rupture/disintegration time in the stomach than non-gelatinized HPMC capsules.

| Fill Material | % of fill by weight | mg/Cap |
|---|---|---|
| calcifediol | 0.0194% | 0.03 |
| paraffin | 27.95% | 43.32 |
| mineral oil | 32.26% | 50 |
| hypromellose k100 | 9.98% | 15.47 |
| mono-and di-glycerides | 17.5% | 27.13 |
| lauroyl polyoxylglycerides | 9.73% | 15.08 |
| dehydrated ethanol | 2.54% | 3.94 |
| BHT | 0.02% | 0.03 |
| total | 100% | 155 |
| Shell Material | % of shell by weight | mg/Cap |
| hypromellose | qsp100 | 35.283 |
| gellan gum | 5 | 1.9 |
| titanium dioxide | 2 | 0.76 |
| Organic colorant | 0.15 | 0.057 |
| Total | 100 | 38 |

The composition can be filled in size 4 gelatinized HPMC capsule shells, e.g. HPMC capsules containing gellan gum.

Thus, another aspect of the disclosure herein is a gelatinized HPMC hard capsule formulation of 25-hydroxyvitamin D. The formulation can comprise 0.1 μg to about 2 mg of a 25-hydroxyvitamin D compound per unit dose, optionally 25-hydroxyvitamin D₂ and/or 25-hydroxyvitamin D₃. The amount of 25-hydroxyvitamin D compound can further be in a range of about 1 μg to about 1 mg, or about 10 μg to about 900 μg, or about 20 μg to about 600 μg, or about 30 μg to about 300 μg, or about 60 μg to about 300 μg, for example about 20 μg, or about 25 μg, or about 30 μg, or about 40 μg, or about 50 μg, or about 60 μg, or about 70 μg, or about 80 μg, or about 200 μg, or about 300 μg, or about 600 μg, or about 900 μg. The formulation can include about 20 wt. % to about 36 wt. % of a wax, optionally a non-digestible wax, e.g. paraffin wax, based on the total weight of the fill material in the hard capsule shell. The amount of wax can further be in a range of about 22 wt. % to about 34 wt. %, or about 24 wt. % to about 32 wt. %, or about 26 wt. % to about 30 wt. %, for example about 25 wt. %, about 26 wt. %, about 27 wt. %, about 28 wt. %, about 29 wt. %, about 30 wt. %, about 31 wt. %, about 32 wt. %, or about 33 wt. %. The formulation can include about 25 wt. % to about 41 wt. % of an oily vehicle, optionally one described above, e.g. a non-digestible oil, e.g. mineral oil, based on the total weight of the fill material in the hard capsule shell. The amount of oily vehicle can further be in a range of about 27 wt. % to about 39 wt. %, or about 29 wt. % to about 37 wt. %, or about 31 wt. % to about 35 wt. %, for example about 29 wt. %, about 30 wt. %, about 31 wt. %, about 32 wt. %, about 33 wt. %, about 34 wt. %, about 35 wt. %, about 36 wt. %, or about 37 wt. %. The formulation can include about 2 wt. % to about 18 wt. % of a stabilizing agent, optionally one described above, e.g. a cellulose ether, e.g. hypromellose, based on the total weight of the fill material in the hard capsule shell. The amount of stabilizing agent can further be in a range of about 4 wt. % to about 16 wt. %, or about 6 wt. % to about 14 wt. %, or about 8 wt. % to about 12 wt. %, for example about 5 wt. %, about 6 wt. %, about 7 wt. %, about 8 wt. %, about 9 wt. %, about 10 wt. %, about 11 wt. %, about 12 wt. %, or about 13 wt. %. The formulation can include about 10 wt. % to about 26 wt. % of an emulsifier, optionally one described above, e.g. mixtures including mono- and diglyceryl esters of long chain, saturated and unsaturated fatty acids, e.g. mono- and di-glycerides NF, based on the total weight of the fill material in the hard capsule shell. The amount of emulsifier can further be in a range of about 12 wt. % to about 24 wt. %, or about 14 wt. % to about 22 wt. %, or about 16 wt. % to about 20 wt. %, for example about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, or about 23 wt. %. The formulation can include about 3 wt. % to about 17 wt. % of an absorption enhancer, optionally one described above, e.g. fatty acid esters of glycerol and PEG esters, e.g. lauroyl polyoxylglycerides (44/14) based on the total weight of the fill material in the hard capsule shell. The amount of absorption enhancer can further be in a range of about 5 wt. % to about 15 wt. %, or about 7 wt. % to about 13 wt. %, or about 9 wt. % to about 11 wt. %, for example about 6 wt. %, about 7 wt. %, about 8 wt. %, about 9 wt. %, about 10 wt. %, about 11 wt. %, about 12 wt. %, or about 13 wt. %. The 25-hydroxyvitamin D active can be dissolved in an alcohol carrier, e.g. ethanol, which is present in the formulation in an amount of about 0.2 wt. % to about 6 wt. %, or about 0.5 wt. % to about 5 wt. %, or about 1 wt. % to about 4 wt. %, or about 2 wt. % to about 4 wt. %, for example about 1.5 wt. %, or about 2.0 wt. % or about 2.5 wt. %, or about 3 wt. %, or about 3.5 wt. %, or about 4 wt. %. The formulation can include a small amount of a preservative, e.g. an antioxidant, e.g. BHT, e.g. in a range of about 0.005 wt. % to about 1 wt. %, or about 0.01 wt. % to about 0.05 wt. %, e.g. about 0.02 wt. %

In an alternative hard capsule formulation type, the wax can be omitted and, for example, the concentration of emulsifier and/or absorption enhancer increased. The formulation can comprise 0.1 μg to about 2 mg of a 25-hydroxyvitamin D compound per unit dose, optionally 25-hydroxyvitamin D₂ and/or 25-hydroxyvitamin D₃. The amount of 25-hydroxyvitamin D compound can further be in a range of about 1 μg to about 1 mg, or about 10 μg to about 900 μg, or about 20 μg to about 600 μg, or about 30 μg to about 300 μg, or about 60 μg to about 300 μg, for example about 20 μg, or about 25 μg, or about 30 μg, or about 40 μg, or about 50 μg, or about 60 μg, or about 70 μg, or about 80 μg, or about 200 μg, or about 300 μg, or about 600 μg, or about 900 μg. The formulation can include about 25 wt. % to about 50 wt. % of an oily vehicle, optionally one described above, e.g. a non-digestible oil, e.g. mineral oil, based on the total weight of the fill material in the hard capsule shell. The amount of oily vehicle can further be in a range of about 25 wt. % to about 45 wt. %, or 27 wt. % to about 45 wt. %, or 27 wt. % to about 39 wt. %, or about 29 wt. % to about 37 wt. %, or about 31 wt. % to about 35 wt. %, for example about 30 wt. %, about 32 wt. %, about 34 wt. %, about 36 wt. %, about 38 wt. %, about 40 wt. %, about 42 wt. %, about 44 wt. %, or about 46 wt. %. The formulation can include about 2 wt. % to about 20 wt. % of a stabilizing agent, optionally one described above, e.g. a cellulose ether, e.g. hypromellose, based on the total weight of the fill material in the hard capsule shell. The amount of stabilizing agent can further be in a range of about 4 wt. % to about 16 wt. %, or about 6 wt. % to about 14 wt. %, or about 8 wt. % to about 12 wt. %, for example about 5 wt. %, about 6 wt. %, about 7 wt. %, about 8 wt. %, about 9 wt. %, about 10 wt. %, about 11 wt. %, about 12 wt. %, or about 14 wt. %. The formulation can include about 15 wt. % to about 45 wt. % of an emulsifier, optionally one described above, e.g. mixtures including mono- and diglyceryl esters of long chain, saturated and unsaturated fatty acids, e.g. mono- and di-glycerides NF, based on the total weight of the fill material in the hard capsule shell. The amount of emulsifier can further be in a range of about 17 wt. % to about 42 wt. %, or 18 wt. % to about 40 wt. %, or 20 wt. % to about 36 wt. %, or 20 wt. % to about 34 wt. %, or about 20 wt. % to about 32 wt. %, or about 20 wt. % to about 30 wt. %, or about 22 wt. % to about 28 wt. %, or for example about 18 wt. %, about 20 wt. %, about 22 wt. %, about 24 wt. %, about 26 wt. %, about 28 wt. %, about 30 wt. %, about 32 wt. %, about 34 wt. %, about 36 wt. %, or about 40 wt. %. The formulation can include about 8 wt. % to about 22 wt. % of an absorption enhancer, optionally one described above, e.g. fatty acid esters of glycerol and PEG esters, e.g. lauroyl polyoxylglycerides (44/14) based on the total weight of the fill material in the hard capsule shell. The amount of absorption enhancer can further be in a range of about 8 wt. % to about 20 wt. %, or about 9 wt. % to about 18 wt. %, or about 10 wt. % to about 16 wt. %, for example about 9 wt. %, about 10 wt. %, about 11 wt. %, about 12 wt. %, about 13 wt. %, about 14 wt. %, about 15 wt. %, or about 16 wt. %. The 25-hydroxyvitamin D active can be dissolved in an alcohol carrier, e.g. ethanol, which is present in the formulation in an amount of about 0.2 wt. % to about 6 wt. %, or about 0.5 wt. % to about 5 wt. %, or about 1 wt. % to about 4 wt. %, or about 2 wt. % to about 4 wt. %, for example about 1.5 wt. %, or about 2.0 wt. % or about 2.5 wt. %, or about 3 wt. %, or about 3.5 wt. %, or about 4 wt. %. The formulation can include a small amount of a preservative, e.g. an antioxidant, e.g. BHT, e.g. in a range of about 0.005 wt. % to about 1 wt. %, or about 0.01 wt. % to about 0.05 wt. %, e.g. about 0.02 wt. %

In another aspect, the 25-hydroxyvitamin D compound(s) can be administered in the form of a formulation as described in international (PCT) application publication WO 2020/044314 A1, including such formulations suitable for dosing to pediatric patients.

Such a formulation can include a vitamin D compound, optionally 25-hydroxyvitamin D or calcifediol, embedded in a polymer network. The polymer can be water-insoluble, and optionally swellable. In embodiments, the formulation can be an extended release formulation, e.g. for oral use.

Such a formulation can include a spheronized pellet formulation comprising a vitamin D compound, optionally 25-hydroxyvitamin D or calcifediol, and a pharmaceutically acceptable excipient. In embodiments, the formulation can be an extended release formulation, e.g. for oral use. In embodiments, the formulation can be a delayed release formulation, or a delayed-sustained release formulation. The spheronized pellets can be disposed in a capsule, which is optionally enteric coated. In the alternative, the pellets can be enteric coated.

Such a formulation can include a vitamin D formulation comprising a vitamin D compound, optionally 25-hydroxyvitamin D or calcifediol, dispersed in a fatty acid glyceride mixture. In embodiments, the formulation can be an extended release formulation, e.g. for oral use.

Such a formulation can include a nano/microparticle formulation comprising a vitamin D compound, optionally 25-hydroxyvitamin D or calcifediol, and a pharmaceutically acceptable excipient. In embodiments, the nano/microparticle formulation can provide extended release of the vitamin D compound, e.g. by using an extended release polymer as an excipient.

Such a formulation can include a lipid microparticle formulation comprising a vitamin D compound, optionally 25-hydroxyvitamin D or calcifediol, and a pharmaceutically acceptable lipid. In embodiments, the formulation can be an extended release formulation, e.g. for oral use.

Such a formulation can include a non-pareil seed formulation comprising a vitamin D compound, optionally 25-hydroxyvitamin D or calcifediol, and a pharmaceutically acceptable excipient. In embodiments, the formulation can be an extended release formulation, e.g. for oral use. In embodiments, the excipient can include an extended release polymer coating.

Such a formulation can include a pharmaceutical composition comprising a vitamin D compound, optionally 25-hydroxyvitamin D or calcifediol, and a pharmaceutically acceptable excipient selected from one or more excipients in the group of an absorption enhancer, a spheronizing aid, a water insoluble polymer, and a binder. In embodiments, the formulation can be an extended release formulation, e.g. for oral use.

Such a formulation can include a spray-congealed lipid vitamin D formulation comprising a vitamin D compound, optionally 25-hydroxyvitamin D or calcifediol, an extended release agent, and a surfactant. In embodiments, the formulation can be an extended release formulation, e.g. for oral use.

The shell compositions of either a hard or soft capsule can be, in a preferred embodiment, compositions which are stable in low pH environments.

The administration of 25-hydroxyvitamin D and treatment of COVID-19 as described herein can be performed in the presence of, or in the absence of, additional therapies. For example, additional treatments for COVID-19 can include one or more compounds in the classes of, antivirals, antimalarials (chloroquine, hydroxychloroquine), and antibiotics. As another example, agents for the potentiation of vitamin D action can be administered, e.g. CYP24 inhibitors which can slow the catabolism of 25-hydroxyvitamin D compounds and 1,25-dihydroxyvitamin D compounds.

Anti-viral agents can include anti-retroviral agents, antibodies against SARS-CoV-2 virus, inhibitors of reverse transcriptase, and can include one or more of maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine and sofobuvir. Other anti-viral agents include peptide based compounds that can target viral/non-viral targets and DNA based compounds that can target viral/non-viral targets.

The anti-viral agent for combination can be, for example, one or more of Retrovir® (3′-azido-3′-deoxypyrimidine, Zidovudine) and 3′-azido-3′-deoxythymidine (AZT) from GlaxoSmithKline, HMD® (2′,3′-dideoxycytidine, Zalcitabine) from Hoffmann-LaRoche, Videx® or VidexEC® (2′,3′dideoxyinosine, Didanosine) from Bristol-Myers-Squibb, Epivir® (Lamivudine) from GlaxoSmithKline, Zerit® (stavudine) from Bristol Myers-Squibb, Viread® (tenofovir DF) from Gilead, Ziagen® (abacavir) from GlaxoSmithKline, Emtriva® (Emtricitabine, FTC) from Gilead Sciences; or non-nucleoside analogues, e.g. Rescriptor® (delavirdine) from Pfizer, Sustiva® (Efavirenz) from Bristol Meyer Squibb, Viramune® (nevirapine, 11-Cyclopropyl-4-methyl-5,11-dihydro-6H-dipyrido[3,2-b:2′,3′-e][1,4]diazepin-6-one) from Boehringer-Ingelheim, trisodium phosphonoformate, ammonium-21-tungstenato-9-antimonate, 1-β-D-ribofuranoxyl-1,2,4-triazole-3-carboxamide; inhibitor of viral or retroviral protease, e.g. inhibitor of viral aspartate protease, e.g. inhibitor of HIV protease, such as Aganerase® (amprenavir) fromGlaxoSmithKline, Reyataz® (atazanavir) from Bristol-Myers Squibb, Lexiva® (fosamprenavir) from GSK, Crixivan® (Indinavir) from Merck & Co.; Viracept® (nelfinavir) from Agouron, Norvir® (Ritonavir) from Abbott; Fortovase® and Invirase® (saquinavir) from Hoffmann-LaRoche; and other compounds such as lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), Adriamycin, KVX-478 from GlaxoWellcome; VX-478 from Vertex; 141W94 from Kissei Pharmaceuticals; AG-1343 from Agouron; KNI-272 from Nippon Mining; U-96988 from Upjohn; BILA-2011 BS(Palinavir) from Boehringer-Ingelheim; compounds preventing virus penetration, such as e.g. polymannoacetate; fusion inhibitors, such as e.g. Fuzeon® (enfuvirtide, T-20) from Hofffmann-LaRoche; or any combination thereof, such as Epzicom® (Abacavir and Lamivudine) from GlaxoKlineSmith, Trizivir® (Abacavir, Lamivudine and Zidovudine) from GlaxoKlineSmith, Truvada® (Emtricitabine and Tenofir DF) from Gilead Sciences, Combivir® (Lamivudine and Zidovudine) from GlaxoKlineSmith, Kaletra® (lopinavir and ritonavir) from Abbott.

Antibacterial agents can include one or more of sulphonamides, amphenicols such as chlorophenicols, spectinomycin, trimethoprim, tigencycline, erythromycin, clarithromycin, azithromycin, linezolid, deoxyclcline, carbapenems such as imipenem, meropenem, aztreonam, ticaracillinclvulnate, piperaciin-tazobactam, cephalosporins, e.g. cefotaxime, ceftriaxone, ceftazidime, and cefepime, gentamicin, tobramycin, and amikacin, ketolides, Quinolones, including axaquin (lomefloxacin), Floxin (ofloxacin), Noroxin (norfloxacin), Tequin (gatifloxacin), Cipro (ciprofloxacin), Avelox (moxifloxacin), Levaquin (levofloxacin), Factive (gemifloxacin), Cinobac (cinoxacin), NegGram (nalidixic acid), Trovan (trovafloxacin), and Zagam (sparfloxacin), flouroquinolones, such as levofloxacin, ciprofloxacin, and oxifloxacin, vancomycine, polymyxins such as polymyxin B and colistin, cephalosporins, carbepenems, macrolides, including axaquin (lomefloxacin), Floxin (ofloxacin), Noroxin (norfloxacin), Tequin (gatifloxacin), Cipro (ciprofloxacin), Avelox (moxifloxacin), Levaquin (levofloxacin), Factive (gemifloxacin), Cinobac (cinoxacin), NegGram (nalidixic acid), Trovan (trovafloxacin), and Zagam (sparfloxacin), Vancomycin, clindamycin, isoniazid, licosamides, mupirocin, rifampin, ethambutol, pyrazinamide, bacitracin, polymixins, sulfonamides, glycopeptide and nitroimidazoles, Ticarcillin, a carboxypenicillin, phenicols, tetracyclines, streptogramins, oxazolidinone, Chloramphenicol, Rifamycins, and penicillins, e.g. penicillin V, penicillin G, procaine penicillin G, benzathine penicillin G, methicillin, oxacillin, cloxacillin, dicloxacillin and flucloxacillin, ampicillin, amoxicillin, propicillin, pheneticillin, azidocillin, clometocillin, and penamecillin or combinations thereof.

In another aspect, the therapy can include administration of a corticosteroid, e.g. one or more in the classes of Class A (hydrocortisone-type) Class B (triamcinolone acetonide type), Class C (betamethasone type), and Class D (hydrocortisone-17-butyrate andclobetasone-17-butyrate type). For example, the corticosteroid can include cortisone, hydrocortisone, methylprednisolone, prednisolone, prednisone, budesonide, fluocinolone, triamcinolone, beclomethasone, dexamethasone, betamethasone, fluticasone, and mometasone. The corticosteroid can be delivered via inhalation, such as prednisone, prednisolone, beclomethasone dipropionate (Beclovent®, Qvar®, and Vanceril®), triamcinolone acetonide (Azmacort®), mometasone (Asthmanex®), budesonide (Pulmocort®, Symbicort®), flunisolide (Aerobid®), fluticasone with salmeterol (Adviar®), and mometasone furoate with formoterol fumarate dihydrate (Dulera®).

The subject can be a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

In various aspects, the subject is infected with SARS-CoV-2. In various aspects, the subject is diagnosed with a SARS-CoV-2 infection. In instances, the diagnosis can based on laboratory testing, irrespective of clinical signs and symptoms. For example, the diagnosis cay be based on positive viral nucleic acid test result on a throat swab sample. In aspects, the subject is clinically-diagnosed with the SARS-CoV-2 infection based on symptoms and exposures only, and not by a viral nucleic acid test. In various aspects, the clinically-diagnosed subject is one which demonstrates the presence of lung imaging features consistent with coronavirus pneumonia. In various instances, the subject is one infected with SARS-CoV-2 and is undiagnosed and otherwise unaware of the infection. In instances, the subject can be asymptomatic.

In various aspects, the patient's baseline serum total 25-hydroxyvitamin D level can be less than about 30 ng/mL, or less than about 20 ng/mL, or in a range of 20 ng/mL to 30 ng/mL, or in a range of about 20 ng/mL to about 25 ng/mL.

In various aspects, the subject exhibits one or more of the following symptoms: fever, fatigue, cough, and/or difficulty breathing. In exemplary instances, the subject exhibits pneumonia or pneumonia-like symptoms. Optionally, the subject has been diagnosed with Coronavirus Disease 2019 (COVID-19). The World Health Organization's definition of a confirmed case of COVID-19 is a person shown by laboratory testing to be infected with SARS-CoV-2, irrespective of clinical signs and symptoms. In various instances, the subject is at least or greater than 50 years old, 60 years old, 70 years old, or 80 years old. In various aspects, the subject has at least one underlying medical comorbidity, including but not limited to cardiovascular disease, diabetes mellitus, obesity, hypertension, chronic lung disease, cancer, chronic kidney disease (CKD). Optionally that subject has Stage 3 or Stage 4 CKD. Optionally, the subject has Stage 5 CKD. Optionally, the patient has an eGFR of at least 15 mL/min/1.73 m², and further optionally eGFR<60 mL/min/1.73 m². In various aspects, the patient is receiving dialysis therapy, e.g., hemodialysis therapy.

The methods are contemplated to include embodiments including any combination of one or more of the additional optional elements, features, and steps further described below (including those shown in the figures), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “comprising” indicates the potential inclusion of other agents, elements, steps, or features, in addition to those specified.

### EXAMPLES

The following examples are provided for illustration and are not intended to limit the scope of the invention.

### Example 1

This is a double-blind randomized, placebo-controlled trial of ERC (commercially available as Rayaldee®) in SARS-CoV-2 patients presenting to a “drive through” testing center.

An objective is to demonstrate that rapid elevation of serum total 25-hydroxyvitamin D to a sufficiently elevated level, or maintenance of a sufficiently high serum total 25-hydroxyvitamin D level, in SARS-CoV-2 infected patients leads to improved outcomes as evidenced by one or more of (1) severity and duration of COVID-19 illness as evidenced by six symptoms (cough, difficulty breathing, fatigue, head-ache, myalgia, and feverishness), using a 4-point NRS (0, absent; 3, severe) recorded twice daily for 14 days or 20 days or 21 days, compared to placebo (Treanor et al., JAMA vol. 283, no. 8, Feb. 23, 2000), (2) a significant decrease in the number of hospital admissions for SARS-CoV-2 infection, compared to placebo, (3) a significant decrease in mortality rates due to SARS-CoV-2 infection, compared to placebo, (4) a significant decrease in the duration of hospital admission for SARS-CoV-2 infection, compared to placebo, and (5) more rapid decline in viral titers over a 2-week period, a 20 day period, or 3-week period, compared to placebo; and (6) more rapid decline in serum anti-SARS-CoV-2 antibody titer over a 2-week period, or 20 day period, or 3-week period, compared to placebo. The elevated serum total 25-hydroxyvitamin D levels are at least 50 ng/mL, or at least 60 ng/mL, or greater than 60 ng/mL. Another objective is to demonstrate that a 900 μg loading dose and 60 μg/day maintenance dose of ERC rapidly and reliably increase and maintain serum total 25-hydroxyvitamin D levels to at least 50 ng/mL compared to placebo.

Another objective is to demonstrate that ERC treatment boosts serum biomarkers of an innate and adaptive immune response in SARS-CoV-2 infected patients. Another objective is to demonstrate that ERC treatment accelerates the acute, subacute, and late inflammation-response-related methylome and transcriptome in peripheral blood mononuclear cells compared to placebo-group SARS-CoV-2 infected patients, the latter performed in bulk and single cell RNA-sequencing in a matched subgroup of ERC-treated and placebo-treated subjects.

An approximately even number of subjects are in ERC and placebo groups. Approximately 83 subjects are in each of the ERC treatment and placebo groups, for a total of approximately 166 subjects.

Inclusion criteria state that participants must be at least 18 years of age and confirmed to have SARS-CoV-2 infection within the last three days. Further, participants i) can be male and female, ii) can be between the ages of 18-70 years, iii) if an ovulating female, must have a negative pregnancy test confirmed by urine sample at enrollment and prior to taking first study drug dose, iv) if an ovulating female, must be willing to use an effective method of birth control during the study (female participants should avoid pregnancy), v) must be willing to limit the use of vitamin D supplements except for normally fortified food products (e.g., milk) during the course of the 2-week study, vi) must demonstrate the ability to comply with all study requirements, and vii) must be without any disease state or physical condition that might impair evaluation of safety or which, in the investigator's opinion, would interfere with study participation.

Exclusion criteria state that subjects who meet any of the following criteria are excluded from the study: those who i) are pregnant, ii) who have been on glucocorticoid medications in the last six months, iii) have a history of hyperparathyroidism, kidney stones, hypercalciuria or hypercalcemia, iv) have a history of a chronic granuloma-forming disease (e.g., sarcoidosis), v) have any surgical or medical condition which might significantly alter the absorption, distribution, metabolism, or excretion of vitamin D or 25-hydroxyvitamin D (e.g., small bowel resection), vi) have ongoing treatment with thiazides or high doses of diuretics, vii) have renal impairment measured as eGFR<60 ml/min/1.73 m² on screening serum creatinine on blood draw, and/or viii) have a serum total calcium >9.8 mg/dL on blood draw, ix) show evidence of existing or impending dehydration, x) are known or suspected to have hypersensitivity to any of the constituents of the study drug, and/or xi) are currently participating in, or have participated in, an interventional/investigational study within 30 days prior to study screening. Any subject that reports signs or symptoms of hypercalcemia during the course of the trial and has a confirmed serum calcium >10.3 mg/dL is removed from the trial and their randomization status disclosed to the responsible investigator(s) and the appropriate healthcare providers alerted. Dosing is suspended if serum total 25-hydroxyvitamin D exceeds 100 ng/mL at Day 7 or Day 14.

Eligible subjects in the treatment arm receive oral ERC (Rayaldee® extended release calcifediol capsules) in a 900 μg loading dose on Day 1, followed by a maintenance dose of 60 μg per day for the subsequent 19 days (Days 2-20). Doses are administered orally at bedtime, or at another time of the day after fasting for at least 3 hours. Following the loading dose, subjects continue to fast for at least 3 hours.

Eligible subjects in the placebo arm receive oral look-alike formulations on the same schedule as the treatment arm.

Concomitant medications are allowed, with the exception of (1) thiazides or high doses of non-thiazide diuretics, (2) medications that could impair the absorption of fat-soluble nutrients, and (3) dietary supplements providing in excess of 1.0 g of elemental calcium per day.

Blood samples, spot urine samples, nasal swab SARS-CoV-2 titer levels and serum anti-SARS-CoV-2 antibody titers are acquired periodically during the study, as described below. Blood samples are assayed for: i) serum total 25-hydroxyvitamin D ii) serum free 25-hydroxyvitamin D, iii) serum intact parathyroid hormone (iPTH; a marker of 25-hydroxyvitamin D insufficiency-driven hyperparathyroidism), iv) LL37, v) three circulating biomarkers of enhanced innate immunity: eotaxin, the monocyte chemotactic protein MCP1 (also known as CCL2) and interleukin-12 (IL-12) a potent inducer of T-cell interferon gamma (IFN-γ) production, vi) serum interleukin-6 (IL-6) as a marker of the adaptive immune response to SARS-COv-2 infection. Harvested peripheral blood neutrophils and mononuclear monocytes (PBMC) are subjected to differential cell counting, immune cell surface marker phenotyping by fluorescent activated cell sorting (FACS) as well as differential whole genome bisulfite DNA sequencing with deconvolution analysis to ascertain the “signature” of unmethylated, transcriptionally-active genes in monocytes and neutrophils. Comparative pre- and serial post-intervention clinical disease severity is assessed by daily scoring. Spot urine samples are analyzed for calcium:creatinine ratio (normal <0.2).

On each day during a dosing period, subjects under treatment record their ability to perform usual activities on a diary card using an 11-point NRS (unable to perform normal activity, 0; fully able to perform normal activity, 10). Subjects under treatment complete a record of their opinion of overall health status including normal, pre-SARS-CoV-2 health on an 11-point NRS (0, worst health and 10, best possible health); following this, they record their assessment of health status at baseline and over a 24-hour period once daily in the evening. Subjects also take and record oral temperature by digital thermometer twice daily. Additional details are described in the tables and schedule below.

TABLE 1
Evaluations

| | |
|---|---|
| Physical | Height, weight and BMI determination at |
| Examination: | screening visit and on Day 1 |
| Vital Sign | Blood pressure and heart rate measured and |
| Measurements: | recorded after the subject has been sitting for at |
| | least 2 minutes prior to any scheduled blood |
| | draws at every visit. |
| Assessment of | On each day during the dosing period, subjects: i) |
| COVID-19 | record twice daily the severity of 6 COVID-19 |
| disease | symptoms (cough, difficulty breathing, fatigue, |
| severity: | head-ache, myalgia, and feverishness) using a 4- |
| | point NRS (0, absent; 3, severe); ii) record once |
| | daily their ability to perform usual activities on a |
| | diary card using an 11-point NRS (unable to |
| | perform normal activity, 0; fully able to perform |
| | normal activity, 10); and, iii) complete once |
| | daily a record of their opinion of overall health |
| | status using an 11-point NRS (0, worst health and |
| | 10, best possible health). |

Blood is drawn on Days 0, 1, 7, 14 and 21 and analyzed as specified in Table 2. Samples also are collected for analyses of biomarkers associated with SARS-CoV-2 infection, host immune status and vitamin D metabolism.

All serum calcium values are adjusted for serum albumin level of <4.0 g/dL. Spot-urine samples are obtained on Days 1, 7, 14 and 21 and analyzed as specified in Table 2. Nasal swab samples are taken on Day −3 to 0, 1, 7, 14 and 21 and analyzed as specified in Table 2.

TABLE 2
List of Laboratory Tests

| | |
|---|---|
| Hematology: | Serum Chemistry: |
| Hematocrit | Albumin |
| Hemoglobin | Alkaline phosphatase |
| Platelet count | Alanine aminotransferase (ALT) |
| Red blood cell count | Anti-SARS-CoV-2 antibody titer |
| White blood cell count | Aspartate aminotransferase (AST) |
| Peripheral blood neutrophils | Blood urea nitrogen |
| Peripheral mononuclear monocytes | Calcium (corrected) |
| Serum b-hCG Pregnancy Test (only for females of | Carbon dioxide |
| child bearing potential, defined as either not | Chloride |
| surgically sterile or not diagnosed as | Creatinine |
| postmenopausal) | Estimated glomerular filtration |
| Biomarkers, PD and PK parameters: | rate (eGFR) |
| Serum 1,25-dihydroxyvitamin D (1,25D) (blinded) | Glucose |
| Serum iPTH | Lactate dehydrogenase |
| Serum total 25-hydroxyvitamin D (25D) (blinded) | Phosphorus |
| Serum calcifediol (25D₃) (blinded) | Potassium |
| Serum 1,25-dihydroxyvitamin D₃ (blinded) | Sodium |
| Serum LL37 | Total bilirubin |
| Serum IL-1β | Direct bilirubin |
| Serum caspase-3 | Total cholesterol |
| Serum eotaxin | Total protein |
| Serum MCP1 (CCL2) | Triglycerides |
| Serum IL12 | Uric acid |
| Serum IL6 | Spot urine chemistry: |
| PBMC for indicators of activated innate and | Calcium |
| adaptive immunity | Creatinine |
| Fluorescent activated cell sorting (FACS): | Nasal swabs: |
| Immune cell surface marker phenotyping | SARS-CoV2 titer |
| Differential whole genome bisulfite DNA | |
| sequencing | |

The study activities are described in additional detail below.

### Screening Period (Days −3 to 0)

### Screening Visit (Visit 1/Days −3 to 0)

The screening visit occurs within 3 days of positive test for SARS-CoV-2 infection. Physical examination (weight, height and BMI) and vital signs assessment are performed. A nasal swab for SARS-CoV-2 infection is obtained. Blood samples are drawn for clinical chemistry (full panel and eGFR determination), hematology, serum iPTH, and serum total 25-hydroxyvitamin D.

### Randomization and Treatment Period (Days 1-20)

### Visit 2 (Day 1)

First morning void spot urine collection is performed (may be taken pre-visit dependent on visit timing). Physical examination (weight, height and BMI) and vital signs assessment are performed. A nasal swab for SARS-CoV-2 infection is obtained. Blood samples are drawn for clinical chemistry (full panel and eGFR determination), hematology, biomarkers, PD and PK parameters (see Table 2), anti-SARS-CoV-2 antibody titer, and blood PBMC and serum for host immune response detection and quantitation. Subjects are randomized to treatment with ERC or placebo.

Subjects assess and record on a diary card at the visit: severity of six COVID-19 symptoms (cough, difficulty breathing, fatigue, headache, myalgia, and feverishness) using a 4-point NRS (0, absent; 3, severe), and oral temperature using a digital thermometer.

Subjects self-administer 900 μg dose of capsules of assigned drug product from “loading dose” bottle after fasting for at least 3 hours. No food is ingested for at least 3 hours after dosing. Dosing is recorded on the diary card.

Subjects assess and record on diary card at bedtime: severity of six COVID-19 symptoms (cough, difficulty breathing, fatigue, head-ache, myalgia, and feverishness) using a 4-point NRS (0, absent; 3, severe), ability to perform usual activities using an 11-point visual analog NRS (0, unable to perform normal activity; 10, fully able to perform normal activity), their opinion of overall health status using an 11-point NRS (0, worst health; 10, best possible health), and oral temperature using a digital thermometer.

### Days 2-6

No study visits or procedures are carried out on Days 2-6. Subjects self-assess and record on diary card in morning: severity of six COVID-19 symptoms (cough, difficulty breathing, fatigue, headache, myalgia, and feverishness) using a 4-point NRS (0, absent; 3, severe), and oral temperature using a digital thermometer. Subjects self-administer a 60 μg dose of capsules from assigned “maintenance dose” bottle each day at bedtime and record the dose in diary. Subjects self-assess and record on diary card at bedtime: severity of six COVID-19 symptoms (cough, difficulty breathing, fatigue, headache, myalgia, and feverishness) using a 4-point NRS (0, absent; 3, severe), ability to perform usual activities using an 11-point NRS (0, unable to perform normal activity; 10, fully able to perform normal activity), their opinion of overall health status using an 11-point NRS (0, worst health; 10, best possible health), and oral temperature using a digital thermometer.

### Visit 3 (Day 7)

Subjects self-assess and record on diary card in morning: severity of six COVID-19 symptoms (cough, difficulty breathing, fatigue, headache, myalgia, and feverishness) using a 4-point NTRS (0, absent; 3, severe), and oral temperature using a digital thermometer.

Activities performed during the site visit include first morning void spot urine collection (may be taken pre-visit dependent on visit timing), vital signs assessment, and nasal swab for SARS-CoV-2 infection. Blood samples are drawn for clinical chemistry (full panel including eGFR determination), biomarkers, PD and PK parameters (see Table 2), anti-COVID-19 antibody titer, and blood PBMC and serum for host immune response detection and quantitation.

Subjects self-administer 60 μg dose of capsules from assigned “maintenance dose” bottle at bedtime and record in diary. Subjects self-assess and record on diary card at bedtime: severity of six COVID-19 symptoms (cough, difficulty breathing, fatigue, headache, myalgia, and feverishness) using a 4-point NRS (0, absent; 3, severe), ability to perform usual activities using an 11-point NRS (0, unable to perform normal activity; 10, fully able to perform normal activity), their opinion of overall health status using an 11-point NRS (0, worst health; 10, best possible health), and oral temperature using a digital thermometer.

### Days 8-13

No study visits or procedures are carried out on Days 8-13. Subjects self-assess and record on diary card in morning: severity of six COVID-19 symptoms (cough, difficulty breathing, fatigue, headache, myalgia, and feverishness) using a 4-point NRS (0, absent; 3, severe), and oral temperature using a digital thermometer. Subjects self-administer a 60 μg dose of capsules from assigned “maintenance dose” bottle each day at bedtime and record the dose in diary. Subjects self-assess and record on diary card at bedtime: severity of six COVID-19 symptoms (cough, difficulty breathing, fatigue, headache, myalgia, and feverishness) using a 4-point NRS (0, absent; 3, severe), ability to perform usual activities using an 11-point NRS (0, unable to perform normal activity; 10, fully able to perform normal activity), their opinion of overall health status using an 11-point NRS (0, worst health; 10, best possible health), and oral temperature using a digital thermometer.

### Visit 4 (Day 14)

Subjects self-assess and record on diary card in morning: severity of six COVID-19 symptoms (cough, difficulty breathing, fatigue, headache, myalgia, and feverishness) using a 4-point NRS (0, absent; 3, severe), and oral temperature using a digital thermometer.

Activities performed during the site visit include first morning void spot urine collection (may be taken pre-visit dependent on visit timing), vital signs assessment, and nasal swab for SARS-CoV-2 infection. Blood samples are drawn for clinical chemistry (full panel including eGFR determination), biomarkers, PD and PK parameters (see Table 2), anti-SARS-Cov-2 antibody titer, and blood PBMC and serum for host immune response detection and quantitation.

Subjects self-administer 60 μg dose of capsules from assigned “maintenance dose” bottle at bedtime and record in diary. Subjects self-assess and record on diary card at bedtime: severity of six COVID-19 symptoms (cough, difficulty breathing, fatigue, headache, myalgia, and feverishness) using a 4-point NRS (0, absent; 3, severe), ability to perform usual activities using an 11-point NRS (0, unable to perform normal activity; 10, fully able to perform normal activity), their opinion of overall health status using an 11-point NRS (0, worst health; 10, best possible health), and oral temperature using a digital thermometer.

### Days 15-20

No study visits or procedures are carried out on Days 15-20. Subjects self-assess and record on diary card in morning: severity of six COVID-19 symptoms (cough, difficulty breathing, fatigue, headache, myalgia, and feverishness) using a 4-point NRS (0, absent; 3, severe), and oral temperature using a digital thermometer. Subjects self-administer a 60 μg dose of capsules from assigned “maintenance dose” bottle each day at bedtime and record the dose in diary. Subjects self-assess and record on diary card at bedtime: severity of six COVID-19 symptoms (cough, difficulty breathing, fatigue, headache, myalgia, and feverishness) using a 4-point NRS (0, absent; 3, severe), ability to perform usual activities using an 11-point NRS (0, unable to perform normal activity; 10, fully able to perform normal activity), their opinion of overall health status using an 11-point NRS (0, worst health; 10, best possible health), and oral temperature using a digital thermometer.

### Follow Up Period, Visit 5 (Day 21)

Subjects self-assess and record on diary card in morning: severity of six COVID-19 symptoms (cough, difficulty breathing, fatigue, headache, myalgia, and feverishness) using a 4-point NRS (0, absent; 3, severe), and oral temperature using a digital thermometer.

Activities performed during the site visit include first morning void spot urine collection (may be taken pre-visit dependent on visit timing), vital signs assessment, and nasal swab for SARS-CoV-2 infection. Blood samples are drawn for chemistry (full panel including eGFR determination) and hematology, biomarkers, PD and PK parameters (see Table 2), anti-SARS-CoV-2 antibody titer, and blood PBMC and serum for host immune response detection and quantitation.

Baseline subject characteristics, including age, sex, race, height, weight, BMI, vital signs (blood pressure and heart rate), and assessment of COVID-19 disease severity are performed at the screening visit scheduled as soon as possible after a subject is verified to be infected. Primary, secondary and exploratory (when available) outcomes are summarized by study arm and follow-up time points, using CONSORT guidelines to present descriptive statistics, including mean, median, standard deviation, quartiles, interquartile range, minimum, and maximum values for subjects within the site, overall and by treatment group. Continuous variables are summarized using means, standard deviations, and quartiles, while categorical variables are summarized using frequencies and percentages.

A two-sample log rank test is used to compare the endpoint of time to symptom resolution between groups. Secondary analyses use Cox-proportional hazards regression models to adjust for covariates (e.g., age, sex, time from symptom onset to randomization). Additional analyses compute a symptom score for each person-day and compute an area under the curve (AUC) which is the sum of these scores over the follow-up period. A two sample t-test is used to compare the AUC between study arms. The proportion of serum total 25-hydroxyvitamin D level consistently ≥50 ng/mL is compared with Fisher's exact test. Secondary analyses use logistic regression to adjust for additional covariates as above.

Analysis of changes from baseline in quantitative outcomes are performed using a linear mixed effects model, including a fixed study arm effect (ERC), a linear time effect (study arm by time interaction effect), baseline subject characteristics as covariates (e.g., age, sex, race) and random subject effects to account for repeated measurements. Differences in changes between groups are estimated using model contrasts.

Treatment group subjects show a significant elevation of serum total 25-hydroxyvitamin D level compared to the placebo group, e.g. to at least >50 ng/mL. Treatment group subjects show significant quantitative reduction in viral titer from initial testing (Day 7, Day 14, Day 20, Day 21), compared to the placebo group. Treatment group subjects also show significantly accelerated and quantitative increase in serum anti-SARS-CoV-2 antibodies (Days 7, 14, 20, 21), compared to the placebo group. Treatment group subjects also show significantly reduced severity in one or more of the six COVID-19 symptoms (cough, difficulty breathing, fatigue, headache, myalgia, and feverishness) on (Days 7, 14, 20, 21), compared to the placebo group. Treatment group patients show acceleration of the acute, subacute, and late inflammation-response-related methylome and transcriptome in peripheral blood mononuclear cells compared to placebo group subjects; the latter performed in bulk and single cell RNA-sequencing in a matched subgroup of treatment group- and placebo-group subjects.

The course of the disease is attenuated. Compared to placebo, hospital admissions for COVID-19 are significantly decreased in the treatment group. Compared to placebo, viral titers are more rapidly decreased over a 2-week period or 20 day period, or 3 week period in the treatment group. The treatment method reduces (compared to placebo) time to resolution of illness, defined as time from study drug initiation to time of alleviation of symptoms, among individuals with SARS-COV-2 infection. Symptom alleviation is considered to occur at the start of the first 24-hour period in which all six symptoms are scored 1 or less (mild or none) and remain so for 24 hours. The treatment method reduces the severity of illness, compared to placebo, as assessed by an area under the curve analysis of total symptom scores. The treatment method reduces the duration of return to normal activity, compared to placebo. The treatment method reduces the duration of return to normal health, compared to placebo. The treatment method reduces the duration of return to normal oral temperature, compared to placebo.

### Example 2

This is a double-blind randomized, placebo-controlled trial of ERC (commercially available as Rayaldee®) in COVID-19 patients.

30 μg ERC capsules in a 900 μg loading dose are administered on Day 1, followed by a maintenance dose of 60 μg per day for the subsequent 26 days (Days 2-27). Placebo is administered in a look-alike 900 μg loading dose on Day 1, followed by a look-alike maintenance dose of 60 μg per day for the subsequent 26 days (Days 2-27). ERC and placebo doses are administered orally at bedtime after fasting for at least 3 hours.

Each subject meets the following criteria to be enrolled and randomized into one of the two treatment groups of this study: 1) be 50-85 years of age; and, 2) confirmed to have SARS-CoV-2 infection within the last 3 days. Participants i) can be male or female, ii) must be willing to limit the use of vitamin D supplements except for normally fortified food products (e.g., milk) during the course of the 4-week study, iii) must demonstrate the ability to comply with all study requirements and iv) must be without any disease state or physical condition that might impair evaluation of safety or which, in the investigator's opinion, would interfere with study participation.

Subjects who meet any of the following criteria are excluded from the study: Participants i) who have been on glucocorticoid medications in the last six months, ii) have a history of hyperparathyroidism, kidney stones, hypercalciuria or hypercalcemia, iii) have a history of a chronic granuloma-forming disease (e.g., sarcoidosis), iv) have any surgical or medical condition which might significantly alter the absorption, distribution, metabolism, or excretion of vitamin D (cholecalciferol or ergocalciferol) or 25-hydroxyvitamin D (e.g., small bowel resection), v) have ongoing treatment with thiazides or high doses of diuretics, vi) have renal impairment measured as eGFR<60 mL/min/1.73 m² on screening, vii) show evidence of existing or impending dehydration, viii) are known or suspected to have hypersensitivity to any of the constituents of the study drug, or ix) are currently participating in, or have participated in an investigational study within 30 days prior to study screening.

Concomitant Medications are allowed, with the exception of: (1) thiazides or high doses of non-thiazide diuretics; (2) medications that could impair the absorption of fat-soluble nutrients; and, (3) dietary supplements providing in excess of 1.0 g of elemental calcium per day.

Approximately 83 subjects are enrolled in each of the ERC treatment and placebo groups, for a total of approximately 166 subjects.

The study follows the following sequence of procedures.

Screening visit/Day 1—A brief clinical examination is completed (including height, weight and body mass index). Demographic information, health history, use of medications, any adverse events and the type and severity of COVID-19 symptoms (fever, cough, shortness of breath, difficulty breathing, chills, myalgia, headache, sore throat, and loss of taste or smell) using a 4-point NRS (0, absent; 3, severe) and oral temperature are recorded. Blood samples are obtained for determining serum parameters of interest. Randomization occurs during this visit for patients who meet the subject selection criteria. Each enrolled subject is instructed to take 900 μg of ERC (30×30 μg capsules) or an equivalent number of placebo capsules in the fasting state at bedtime. Each enrolled subject receives two additional bottles (30×30 μg capsules) of ERC or placebo to take home and is instructed to take two capsules (60 μg) at bedtime for the next 26 days.

Visit at Day 7—A blood sample is obtained to determine serum total 25-hydroxyvitamin D, creatinine, calcium and phosphorus concentrations.

Visit at Day 14—Treatment-emergent adverse events (TEAEs) and use of concomitant medications are recorded.

Visit at Day 28—Treatment-emergent adverse events (TEAEs) and use of concomitant medications are recorded. Blood samples are obtained for determination serum parameters of interest.

On each day during the dosing period, at bedtime, subjects: (i) record their ability to perform usual activities on a diary card using an 11-point NRS (unable to perform normal activity, 0; fully able to perform normal activity, 10); and, (ii) complete a record of their opinion of overall health status using an 11-point NRS (0, worst health and 10, best possible health).

A primary outcome measure is severity and duration of COVID-19 illness as evidenced by COVID-19 symptoms (fever, cough, shortness of breath, difficulty breathing, chills, myalgia, headache, sore throat, and loss of taste or smell) using a 4-point NRS (0, absent; 3, severe) and oral temperature recorded twice daily. A secondary outcome measure is attainment and maintenance of serum total 25-hydroxyvitamin D level at or above 50 ng/mL.

The primary efficacy endpoint is the duration of illness, defined as the time from study drug initiation to time of alleviation of symptoms. Symptom alleviation is considered to occur at the start of the first 24-hour period in which all recorded COVID-19 symptoms (fever, cough, shortness of breath, difficulty breathing, chills, myalgia, headache, sore throat, and loss of taste or smell) are scored 1 or less (mild or none) and remain so for that entire period. Subjects record twice daily the severity of each COVID-19 symptom using a 4-point NRS (0, absent; 3, severe) and their oral temperature using a digital thermometer.

Secondary outcome measures are (1) mortality rate; (2) incidence and duration of hospitalization; (3) requirement for mechanical ventilation; (4) number of subjects with at least one severe adverse event (SAE); (5) serum biomarkers of an innate and adaptive immune response; (6) severity and duration of COVID-19 illness as evidenced by quality-of-life measures including time to return to normal states of health and activity; (7) clinical evolution between Day 0 and Day 14 based on the change of symptom score for COVID-19, depending on serum total 25-hydroxyvitamin D concentration achieved at Day 7 (25D<30 ng/mL or ≥30 ng/mL); (8) clinical evolution between Day 0 and Day 28 based on the change of symptom score for COVID-19, depending on serum total 25-hydroxyvitamin D concentration achieved at Day 7 (25D<50 ng/mL or ≥50 ng/mL); (9) clinical evolution between Day 0 and Day 14 based on the change of symptom score for COVID-19, in patients with severe hypovitaminosis D (serum total 25-hydroxyvitamin D<20 ng/mL) at Day 0, depending on serum total 25-hydroxyvitamin D concentration achieved at Day 7 (25D<30 ng/mL or ≥30 ng/mL or 25D<50 ng/mL and ≥50 ng/mL); (10) clinical evolution between Day 0 and Day 28 based on the change of symptom score for COVID-19, in patients with severe hypovitaminosis D (serum total 25-hydroxyvitamin D<20 ng/mL) at Day 0, depending on serum total 25-hydroxyvitamin D concentration achieved at Day 7 (25D<50 ng/mL or ≥50 ng/mL).

Exploratory objectives include: (1) acute, subacute and late inflammation-response-related methylome and transcriptome in peripheral blood mononuclear cells (PBMCs); (2) serum total 1,25-dihydroxyvitamin D; (3) serum interleukin 1-β (IL-1β); (4) serum caspase-3.

Safety evaluations include changes in changes in: (1) serum calcium (corrected for serum albumin; see below); (2) serum phosphorus; and, (3) estimated glomerular filtration rate (eGFR).

Any subject who exhibits serum calcium >10.3 mg/dL, e.g. based on a blood sample obtained on Day 1 or 7, is removed from the study and the randomization status disclosed to the responsible investigator(s). Dosing is suspended based on a blood sample at Day 7 if serum total 25-hydroxyvitamin D exceeds 100 ng/mL.

Blood samples and serum anti-SARS-CoV-2 antibody titer is acquired on Days 1, 7 and 28. Blood samples are assayed for: (1) serum total 25-hydroxyvitamin D (primary efficacy measure), (2) serum total 1,25-dihydroxyvitamin D, (3) serum LL37, (4) serum interleukin-1β (IL-1β), caspase-3 and interleukin-6 (IL-6) as markers of the adaptive immune response to SARS-CoV-2 infection.

Treatment group subjects show a significant elevation of serum total 25-hydroxyvitamin D level compared to the placebo group, e.g. to at least >50 ng/mL. Treatment group subjects show significant quantitative reduction in viral titer from initial testing (on Day 7, 14, or 28), compared to the placebo group. Treatment group subjects also show significantly accelerated and quantitative increase in serum anti-SARS-CoV-2 antibodies (on Day 7, 14, or 28), compared to the placebo group. Treatment group subjects also show significantly reduced severity in one or more of the COVID-19 symptoms recorded on (on Day 7, 14, or 28), compared to the placebo group. Treatment group subjects also show significantly reduced duration in return to score 1 or less in one or more of the COVID-19 symptoms recorded on (on Day 7, 14, or 28), compared to the placebo group. Treatment group patients show acceleration of the acute, subacute, and late inflammation-response-related methylome and transcriptome in peripheral blood mononuclear cells compared to placebo group subjects.

The course of the disease is attenuated. Compared to placebo, hospital admissions for COVID-19 are significantly decreased in the treatment group. Compared to placebo, mortality due to COVID-19 is significantly decreased in the treatment group. Compared to placebo, requirement for mechanical ventilation is significantly decreased in the treatment group. Compared to placebo, SAEs are significantly decreased in the treatment group. Compared to placebo, viral titers are more rapidly decreased (on Day 7, 14, or 28) in the treatment group. The treatment method reduces (compared to placebo) time to resolution of illness, defined as time from study drug initiation to time of alleviation of symptoms, among individuals with SARS-COV-2 infection. Symptom alleviation is considered to occur at the start of the first 24-hour period in which all six symptoms are scored 1 or less (mild or none) and remain so for 24 hours. The treatment method reduces the severity of illness, compared to placebo, as assessed by an area under the curve analysis of total symptom scores. The treatment method reduces the duration of return to normal health, compared to placebo. The treatment method reduces the duration of return to normal oral temperature, compared to placebo. The treatment method reduces the duration of return to normal activity, compared to placebo.

### Example 3

This is a double-blind randomized, placebo-controlled trial of ERC (commercially available as Rayaldee®) in COVID-19 patients.

The primary objectives are to evaluate the effects of ERC vs. placebo treatment in patients with mild to moderate COVID-19 on the:
1) severity of disease as evidenced by eight COVID-19 symptoms (fever, cough, sore throat, malaise, headache, muscle pain, gastrointestinal symptoms and shortness of breath with exertion) using a 4-point NRS (absent, 0; severe, 3); and,
2) attainment and maintenance of serum total 25D levels consistently at or above 50 ng/mL.

The secondary objectives are to evaluate the effects of ERC vs. placebo treatment on:
1) duration of disease as evidenced by eight COVID-19 symptoms (fever, cough, sore throat, malaise, headache, muscle pain, gastrointestinal symptoms and shortness of breath with exertion) using a 4-point NRS (absent, 0; severe, 3);
2) mortality rates;
3) incidence and duration of hospitalizations;
4) incidence and duration of emergency room visits;
5) requirement for mechanical ventilation;
6) number of subjects with at least one severe adverse event (SAE);
7) severity and duration of COVID-19 illness as evidenced by quality-of-life measures including ability to perform usual activities and opinion of overall health status, using 11-point NRSs (unable to perform normal activity, 0; fully able to perform normal activity, 10) and (worst health, 0; best possible health, 10), respectively;
8) clinical evolution between Day 1 and Day 42 based on the change of symptom score for COVID-19, depending on serum 25D concentrations achieved at Days 7, 14, 21 and 28 (25D<30 ng/mL or ≥30 ng/mL);
9) clinical evolution between Day 1 and Day 42 based on the change of symptom score for COVID-19, depending on serum 25D concentrations achieved at Days 7, 14, 21 and 28 (25D<50 ng/mL or ≥50 ng/mL); and,
10) clinical evolution between Day 1 and Day 42 based on the change of symptom score for COVID-19, in patients with severe hypovitaminosis D (serum 25D<20 ng/mL) at Day 0, depending on serum 25D concentrations achieved at Days 7, 14, 21 and 28 (25D<30 ng/mL or ≥30 ng/mL or 25D<50 ng/mL or ≥50 ng/mL).

Exploratory objectives include evaluating the effects of ERC vs. placebo treatment on the:
1) acute, subacute and late inflammation-response-related change in the methylome and transcriptome of host peripheral blood mononuclear cells (PBMCs);
2) serum calcifediol (25D₃);
3) serum total 1,25D;
4) serum LL37;
5) serum intact parathyroid hormone (iPTH);
6) serum IL-1β;
7) serum caspase-3,
8) serum IL-6; and,
9) change in body weight.

Approximately 83 subjects are randomized to each of the ERC and placebo treatment groups, for a total of 166 subjects. The majority of subjects in each arm have stage 3 or 4 CKD, defined as an estimated glomerular filtration rate (eGFR) of 15 to <60 mL/min/1.73 m².

Subjects participate in the study for up to 45 days, up to 3 days for screening and randomization, 28 days of treatment, and 14 days of follow up (FU), once enrolled and randomized.

Efficacy evaluations of ERC vs. placebo-treated subjects include:
1) scored severity and duration of illness (see below);
2) attainment and maintenance of serum total 25D levels at ≥50 ng/mL;
3) mortality rates;
4) incidence and duration of hospitalization;
5) incidence and duration of emergency room visits;
6) requirement for mechanical ventilation;
7) changes in acute, subacute and late inflammation-response-related methylome and transcriptome of host PBMCs;
8) changes in serum 25D₃;
9) changes in serum total 1,25D;
10) changes in serum IL-1;
11) changes in serum caspase-3;
12) changes in serum IL-6; and,
13) changes in body weight.

The primary efficacy endpoint is severity of illness, defined as the total symptom score from study drug initiation (Day 1) to the end of study (Day 42). A statistically significant difference (P<0.05) between the treatment groups in the mean total symptom score in favor of treatment with ERC is a successful outcome. A secondary efficacy endpoint is duration of illness, defined as the time from study drug initiation (Day 1) to the first day when a complete absence of all eight COVID-19 symptoms is observed.

On each day during the dosing period, participants: i) record twice daily (once in the morning when the subject wakes up and once in the evening before the subject goes to sleep) the severity of eight COVID-19 symptoms (fever, cough, sore throat, malaise, headache, muscle pain, gastrointestinal symptoms and shortness of breath with exertion) using a 4-point NRS (absent, 0; severe, 3); ii) record once daily their ability to perform usual activities on a diary card using an 11-point NRS (unable to perform normal activity, 0; fully able to perform normal activity, 10); and, iii) complete once daily an assessment of overall health status using an 11-point NRS (worst health, 0 and best possible health, 10).

Safety is evaluated in all subjects, by changes in:
1) serum total calcium (corrected for low serum albumin; see below);
2) serum phosphorus; and,
3) eGFR.

Any subject who exhibits serum total calcium >10.5 mg/dL (corrected for serum albumin), based on blood samples obtained on Days 7, 14 and 21, reduces the daily maintenance dose from 60 μg to 30 μg until normalization of serum calcium at which time the dose is increased to 60 μg. The dose is similarly reduced to 30 μg per day based on blood samples at Days 7, 14 and 21 when serum total 25D exceeds 100 ng/mL. Dosing is suspended for any subject who exhibits serum total calcium >11.0 mg/dL (corrected for serum albumin), based on blood samples obtained on Days 7, 14 and 21, until normalization of serum calcium at which time dosing is resumed at 30 μg per day.

A schedule of events is summarized in the table below.

| | Screening and | | | | | | | | | | | |
|---|---|---|---|---|---|---|---|---|---|---|---|---|
| | Randomization | | Treatment Period | | | | | | | | Follow-up | |
| | (Day−3 to 1) | | (Days 2-28) | | | | | | | | (Days 29-42) | |
| | Visit | | | Visit | | Visit | | Visit | | Visit | | Visit |
| | 1 | | | 2 | | 3 | | 4 | | 5/ET | | 6 |
| | Days −3 | Day | | Day | | Day | | Day | | Day | | Day |
| | to 0 | 1 | Days | 7 | Days | 14 | Days | 21 | Days | 28 | Days | 42 |
| Study Procedure | Screening | Randomization | 2-6¹ | (±2) | 8-13¹ | (±2) | 15-20¹ | (±2) | 22-27¹ | (±2) | 29-41¹ | (±4) |
| Informed Consent | X | | | | | | | | | | | |
| Inclusion/exclusion criteria | X | | | | | | | | | | | |
| Medical history and | X | | | | | | | | | | | |
| demographics | | | | | | | | | | | | |
| Prior and concomitant | X | | | X | | X | | X | | X | | X |
| medications | | | | | | | | | | | | |
| Adverse events | | | | X | | X | | X | | X | | X |
| Physical examination | X | | | | | | | | | X | | |
| Vital signs² | X | | | X | | X | | X | | X | | |
| ECG³ | X | | | | | X | | | | X | | |
| Randomization | | X | | | | | | | | | | |
| Training on study | X | | | | | | | | | | | |
| drug administration | | | | | | | | | | | | |
| and diary reporting | | | | | | | | | | | | |
| Diary instructions | X | | | | | | | | | | | |
| dispensed | | | | | | | | | | | | |

¹All assessments performed by subject at home.
²Blood pressure, heart and respiratory rate, temperature and oxygen saturation (pulse oximetry) will be measured and recorded after the subject has been sitting for at least 2 minutes prior to any scheduled blood draws at every visit.
³Subject will be referred to a Cardiologist in the event of QT-prolongation.

| | Screening and | | | | | | | | | | | |
|---|---|---|---|---|---|---|---|---|---|---|---|---|
| | Randomization | | Treatment Period | | | | | | | | Follow-up | |
| | (Day−3 to 1) | | (Days 2-28) | | | | | | | | (Days 29-42) | |
| | Visit | | | Visit | | Visit | | Visit | | Visit | | Visit |
| | 1 | | | 2 | | 3 | | 4 | | 5/ET | | 6 |
| | Days −3 | Day | | Day | | Day | | Day | | Day | | Day |
| | to 0 | 1 | Days | 7 | Days | 14 | Days | 21 | Days | 28 | Days | 42 |
| Study Procedure | Screening | Randomization | 2-6¹ | (±2) | 8-13¹ | (±2) | 15-20¹ | (±2) | 22-27¹ | (±2) | 29-41¹ | (±4) |
| Study drug dispensed and | | X | | | | | | | | | | |
| shipped to subject's home | | | | | | | | | | | | |
| Study drug administration - | | X⁴ | X⁵ | | | | | | | | | |
| loading dose (self- | | | | | | | | | | | | |
| administered) | | | | | | | | | | | | |
| Study drug administration - | | | X⁷ | X | X | X | X | X | X | | | |
| maintenance dose (self- | | | | | | | | | | | | |
| administered)⁶ | | | | | | | | | | | | |
| Subject self-assessment for | X | X | X | X | X | X | X | X | X | X | X | X |
| COVID-19 symptom | | | | | | | | | | | | |
| severity (diary)⁸ | | | | | | | | | | | | |
| Subject self-assessment for | X | X | X | X | X | X | X | X | X | X | X | X |
| ability to perform normal | | | | | | | | | | | | |

⁴Study drug (30 capsules) will be administered at bedtime after fasting for 3 hours. Subject must continue to fast for 3 hours after administration of study drug.
⁵Days 2 and 3 only.
⁶Study drug (2 capsules) administered at bedtime after fasting for 3 hours. Subject must continue to fast for 3 hours after administration of study drug.
⁷Starting on Day 4.
⁸Completed at site during Screening and twice each day thereafter, once in the morning when the subject wakes up and once at bedtime (except Day 42 - once when subject wakes up).

| | Screening and | | | | | | | | | | | |
|---|---|---|---|---|---|---|---|---|---|---|---|---|
| | Randomization | | Treatment Period | | | | | | | | Follow-up | |
| | (Day−3 to 1) | | (Days 2-28) | | | | | | | | (Days 29-42) | |
| | Visit | | | Visit | | Visit | | Visit | | Visit | | Visit |
| | 1 | | | 2 | | 3 | | 4 | | 5/ET | | 6 |
| | Days −3 | Day | | Day | | Day | | Day | | Day | | Day |
| | to 0 | 1 | Days | 7 | Days | 14 | Days | 21 | Days | 28 | Days | 42 |
| Study Procedure | Screening | Randomization | 2-6¹ | (±2) | 8-13¹ | (±2) | 15-20¹ | (±2) | 22-27¹ | (±2) | 29-41¹ | (±4) |
| activities and overall health | | | | | | | | | | | | |
| status (diary)⁹ | | | | | | | | | | | | |
| Study drug administration | | X | X | X | X | X | X | X | X | X | | |
| recorded in diary | | | | | | | | | | | | |
| Study drug return and | | | | X | | X | | X | | X | | |
| compliance review | | | | | | | | | | | | |
| Diary review | | | | X | | X | | X | | X | | X |
| Laboratory Assessments | | | | | | | | | | | | |
| Hematology¹⁰ and serum | X | | | X | | X | | X | | X | | |
| chemistry¹¹ | | | | | | | | | | | | |
| Serum β-hCG¹² | X | | | X | | | | X | | X | | |
| Urine pregnancy test | | | | X | | X | | X | | X | | |
| Biomarkers, PD and PK | X | | | X | | X | | X | | X | | |
| parameters | | | | | | | | | | | | |

⁹Completed once each day at bedtime (except Day 42 - once when subject wakes up).
¹⁰Hematocrit, Hemoglobin, Platelet count, Red blood cell count, White blood cell count, Peripheral blood neutrophils, peripheral mononuclear monocytes.
¹¹Albumin, Alkaline phosphatase, Alanine aminotransferase (ALT), Aspartate aminotransferase (AST), Blood urea nitrogen, Total calcium (corrected), Carbon dioxide, Chloride, Creatinine, Estimated glomerular filtration rate (eGFR), Glucose, Lactate dehydrogenase, Magnesium, Phosphorus, Potassium, Sodium, Total bilirubin, Direct bilirubin, Total cholesterol, Total protein, Triglycerides, Uric acid.
¹²Performed at screening and at subsequent visits only following a positive urine pregnancy test result.

Blood samples are acquired periodically during the study, as described in the schedule above. Blood samples are assayed for: i) serum total 25D (primary efficacy measure) and 25D₃; ii) serum total 1,25D, iii) serum LL37, iv) serum iPTH, v) clinical chemistries and hematology, and vi) serum IL-1β, caspase-3 and IL-6 as markers of the adaptive immune response to SARS-CoV-2 infection. Harvested peripheral blood neutrophils and mononuclear monocytes are subjected to differential cell counting, immune cell surface marker phenotyping by fluorescent activated cell sorting (FACS) as well as differential whole genome bisulfite DNA sequencing with deconvolution analysis to ascertain the “signature” of unmethylated, transcriptionally-active genes in monocytes and neutrophils. Comparative pre- and serial post-intervention clinical disease severity and quality-of-life measures are assessed by daily scoring.

A 0.05 level two-sided log-rank test for equality of area-under-the-curve (AUC) has at least 80% power to detect a 25% difference in the total symptom score between Days 1 and 42 in the ERC treatment group and in the placebo group.

Baseline subject characteristics, including age, sex, race, ethnicity, height, weight, body mass index (BMI) and eGFR, are summarized by study arm (ERC and placebo). Primary, secondary and exploratory outcomes are summarized by study arm and follow-up time points, using CONSORT guidelines to present descriptive statistics, including mean, median, standard deviation (SD), quartiles, interquartile range, minimum, and maximum values for subjects within the site, overall and by treatment group. Continuous variables are summarized using means, SDs, and quartiles, while categorical variables are summarized using frequencies and percentages.

For the primary efficacy endpoint, a total COVID-19 symptom score is calculated for each person-day and an AUC is computed which is the sum of these scores over the 42-day duration of the study. A two sample t-test is used to compare the AUCs between study arms. A similar approach is used to evaluate the secondary efficacy endpoints related to quality of life self-assessments. The proportions of serum total 25D levels consistently ≥50 ng/mL are compared between the two treatment groups with Fisher's exact test. For the secondary efficacy endpoint of duration of disease, a two-sample log rank test is used to compare the mean endpoint of time (from Day 1) to complete symptom resolution between groups. Secondary analyses use Cox-proportional hazards regression models to adjust for covariates (e.g. age, sex, time from symptom onset to randomization). Analysis of changes from baseline in quantitative outcomes are performed using a linear mixed effects model, including a fixed study arm effect (ERC), a linear time effect (study arm by time interaction effect), baseline subject characteristics as covariates (e.g., age, sex, race, ethnicity, eGFR) and random subject effects to account for repeated measurements. Differences in changes between groups are estimated using model contrasts.

Each subject must meets the following criteria to be enrolled and randomized into one of the two treatment groups of this study:
1) male or female ≥18 years of age;
2) confirmed within the past 7 days to have SARS-CoV-2 infection as evidenced by a positive nasopharyngeal swab test using RT-PCR;
3) confirmed to have only mild or moderate SARS-CoV-2 infection based on presence of one or more of eight symptoms (fever, cough, sore throat, malaise, headache, muscle pain, gastrointestinal symptoms and shortness of breath with exertion) and the absence of clinical signs indicative of more severe disease;
4) willing to limit the use of vitamin D therapies or supplements except for normally fortified food products (e.g., milk) during the course of the 6-week study;
5) must demonstrate the ability to comply with all study requirements; and,
6) must be without any disease state or physical condition that might impair evaluation of safety or which, in the investigator's opinion, would interfere with study participation.

Subjects who meet any of the following criteria are excluded from the study:
1) clinical signs indicative of severe or critical COVID-19 severity;
2) pregnant or lactating women who are breastfeeding;
3) use of systemic glucocorticoid medications in the last six months;
4) recent history (previous 12 months) of primary hyperparathyroidism, kidney stones, hypercalciuria and/or hypercalcemia;
5) history of a chronic granuloma-forming disease (e.g., sarcoidosis);
6) history of tuberculosis or histoplasmosis;
7) history of chronic liver disease;
8) history (previous 12 months) of cardiac event indicative of chronic cardiovascular diseases including congestive heart failure, poorly controlled hypertension and arrhythmias;
9) history in the past five years of multiple myeloma or carcinoma of the breast, lung or prostate;
10) any surgical or medical condition which might significantly alter the absorption, distribution, metabolism, or excretion of vitamin D or 25-hydroxyvitamin D (e.g., small bowel resection, history of Crohn's Disease or Ulcerative Colitis);
11) ongoing treatment with thiazide diuretics;
12) history of hyperphosphatemia, hyperuricemia and gout;
13) renal impairment measured as eGFR<15 mL/min/1.73 m² on serum creatinine in the last three months;
14) serum calcium ≥9.8 mg/dL in the last three months;
15) evidence of existing or impending dehydration;
16) known or suspected to have hypersensitivity to any of the constituents of the study drug; and/or,
17) currently participating in, or have participated in, an interventional/investigational study within 30 days prior to study screening.

ERC capsules in a 900 μg loading dose split into three equal doses of 300 μg each is administered over three days (on Days 1, 2 and 3) followed by a maintenance dose of 60 μg per day for the subsequent 24 days (Days 4-27). Doses are administered orally at bedtime after fasting for at least 3 hours. Patients should remain fasted for at least 3 hours after administration of study drug.

The control product is placebo in a look-alike 900 μg loading dose split into three equal doses of 300 μg each administered over three days (on Days 1, 2, and 3) followed by a look-alike maintenance dose of 60 μg per day for the subsequent 24 days (Days 4-27). Doses are administered orally at bedtime after fasting for at least 3 hours. Patients should remain fasted for at least 3 hours after administration.

Concomitant medications are allowed, with the exception of:
1) calcitriol, paricalcitol and doxercalciferol;
2) thiazide diuretics;
3) medications that could impair the absorption of fat-soluble nutrients; and,
4) dietary supplements providing in excess of 1.0 g of elemental calcium per day.

Treatment group subjects show a significant elevation of serum total 25-hydroxyvitamin D level compared to the placebo group, e.g. to at least >50 ng/mL.

Compared to the placebo, group, treatment group subjects show improvement resulting from the treatment. Treatment group subjects show significant quantitative reduction in severity of disease as evidenced by eight COVID-19 symptoms (fever, cough, sore throat, malaise, headache, muscle pain, gastrointestinal symptoms and shortness of breath with exertion) using a 4-point NRS (absent, 0; severe, 3). Treatment group subjects show significantly reduced duration of disease as evidenced by eight COVID-19 symptoms (fever, cough, sore throat, malaise, headache, muscle pain, gastrointestinal symptoms and shortness of breath with exertion) using a 4-point NRS (absent, 0; severe, 3). Treatment group subjects show significant quantitative reduction in mortality rates. Treatment group subjects show significant quantitative reduction in incidence and duration of hospitalizations. Treatment group subjects show significant quantitative reduction in incidence and duration of emergency room visits. Treatment group subjects show significant quantitative reduction in requirement for mechanical ventilation. Treatment group subjects show significantly fewer number of subjects with at least one severe adverse event (SAE). Treatment group subjects show significant quantitative reduction in severity and duration of COVID-19 illness as evidenced by quality-of-life measures including ability to perform usual activities and opinion of overall health status, using 11-point NRSs (unable to perform normal activity, 0; fully able to perform normal activity, 10) and (worst health, 0; best possible health, 10), respectively. Treatment group subjects show significant quantitative reduction in clinical evolution between Day 1 and Day 42 based on the change of symptom score for COVID-19, depending on serum 25D concentrations achieved at Days 7, 14, 21 and 28 (25D<30 ng/mL or ≥30 ng/mL). Treatment group subjects show significant quantitative reduction in clinical evolution between Day 1 and Day 42 based on the change of symptom score for COVID-19, depending on serum 25D concentrations achieved at Days 7, 14, 21 and 28 (25D<50 ng/mL or ≥50 ng/mL). Treatment group subjects show significant quantitative reduction in clinical evolution between Day 1 and Day 42 based on the change of symptom score for COVID-19, in patients with severe hypovitaminosis D (serum 25D<20 ng/mL) at Day 0, depending on serum 25D concentrations achieved at Days 7, 14, 21 and 28 (25D<30 ng/mL or ≥30 ng/mL or 25D<50 ng/mL or ≥50 ng/mL). Treatment group subjects show acceleration of the acute, subacute, and late inflammation-response-related methylome and transcriptome in peripheral blood mononuclear cells compared to placebo group subjects.

### Example 4

A multi-center, double-blind randomized, placebo-controlled trial is ongoing to evaluate the safety and efficacy of ERC (commercially available as Rayaldee®) to treat symptomatic patients infected with SARS-CoV-2. The primary objectives are to evaluate the effects of ERC treatment vs. placebo in patients with mild to moderate COVID-19 on the:
1) severity and duration of disease as evidenced by COVID-19 symptoms using the InFLUenza Patient-Reported Outcome (FLU-PRO©) questionnaire*; and,
2) attainment and maintenance of serum total 25D levels at or above 50 ng/mL.
   Dr. John Powers, Leidos Biomedical and the National Institute for Allergy and Infectious Diseases (NIAID), National Institutes of Health.

The secondary objectives are to compare the effects of ERC treatment vs. placebo on:
1) incidence of emergency room/urgent care visits;
2) incidence of oxygen saturation below 94% (without supplemental oxygen);
3) incidence and duration of hospitalizations;
4) requirement for mechanical ventilation;
5) mortality rate;
6) number of subjects with at least one severe adverse event (SAE);
7) severity and duration of COVID-19 illness as evidenced by quality-of-life measures using the FLU-PRO©questionnaire; and,
7) clinical course of COVID-19 as a function of serum 25-hydroxyvitamin D concentrations of < or ≥30 ng/mL and < or ≥50 ng/mL at Days 7, 14, 21 and 28.

Exploratory objectives include evaluating the effects of ERC treatment vs. placebo on:
1) serum calcifediol (25-hydroxyvitamin D₃);
2) serum 24,25-dihydroxyvitamin D₃ (24,25D₃);
3) serum total 1,25-dihydroxyvitamin D;
4) serum LL37; 5) plasma intact parathyroid hormone (iPTH);
6) serum IL-1β;
7) serum caspase-1;
8) serum interleukin 6 (IL-6);
9) changes in DNA and RNA sequencing; and,
10) changes in body weight.

Approximately 80 subjects are being randomized to each of the ERC treatment and placebo groups, for a total of 160 subjects. Subjects are being randomized to receive either oral ERC or matching placebo administered according to the following regimen: a loading dose (900 mcg split into three equal doses of 300 mcg each administered on Days 1, 2 and 3) followed by 24 daily maintenance doses (60 mcg on Days 4 to 27) administered at bedtime after fasting for at least 3 hours following dinner. Patients remain fasted for at least 3 hours after administration of study drug. Concomitant medications are allowed, with the exception of: calcitriol, paricalcitol, doxercalciferol, thiazide diuretics, medications that could impair the absorption of fat-soluble nutrients, and dietary supplements providing in excess of 0.55 g of elemental calcium per day.

Subjects participate in the study for up to 45 days, up to 3 days for screening and randomization, 28 days of treatment, and 14 days of follow up (FU), once enrolled and randomized.

The primary efficacy endpoints are:
(1) the resolution of symptoms, defined as a reduction in the mean total FLU-PRO© symptom score at study drug initiation (Day 1) to or below 0.5 for a minimum of three consecutive days. The time to resolution is defined as the number of days from Day 1 to the first day of achieving the 0.5 score for a minimum of three consecutive days. A statistically significant difference (P<0.025 one sided) between the treatment groups in the time to resolution of symptoms in favor of treatment with ERC is deemed a successful outcome.
(2) attainment and maintenance of serum total 25D levels at or above 50 ng/mL, assessed at Days 21 and 28, where success is defined as levels ≥50 ng/mL on both of these days.

On each day during the study, subjects are instructed to record once daily (in the evening before the subject goes to sleep) the severity of COVID-19 symptoms using the FLU-PRO© questionnaire. This questionnaire includes quality of life measures such ability to perform usual activities and opinion of overall health status, using FLU-PRO©.

The safety and tolerability of Rayaldee® extended release capsules are being evaluated by adverse events (AEs), physical examinations (PE), vital signs (VS), electrocardiograms (ECGs), hematology and clinical chemistries. Special attention is given to changes in the following parameters:
(1) serum total calcium (corrected for serum albumin);
(2) serum phosphorus; and,
(3) estimated glomerular filtration rate (eGFR).
   The schedule of events for the study is summarized in the table below.

| | Screening and | | | | | | | | | | | |
|---|---|---|---|---|---|---|---|---|---|---|---|---|
| | Randomization | | | | | | | | | | | |
| | | Treatment Period | | | | | | | | | | |
| | | (Days 1-28) | | | | | | | | | Follow-up | |
| | | | | | | | | | | | (Days 29-42) | |
| | Visit 1 | | | Visit | | Visit | | Visit | | Visit | | Visit |
| | | Day 1 | | 2 | | 3 | | 4 | | 5/ET | | 6a |
| | Days −3 | Randomization | | Day | | Day | | Day | | Day | | Day |
| | to 0 | and initiation | Days | 7 | Days | 14 | Days | 21 | Days | 28 | Days | 42 |
| Study Procedure | Screening | of treatment | 2-6^{b} | (±2) | 8-13^{b} | (±2) | 15-20^{b} | (±2) | 22-27^{b} | (+2) | 29-41^{b} | (±4) |
| Informed Consent | X | | | | | | | | | | | |
| Inclusion/exclusion criteria | X | | | | | | | | | | | |
| Medical history and | X | | | | | | | | | | | |
| demographics | | | | | | | | | | | | |
| Prior and concomitant | X | | | X | | X | | X | | X | | X |
| medications | | | | | | | | | | | | |
| Adverse events | | | | X | | X | | X | | X | | X |
| Physical examinations^{c} | X | | | | | | | | | X | | |
| Vital signs^{d} | X | | | X | | X | | X | | X | | |
| ECG^{e} | X | | | | | X | | | | X | | |
| Randomization^{f} | | X | | | | | | | | | | |
| Training on study drug | X | | | | | | | | | | | |
| administration and diary | | | | | | | | | | | | |
| reporting | | | | | | | | | | | | |
| Diary instructions dispensed | X | | | | | | | | | | | |

aVisit is performed by telephone.
^{b}All assessments are performed by subject at home.
^{c}Brief physical exam.
^{d}Blood pressure, heart and respiratory rate, temperature and oxygen saturation (pulse oximetry) are measured and recorded after the subject has been sitting for at least 2 minutes prior to any scheduled blood draws at every visit. Height, weight and BMI at screening; weight and BMI at Visit 5.
^{e}Subject is referred to a Cardiologist in the event of QT-prolongation.
^{f}Study drug is shipped to the patient's home after confirmation of randomization. Date of receipt is Day 1 (first dose).

| | Screening and | | | | | | | | | | | |
|---|---|---|---|---|---|---|---|---|---|---|---|---|
| | Randomization | | | | | | | | | | | |
| | | Treatment Period | | | | | | | | | | |
| | | (Days 1-28) | | | | | | | | | Follow-up | |
| | | | | | | | | | | | (Days 29-42) | |
| | Visit 1 | | | Visit | | Visit | | Visit | | Visit | | Visit |
| | | Day 1 | | 2 | | 3 | | 4 | | 5/ET | | 6a |
| | Days −3 | Randomization | | Day | | Day | | Day | | Day | | Day |
| | to 0 | and initiation | Days | 7 | Days | 14 | Days | 21 | Days | 28 | Days | 42 |
| Study Procedure | Screening | of treatment | 2-6^{b} | (±2) | 8-13^{b} | (±2) | 15-20^{b} | (±2) | 22-27^{b} | (+2) | 29-41^{b} | (±4) |
| Study drug dispensed and | | X | | | | | | | | | | |
| shipped to subject's home | | | | | | | | | | | | |
| Study drug administration - | | x^{g} | x^{h} | | | | | | | | | |
| loading dose | | | | | | | | | | | | |
| (self-administered) | | | | | | | | | | | | |
| Study drug administration - | | | xⁱ | xⁱ | xⁱ | xⁱ | xⁱ | xⁱ | xⁱ | | | |
| maintenance dose (self- | | | | | | | | | | | | |
| administered) | | | | | | | | | | | | |
| Subject self-assessment for | X | X | X | X | X | X | X | X | X | X | X | X |
| severity of COVID-19 | | | | | | | | | | | | |
| symptoms and QOL using | | | | | | | | | | | | |
| the FLU-PRO ® | | | | | | | | | | | | |
| questionnaire (diary)^{j} | | | | | | | | | | | | |
| Study drug administration | | X | X | X | X | X | X | X | X | | | |
| recorded in diary | | | | | | | | | | | | |
| Study drug return and | | | | X | | X | | X | | X | | |
| compliance review | | | | | | | | | | | | |
| Diary review | | | | X | | X | | X | | X | | X |

^{g}Study drug (10 capsules) is administered at bedtime after fasting for 3 hours. Subject continues to fast for 3 hours after administration of study drug.
^{h}Days 2 and 3 only. Study drug (10 capsules on each day) is administered at bedtime after fasting for 3 hours. Subject continues to fast for 3 hours after administration of study drug.
ⁱMaintenance dose starting on Day 4 through Day 27 (2 capsules, unless otherwise directed, administered at bedtime after fasting for 3 hours. Subject continues to fast for 3 hours after administration of study drug).
^{j}Completes at Screening visit on site and thereafter once each day at bedtime (except Day 42 - once when subject wakes up).

| | Screening and | | | | | | | | | | | |
|---|---|---|---|---|---|---|---|---|---|---|---|---|
| | Randomization | | | | | | | | | | | |
| | | Treatment Period | | | | | | | | | Follow-up | |
| | | (Days 1-28) | | | | | | | | | (Days 29-42) | |
| | Visit 1 | | | Visit | | Visit | | Visit | | Visit | | Visit |
| | | Day 1 | | 2 | | 3 | | 4 | | 5/ET | | 6a |
| | | Randomization | | Day | | Day | | Day | | Day | | Day |
| | Days −3 to 0 | and initiation | Days | 7 | Days | 14 | Days | 21 | Days | 28 | Days | 42 |
| Study Procedure | Screening | of treatment | 2-6^{b} | (±2) | 8-13^{b} | (±2) | 15-20^{b} | (±2) | 22-27^{b} | (+2) | 29-41^{b} | (±4) |
| Laboratory Assessments | | | | | | | | | | | | |
| Hematology^{k} and serum^{l} | X | | | X | | X | | X | | X | | |
| chemistry | | | | | | | | | | | | |
| Serum β-hCG^{m} | X | | | | | X | | X | | X | | |
| Urine pregnancy test | | | | X | | X | | X | | X | | |
| Biomarkers, PD and PK | X | | | X | | X | | X | | X | | |
| parametersⁿ | | | | | | | | | | | | |

^{k}Hematocrit, Hemoglobin, Platelet count, Red blood cell count, White blood cell count, Peripheral blood neutrophils, peripheral mononuclear monocytes.
^{l}Albumin, Alkaline phosphatase, Alanine aminotransferase (ALT), Aspartate aminotransferase (AST), Blood urea nitrogen, Total calcium (corrected), Carbon dioxide, Chloride, Creatinine, Estimated glomerular filtration rate (eGFR) (calculated), Glucose, Lactate dehydrogenase, Magnesium, Phosphorus, Potassium, Sodium, Total bilirubin, Direct bilirubin, Total cholesterol, Total protein, Triglycerides, Uric acid.
^{m}Performed at screening and at subsequent visits only following a positive urine pregnancy test result.
ⁿSerum total 1, 25D, calcifediol, 24, 25D3, LL37, IL-1β, caspase-1 and IL-6; plasma iPTH and serum total 25D; PBMCs (for changes in DNA and RNA sequencing).

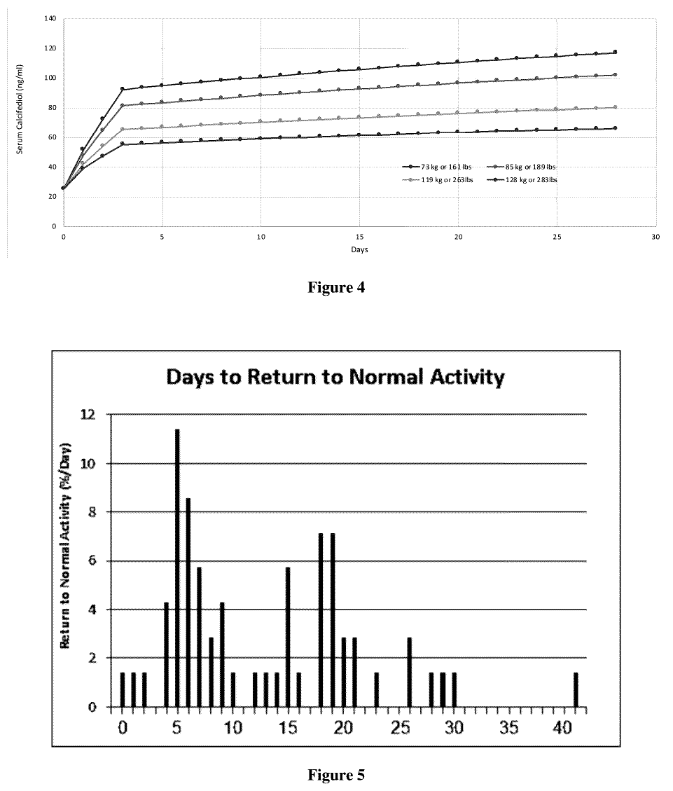

FIG. 2 shows projected serum 25-hydroxyvitamin D₃ levels in the treatment group, for subjects who dose in the fasted state according to the protocol, FIG. 3 shows projections for subjects who dose with food, and FIG. 4 shows projections for an intermediate combination thereof, modeled based on serum responses to Rayaldee® administration known to the present inventors. In each of these figures, the plot lines correspond, in top-to-bottom order, to patients having body weight of 73 kg, 85 kg, 119 kg, and 128 kg.

Any subject who exhibits a confirmed serum total calcium >10.5 and ≤11.0 mg/dL (corrected for serum albumin) based on blood samples obtained on Days 7 and 14 reduces the daily maintenance dose by one capsule (from 60 mcg to 30 mcg) starting at Day 21. The dose is similarly reduced based on blood samples obtained on Days 7 and 14 if serum total 25D is confirmed to exceed 100 ng/mL, starting at Day 21.

Subjects who exhibit confirmed serum total calcium >11.0 mg/dL (corrected for serum albumin) based on blood samples obtained on Days 7, 14 and 21 suspend dosing until normalization of serum calcium at which time (if applicable) dosing is resumed at one capsule per day (30 mcg per day). Subjects are requested to return to the clinic on the earliest possible day of their next scheduled visit (e.g., Day 12 for the Day 14 visit) for the confirmatory blood draw.

Subjects who develop severe (CTCAE≥3) abnormalities (low or high) of serum phosphorus, uric acid or plasma iPTH are withdrawn from study drug treatment.

Baseline subject characteristics, including age, sex, race, ethnicity, height, weight, body mass index (BMI) and eGFR, will be summarized by study arm (ERC and placebo). Primary, secondary and exploratory outcomes will be summarized by study arm and follow-up time points, using CONSORT guidelines to present descriptive statistics, including mean, median, standard deviation (SD), quartiles, interquartile range, minimum, and maximum values for subjects within the site, overall and by treatment group. Continuous variables will be summarized using means, SDs, and quartiles, while categorical variables are summarized using frequencies and percentages.

The two primary endpoints will be tested hierarchically to maintain an overall one-sided alpha level of 0.025. Therefore, the test of the attainment of serum 25D levels ≥50 ng/mL will be performed only if the resolution of symptoms is significant at <0.025.

The primary efficacy endpoint is the resolution of symptoms, defined as a reduction in the mean total FLU-PRO© symptom score at study drug initiation (Day 1) to or below 0.5 for a minimum of three consecutive days. The time to resolution is defined as the number of days from Day 1 to the first day of achieving the 0.5 score for a minimum of three consecutive days. The time to resolution will be presented as Kaplan-Meier curves and compared using a log-rank test. Subjects who die, are hospitalized or otherwise unable to report symptom scores due to illness will be considered as not having resolved, and will not be censored.

Attainment and maintenance of serum 25D levels at ≥50 ng/mL will be assessed with Chi-square statistics at Days 21 and 28, where success will be defined as levels ≥50 ng/mL on both of these days.

A Cox proportional hazards model will also be used to evaluate covariates.

Daily assessments of disease severity and quality-of-life measures are recorded by each subject using the FLU-PRO© questionnaire. In addition, each day subjects are asked to respond (yes/no) to whether they had returned to usual activities and usual health; to rate their physical health (5 point scale); for severity of infection symptoms (5 point scale); for how much their infection symptoms interfere with usual activities (5 point scale); and how their infection symptoms compare to those of the previous day (7 point scale). To further assess symptoms associated with COVID-19, subjects are asked daily whether they experienced loss of smell (anosmia), and loss of taste (ageusia) in the past day.

Each subject meets the following criteria to be enrolled and randomized into one of the two treatment groups of this study:
1) male or female ≥18 years of age;
2) confirmed within the past 3 days to have SARS-CoV-2 infection as evidenced by a positive nasopharyngeal swab test using RT-PCR;
3) confirmed to have only mild or moderate COVID-19 based on a FLU-PRO© score of at least 1.5 for each of the chest/respiratory and body/systemic domains, and the absence of clinical signs indicative of more severe disease (eg, oxygen saturation <94% on room air or respiration rate >30 bpm);
4) represents on self-assessment that the current COVID-19 symptoms are not consistent with usual health and that they are the same or worse than on the previous day;
5) willing to limit the use of vitamin D therapies or supplements except for normally fortified food products (eg, milk) during the course of the 6-week study;
6) must demonstrate the ability to comply with all study requirements; and,
7) must be without any disease state or physical condition that might impair evaluation of safety or which, in the investigator's opinion, would interfere with study participation.

Subjects who meet any of the following criteria are excluded from the study:
1) clinical signs indicative of severe or critical COVID-19 disease (eg, oxygen saturation <94% on room air or respiration rate >30 bpm);
2) pregnant or lactating women who are breastfeeding;
3) use of systemic glucocorticoid medications in the last six months;
4) recent history (previous 12 months) of primary hyperparathyroidism, kidney stones, hypercalciuria and/or hypercalcemia;
5) history of a chronic granuloma-forming disease (eg, sarcoidosis);
6) history of tuberculosis or histoplasmosis;
7) history of chronic liver disease;
8) history (previous 12 months) of cardiac event indicative of chronic cardiovascular diseases including congestive heart failure, poorly controlled hypertension and arrhythmias;
9) history in the past five years of multiple myeloma or carcinoma of the breast, lung or prostate;
10) any surgical or medical condition which might significantly alter the absorption, distribution, metabolism, or excretion of vitamin D or 25-hydroxyvitamin D (eg, small bowel resection, history of Crohn's disease or ulcerative colitis);
11) ongoing treatment with thiazide diuretics;
12) history of hyperphosphatemia, hyperuricemia and gout;
13) renal impairment measured as eGFR<15 mL/min/1.73 m² on serum creatinine in the last three months;
14) serum calcium ≥9.8 mg/dL in the last three months;
15) evidence of existing or impending dehydration;
16) known or suspected to have hypersensitivity to any of the constituents of the study drug; and/or,
17) currently participating in, or have participated in, an interventional/investigational study within 30 days prior to study screening

The FLU-PRO© questionnaire [Powers et al, BMC Infect Dis 2016; 16: 1 and Value in Health, Vol. 21, Issue 2, 2018, p. 210-218] is used by the enrolled subjects to self-assess their COVID-19 symptoms on a daily basis at bedtime. Mean symptom scores are calculated per symptom, per domain, and as a total score. See Powers et al, Value in Health, Vol. 21, Issue 2, 2018, p. 210-218. The daily diary also includes the additional questions described above (loss of taste and smell, and quality of life questions).

Subjects are instructed to take a loading dose of 10 capsules (300 mcg) of study drug per day on Days 1, 2 and 3 at bedtime after fasting for at least 3 hours following dinner, with any non-alcoholic liquid, by the oral route. On Days 4-27, subjects are instructed to take a maintenance dose of 2 capsules per day at bedtime unless otherwise directed. Patients are also instructed that they should remain fasted for at least 3 hours after administration of study drug.

Subjects are willing to limit the use of vitamin D therapy and vitamin D supplements except for normally fortified food products (e.g., milk) during the course of the 6-week study other than the study drug. Standard of care medications for CKD (vitamin D, calcitriol, paricalcitol and doxercalciferol) are being suspended during the treatment period. Excluded therapies at enrollment include thiazide diuretics, 1α-hydroxylated vitamin D analogs (calcitriol, paricalcitol and doxercalciferol) and vitamin D supplements (cholecalciferol and ergocalciferol).

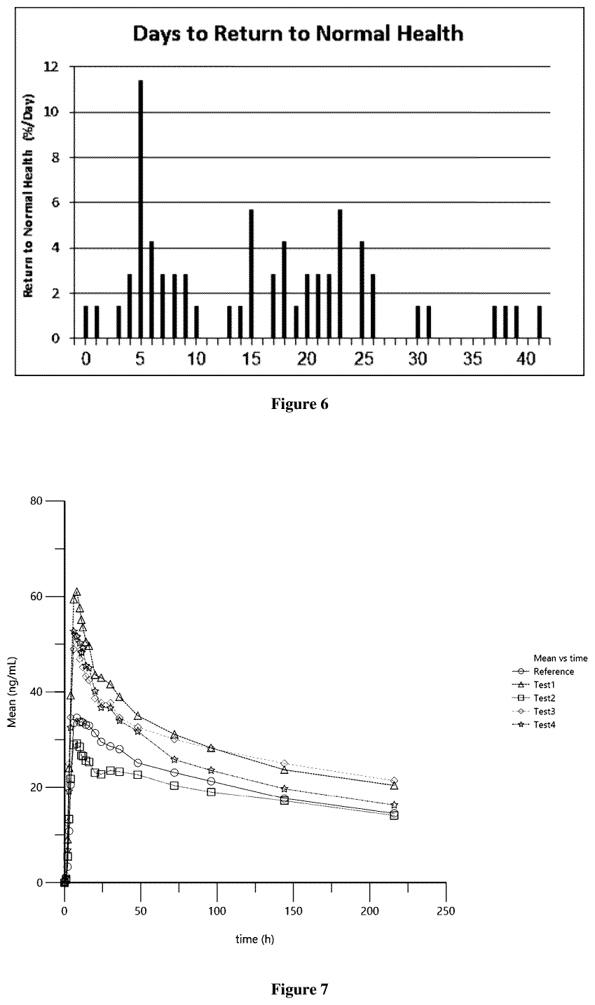

At this time, an analysis was performed blinded data for two of the quality of life measures assessed by the FLU-PRO© questionnaire on the first 70 subjects, namely the study day on which the subject self-reported a return to usual activities, and the study date on which the subject self-reported a return to usual health. A frequency distribution of these subjects who have returned to usual activities is displayed in FIG. 5. This frequency distribution shows that the subjects generally fall into two groups of approximately equal size: one which returns to usual activities earlier (peak frequency occurs after only 4-6 days) and one which returns to usual activities later (peak frequency occurs after 18-19 days). The slower return to usual activities in the latter group is consistent with similar data which have been published by Blair and co-workers for COVID-19 patients who are not treated with ERC. Blair et al. Open Forum Infect Dis. 2021 Jan. 5; 8(2):ofab007. A frequency distribution of study subjects who have returned to usual health is displayed in FIG. 6. This frequency distribution shows that the subjects also generally fall into two groups: one which returns to usual health earlier and one which returns to usual health later. The more rapid return to usual activities and health in the former groups suggests that ERC is effectively mitigating COVID-19 symptoms and fostering more rapid recovery.

It is contemplated that ERC treatment group subjects will show a significant elevation of serum total 25-hydroxyvitamin D level compared to the placebo group, e.g. to at least >50 ng/mL.

Compared to the placebo, group, it is contemplated that treatment group subjects will show one or more improvements resulting from the ERC treatment, as further described below. Treatment group subjects will show significant quantitative reduction in severity of disease as evidenced by COVID-19 symptoms using the FLU-PRO© questionnaire. Treatment group subjects will show significant quantitative reduction in duration of disease as evidenced by COVID-19 symptoms using the FLU-PRO© questionnaire.

Treatment group subjects will show significant quantitative reduction in incidence and duration of emergency room/urgent care visits. Treatment group subjects will show significant quantitative reduction in incidence of oxygen saturation below 94% (without supplemental oxygen). Treatment group subjects will show significant quantitative reduction in incidence of hospitalizations. Treatment group subjects will show significant quantitative reduction in duration of hospitalizations. Treatment group subjects will show significant quantitative reduction in requirement for mechanical ventilation. Treatment group subjects will show significant quantitative reduction in mortality rates. Treatment group subjects will show significant quantitative reduction in severity of COVID-19 illness as evidenced by quality-of-life measures using the FLU-PRO© questionnaire. Treatment group subjects will show significant quantitative reduction in duration of COVID-19 illness as evidenced by quality-of-life measures using the FLU-PRO© questionnaire.

Treatment group subjects will show significant quantitative reduction in severity and duration of COVID-19 illness as evidenced by reduction of mean symptom scores in one or more domains (nose, throat, eyes, chest/respiratory, gastrointestinal, and body/systemic) using the FLU-PRO© questionnaire. Treatment group subjects will show significant quantitative reduction in severity and duration of COVID-19 illness as evidenced by reduction of mean symptom scores in the nose domain using the FLU-PRO© questionnaire. Treatment group subjects will show significant quantitative reduction in severity and duration of COVID-19 illness as evidenced by reduction of mean symptom scores in the throat domain using the FLU-PRO© questionnaire. Treatment group subjects will show significant quantitative reduction in severity and duration of COVID-19 illness as evidenced by reduction of mean symptom scores in the eyes domain using the FLU-PRO© questionnaire. Treatment group subjects will show significant quantitative reduction in severity and duration of COVID-19 illness as evidenced by reduction of mean symptom scores in the chest/respiratory domain using the FLU-PRO© questionnaire. Treatment group subjects will show significant quantitative reduction in severity and duration of COVID-19 illness as evidenced by reduction of mean symptom scores in the gastrointestinal domain using the FLU-PRO© questionnaire. Treatment group subjects will show significant quantitative reduction in severity and duration of COVID-19 illness as evidenced by reduction of mean symptom scores in the body/systemic domain using the FLU-PRO© questionnaire.

Treatment group subjects will show significant quantitative reduction in clinical course of COVID-19 as a function of serum 25-hydroxyvitamin D concentrations of < or ≥30 ng/mL and < or ≥50 ng/mL at Days 7, 14, 21 and 28.

Treatment group subjects will show acceleration of the acute, subacute, and late inflammation-response-related methylome and transcriptome in peripheral blood mononuclear cells compared to placebo group subjects.

### Example 5

Provided in the table below are HPMC hard capsule formulations of 25-hydroxyvitamin D with varied percentages of paraffin wax and mineral oil (percentages by weight), and the related in vitro dissolution release rates.

| | 0% | 10% | 20% | 30% | 40% |
|---|---|---|---|---|---|
| | P. wax | P. wax | P. wax | P. wax | P. wax |
| Material | % Cap | % Cap | % Cap | % Cap | % Cap |
| calcifediol | 0.0176% | 0.0176% | 0.0176% | 0.0176% | 0.0176% |
| paraffin | 0.00% | 10.00% | 20.00% | 30.00% | 40.00% |
| mineral oil | 55.34% | 45.34% | 35.34% | 25.34% | 15.34% |
| hypromellose K100 | 10.00% | 10.00% | 10.00% | 10.00% | 10.00% |
| mono-and di-glycerides | 22.55% | 22.55% | 22.55% | 22.55% | 22.55% |
| lauroyl polyoxylglycerides | 9.75% | 9.75% | 9.75% | 9.75% | 9.75% |
| dehydrated ethanol | 2.32% | 2.32% | 2.32% | 2.32% | 2.32% |
| BHT | 0.02% | 0.02% | 0.02% | 0.02% | 0.02% |
| Total | 100.00% | 100.00% | 100.00% | 100.00% | 100.00% |

| | Soft Cap | 0% | 10% | 20% | 30% | 40% |
|---|---|---|---|---|---|---|
| Time (h) | 20% wax | Paraffin | Paraffin | Paraffin | Paraffin | Paraffin |
| 0 | 0.0 | 0.0 | 0.0 | 0.0 | 0.0 | 0.0 |
| 2 | 18.0 | 36.2 | 40.2 | 52.7 | 18.7 | 19.5 |
| 4 | 44.4 | 67.2 | 66.6 | 81.2 | 33.5 | 36.3 |
| 6 | 68.7 | 88.6 | 87.6 | 91.3 | 47.3 | 53.6 |
| 8 | 87.2 | 96.3 | 96.0 | 95.7 | 59.2 | 64.4 |
| 10 | 99.8 | 99.8 | 99.6 | 97.2 | 70.9 | 75.5 |
| 12 | 107.1 | 101.3 | 101.3 | 98.9 | 80.4 | 83.7 |

Reducing the paraffin wax below 20% (to 10% and 0%) with a related increase in mineral oil, compared to the 20 wt. % paraffin formulation, did not provide a significantly faster release profile compared to the comparative soft capsule formulation which contained 20% paraffin wax. On the other hand, increasing the paraffin wax above 20% to 30% and 40%, with a related decrease in mineral oil, did show considerable decrease in the in vitro release rates, especially after 2 hour time point.

### Example 6

The table below provides examples of additional wax-based hard capsule formulations, a Rayaldee-®-type soft capsule formulation (Reference) with a vegetable-based capsule shell, and modified wax-based soft vegetable-based capsule formulations modified with the goal of providing relatively slower and faster release compared to the Reference formulation. The soft capsules were OptiShell® vegetable-based capsules, containing modified starch and iota-carrageenan. The soft capsule fast (test 1) was formulated to give a fast release rate compared to the Reference by adjusting the concentration of the excipients. The soft capsule fast batch incorporated an increased amount of lauroyl polyoxylglycerides and a reduced amount of paraffin wax. Without intending to be bound by any particular theory, this modification of the matrix properties, to a less solid formulation and a higher concentration of the absorption enhancer compared to the Reference formulation, was intended to enhances the solubility of the active and, as a consequence, increase the release rate as well as the quantity absorbed in vivo, although it did not demonstrate a faster release rate in vitro. The table also includes pharmacokinetic profiles resulting from administering 900 μg doses to each of 16 adult subjects (extracted from mean baseline corrected serum concentration curves, FIG. 7).

| | | | Modified | Modified | | |
|---|---|---|---|---|---|---|
| | | Rayaldee ®- | wax-based | wax-based | HPMC | HPMC |
| | | type soft | soft | soft | Hard | Hard |
| | | capsule | capsule | capsule | capsule | capsule |
| | | (Reference) | (Slow) | (Fast) | (size 3) | (size 4) |
| Excipient | Function | Reference | Test 2 | Test 1 | Test 3 | Test 4 |
| calcifediol | 25-hydroxy- | 0.0176% | 0.0176% | 0.0176% | 0.0176% | 0.0194% |
| | vitamin | | | | | |
| | D active | | | | | |
| paraffin wax | control release | 20.00% | 39.00% | 5.00% | 28.00% | 19.95% |
| | agent | | | | | |
| mineral oil | carrier | 35.34% | 30.34% | 45.34% | 27.39% | 35.26% |
| hypromellose | stabilizer | 10.00% | 10.00% | 10.00% | 10.00% | 9.98% |
| mono & | emulsifier | 22.55% | 13.55% | 22.55% | 20.50% | 22.50% |
| diglycerides | | | | | | |
| lauroyl | absorption | 9.75% | 4.75% | 14.75% | 11.75% | 9.73% |
| polyoxyl | enhancer | | | | | |
| glycerides | | | | | | |
| dehydrated | solvent | 2.32% | 2.32% | 2.32% | 2.32% | 2.54% |
| ethanol | | | | | | |
| BHT | antioxidant | 0.02% | 0.02% | 0.02% | 0.02% | 0.02% |
| Total | — | 100.00% | 100.00% | 100.00% | 100.00% | 100.00% |
| Tmax (h) | | 8 | 8 | 8 | 8 | 6 |
| Cmax | | 34.58 | 61.03 | 29.21 | 51.65 | 52.69 |
| (ng/ml) | | | | | | |
| AUC | | 4536.51 | 6347.73 | 4081.29 | 6169.75 | 5385.26 |
| (ng · h · ml) | | | | | | |

FIG. 7 shows related mean serum concentration of 25-hydroxyvitamin D₃ curves after oral administration of 900 μg of the modified release calcifediol capsules. The increase of paraffin wax from 20% to 39% did slow the in vitro and in vivo release compared to the Reference, while the decrease of paraffin wax from 20% to 5% did not show fast release rate in vitro under the tested dissolution conditions. This result suggests that below 20% paraffin wax, an erosion mechanism may not be the predominant release mechanism for these formulations. The calcifediol in the fast and the Reference batches was solubilized to the same extent, and adding more emulsifier did not increase the solubility. Increasing the percentage of absorption enhancer from 9.75% to 14.75% had little effect in vitro under the conditions tested, while it increased the absorption in vivo, believed to be through other mechanisms, e.g. tissue penetration. The slow batch in the hard and soft capsules behaved as expected in vitro. The slow batch soft capsules also behaved as expected in vivo. The matrix in this batch is relatively rigid, and the erosion of the active from the rigid matrix is likely the predominant mechanism in this formulation.

The table blow provides dissolution time profiles for the formulations described above, according to USP Apparatus II (paddle with sinker).

| | Time | Time | Dissolution | STD | % |
|---|---|---|---|---|---|
| Formulation | (min) | (h) | (%) | (%) | RSD |
| Reference | 0 | 0 | 0 | 0 | 0 |
| Reference | 60 | 1 | 1.67 | 1.98 | 130.21 |
| Reference | 120 | 2 | 9.04 | 6.24 | 68.99 |
| Reference | 180 | 3 | 20.95 | 9.08 | 43.35 |
| Reference | 240 | 4 | 34.91 | 10.3 | 29.51 |
| Reference | 300 | 5 | 47.96 | 12.47 | 26 |
| Reference | 360 | 6 | 59.45 | 13.02 | 21.9 |
| Reference | 480 | 8 | 76.95 | 9.85 | 12.8 |
| Reference | 600 | 10 | 89.44 | 6.71 | 7.5 |
| Reference | 720 | 12 | 97.1 | 3.87 | 3.98 |
| Test1 | 0 | 0 | 0 | 0 | 0 |
| Test1 | 60 | 1 | 0.275 | 0.52 | 188.652 |
| Test1 | 120 | 2 | 7.564 | 3.089 | 40.833 |
| Test1 | 180 | 3 | 20.331 | 5.441 | 26.764 |
| Test1 | 240 | 4 | 34.255 | 6.303 | 18.4 |
| Test1 | 300 | 5 | 46.652 | 7.61 | 16.312 |
| Test1 | 360 | 6 | 59.015 | 8.374 | 14.19 |
| Test1 | 480 | 8 | 79.576 | 7.948 | 9.988 |
| Test1 | 600 | 10 | 91.199 | 4.816 | 5.281 |
| Test1 | 720 | 12 | 96.886 | 2.22 | 2.291 |
| Test2 | 0 | 0 | 0 | 0 | 0 |
| Test2 | 60 | 1 | 0.933 | 1.518 | 162.768 |
| Test2 | 120 | 2 | 5.823 | 2.687 | 46.141 |
| Test2 | 180 | 3 | 12.406 | 2.572 | 20.728 |
| Test2 | 240 | 4 | 18.377 | 3.092 | 16.826 |
| Test2 | 300 | 5 | 24.532 | 3.394 | 13.835 |
| Test2 | 360 | 6 | 30.788 | 5.149 | 16.723 |
| Test2 | 480 | 8 | 43.173 | 5.999 | 13.895 |
| Test2 | 600 | 10 | 54.453 | 6.125 | 11.248 |
| Test2 | 720 | 12 | 64.16 | 5.505 | 8.58 |
| Test3 | 0 | 0 | 0 | 0 | 0 |
| Test3 | 60 | 1 | 12.028 | 2.806 | 23.331 |
| Test3 | 120 | 2 | 23.336 | 2.622 | 11.234 |
| Test3 | 180 | 3 | 36.166 | 4.912 | 13.581 |
| Test3 | 240 | 4 | 46.122 | 5.707 | 12.373 |
| Test3 | 300 | 5 | 56.73 | 9.602 | 16.927 |
| Test3 | 360 | 6 | 66.205 | 10.866 | 16.412 |
| Test3 | 480 | 8 | 80.356 | 11.287 | 14.046 |
| Test3 | 600 | 10 | 90.945 | 10.168 | 11.18 |
| Test3 | 720 | 12 | 96.979 | 7.576 | 7.812 |
| Test4 | 0 | 0 | 0 | 0 | 0 |
| Test4 | 60 | 1 | 15.107 | 3.614 | 23.92 |
| Test4 | 120 | 2 | 35.029 | 5.557 | 15.863 |
| Test4 | 180 | 3 | 53.623 | 6.598 | 12.304 |
| Test4 | 240 | 4 | 67.86 | 6.892 | 10.156 |
| Test4 | 300 | 5 | 77.733 | 6.345 | 8.162 |
| Test4 | 360 | 6 | 85.366 | 4.774 | 5.592 |
| Test4 | 480 | 8 | 95.639 | 1.746 | 1.826 |
| Test4 | 600 | 10 | 98.212 | 1.582 | 1.611 |
| Test4 | 720 | 12 | 98.653 | 1.876 | 1.902 |

### Example 7

Described in the table below is another hard capsule formulation for 25-hydroxyvitamin D, with a gelatinized HPMC capsule shell. Gellan gum is a hydrophilic polymer and has similar properties to carrageenan used in the vegetable capsule shells of the Reference soft capsule formulation. The gelatinized HPMC capsule has a slower rupture/disintegration time in the stomach than non-gelatinized HPMC capsules.

| Fill Material | % of fill by weight | mg/Cap |
|---|---|---|
| calcifediol | 0.0194% | 0.03 |
| paraffin | 27.95% | 43.32 |
| mineral oil | 32.26% | 50 |
| hypromellose k100 | 9.98% | 15.47 |
| mono-and di-glycerides | 17.5% | 27.13 |
| lauroyl polyoxylglycerides | 9.73% | 15.08 |
| dehydrated ethanol | 2.54% | 3.94 |
| BHT | 0.02% | 0.03 |
| total | 100% | 155 |
| Shell Material | % of shell by weight | mg/Cap |
| hypromellose | qsp100 | 35.283 |
| gellan gum | 5 | 1.9 |
| titanium dioxide | 2 | 0.76 |
| Organic colorant | 0.15 | 0.057 |
| Total | 100 | 38 |

Paraffin wax at a level of 27.95% wax was used instead of 20% as in the Reference soft capsule formulation described above, with slight changes to the mineral oil and mono- and di-glycerides concentrations. The matrix fill was reduced to 155 mg per capsule instead of 170 mg, and the composition was filled in size 4 gelatinized HPMC capsule shells.

The in vivo dissolution profiles (USP Apparatus II (Paddle with Sinker) at 75 RPM, with a medium of 0.5% SDS in 5 mM Sodium Dihydrogenphosphate Monohydrate, pH 6.8, 37±0.5° C., with a volume of 500 ml) for the gelatinized HPMC hard capsule formulation, the Reference soft capsule formulation, and the Test 4 formulation described above are shown in the table below.

| | | | | | Gelatinized HPMC | |
|---|---|---|---|---|---|---|
| | | | | | hard capsule | |
| | Size 4 HC | | Reference | | formulation | |
| Time | Mean % | % | Mean % | % | Mean % | % |
| (hrs) | released | RSD | released | RSD | released | RSD |
| 0 | 0 | N/A | 0.0 | N/A | 0.0 | N/A |
| 1 | 15.1 | 23.9 | | | 11.2 | 19.7 |
| 2 | 35.0 | 15.9 | 10.2 | 35.4 | 25.3 | 17.1 |
| 3 | 53.6 | 12.3 | 37.9 | | 39.2 | 12.7 |
| 4 | 67.9 | 10.2 | 37.9 | 16 | 53.1 | 14.1 |
| 5 | 77.7 | 8.2 | | | 64.3 | 15.4 |
| 6 | 85.4 | 5.7 | 65.8 | 9.7 | 74.3 | 13.5 |
| 8 | 95.6 | 1.8 | 86.1 | 6.4 | 89.0 | 11.8 |
| 10 | 98.2 | 1.6 | 98.5 | 3.2 | 96.3 | 7.7 |
| 12 | 98.7 | 1.9 | 103.7 | 1.3 | 99.4 | 5.2 |

Similarly, a modified in vitro dissolution method was used to measure capsule dissolution (2 capsules in the vessel, 900 ml media, and 60 RPM), the results being shown in the table below (average of 5 Reference batches)

| | Average of 5 batches | | | |
|---|---|---|---|---|
| | Reference capsules | | Gelatinized HMPC hard cap | |
| | Mean % | | Mean % | |
| Time (hrs) | released | % RSD | released | % RSD |
| 0 | 0.0 | N/A | 0.0 | N/A |
| 1 | | | 8.6 | 14.4 |
| 2 | 12.8 | 19.5 | 20.2 | 14.1 |
| 3 | | | | |
| 4 | 41.5 | 11.3 | 43.1 | 12.2 |
| 5 | | | | |
| 6 | 68.2 | 7.5 | 63.5 | 9.8 |
| 8 | 85.6 | 4.3 | 79.7 | 7.6 |
| 10 | 93.4 | 1.8 | 90.0 | 3.8 |
| 12 | 95.7 | 1.4 | 93.0 | 2.9 |
| 14 | | | 93.2 | 2.3 |

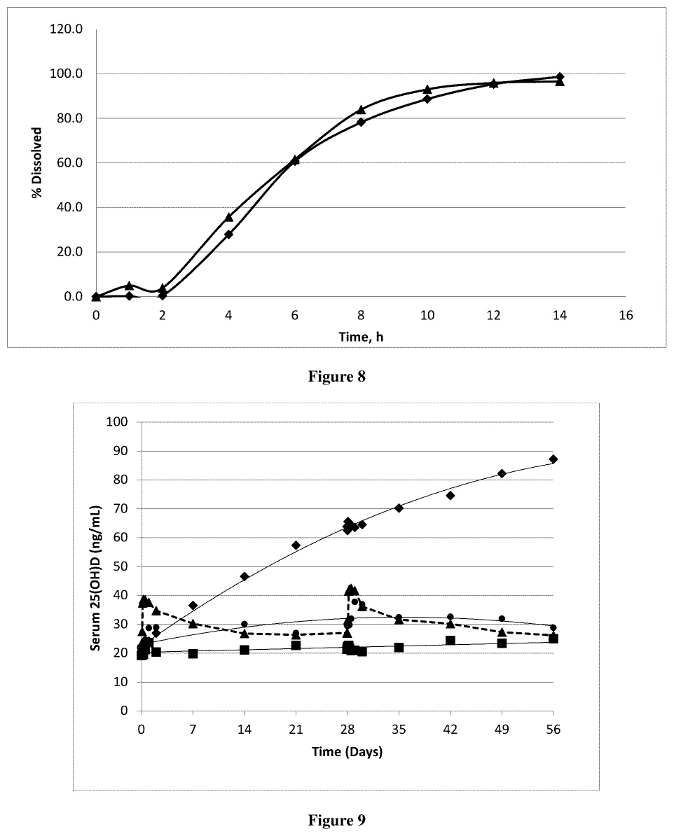

A two-stage dissolution method was also used (2 hours at pH 1.2, then transfer to pH 6.8) and the results are shown below and in FIG. 8 (average of 3 reference batches, diamond marker, versus gelatinized HPMC formulation, triangle marker). The dissolution profile of the gelatinized hard capsule formulation closely matches the dissolution profile of the vegetable-based soft capsule formulation.

| | | Average of 3 | gelatinized |
|---|---|---|---|
| | | Reference soft capsule | HPMC hard |
| | | batches | capsule |
| Time (hrs) | Media pH | % released | % released |
| 0 | pH 1.2 | 0.0 | 0 |
| 1 | | 0.3 | 5.0 |
| 2 | | 0.4 | 3.9 |
| 4 | pH 6.8 | 27.9 | 35.7 |
| 6 | | 60.7 | 61.6 |
| 8 | | 78.2 | 84.0 |
| 10 | | 88.7 | 93.0 |
| 12 | | 95.3 | 95.9 |
| 14 | | 98.6 | 96.6 |

The gelatinized HPMC hard capsule formulation and Reference soft capsule formulations are administered to subjects in the fasting state. The pharmacokinetic values and profiles resulting from administration (Cmax, AUC, Tmax) of the gelatinized HPMC hard capsule formulation more closely matches the values and profiles resulting from administration of the Reference formulation, compared to such values and profiles resulting from administration of non-gelatinized HPMC hard capsules, as described above.

### Example 8

A study is carried out with repeated-dosing of ERC (Rayaldee®-type formulation), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency.

This is an open-label study to gather comparative data evaluating the ERC, IR calcifediol, high-dose cholecalciferol, and paricalcitol plus low-dose cholecalciferol. Eligible subjects were randomized 1:1:1:1 to receive 8 weeks of treatment with one of the listed study medications with a sufficient quantity of a non-alcoholic beverage to enable swallowing of the capsules:
1) ERC capsules 60 μg once daily at bedtime, except on Days 1 and 29 when dosing occurred in the morning before breakfast in the phase 1 unit;
2) IR calcifediol 266 μg before breakfast on the mornings of Day 1 and Day 29 in the phase 1 unit;
3) cholecalciferol 300,000 IU (high-dose) before breakfast on the mornings of Day 1 and Day 29 in the phase 1 unit; and
4) paricalcitol 1 μg (possibly increasing to 2 μg per day at Day 29) plus cholecalciferol 800 IU (low-dose) once daily in the morning before breakfast, except on the mornings of Days 1 and 29 when dosing occurred before breakfast in the phase 1 unit. After 4 weeks of treatment, subjects who received paricalcitol doubled the dose to 2 μg plus cholecalciferol 800 IU once daily in the morning before breakfast provided that (a) the plasma iPTH did not decrease by at least 30% from pretreatment BL and remained above 70 μg/mL, (b) corrected serum calcium is <9.8 mg/dL, and (c) serum phosphorus is <5.5 mg/dL.

The subjects were housed in a phase 1 unit for approximately 14 to 26 hours at the beginning of the study and on study Day 29 to provide the blood samples required.

Subjects receiving treatment with calcitriol or other 1α-hydroxylated vitamin D analog or vitamin D supplementation prior to study completed a 4-week washout period prior to baseline (BL) assessments and remained off these non-study medications for the duration of the study. Subjects were excluded from enrollment if they have received calcimimetic therapy within 12 weeks preceding screening.

Blood samples were collected from all subjects at weekly intervals during the screening and BL periods and during the 8-week treatment period. Subjects maintained a dietary intake during the study of approximately 1,000-1,500 mg of elemental calcium per day by dietary counseling and, if necessary, a prescribed daily calcium supplement.

Subjects reduce the dose of study medication per the schedule below when plasma iPTH is confirmed to be <30 μg/mL, corrected serum calcium is confirmed to be >10.3 mg/dL, or serum phosphorus is confirmed to be >5.5 mg/dL. Subjects suspend dosing if plasma iPTH is confirmed to be <15 μg/mL or corrected serum calcium is confirmed to be >11.0 mg/dL, and resume dosing when plasma iPTH is ≥30 μg/mL and corrected serum calcium is <9.8 mg/dL per the dose schedule below.
ERC: decrease to 30 μg per day (from 60 μg per day)
IR calcifediol: hold Day 29 dose
Cholecalciferol 300,000 IU: hold Day 29 dose
Paricalcitol: decrease dose to 1 μg per day (from 2 μg per day)
Cholecalciferol 800 IU will not be adjusted
   In the event that a dose reduction is required for a subject receiving the minimum dosage of ERC (30 μg per day) or paricalcitol (1 μg per day), the subject will suspend dosing and resume when iPTH is ≥30 μg/mL and corrected serum calcium is <9.8 mg/dL at the same minimum dosage.

### Dose Resumption (if Needed):

ERC: 30 μg per day
Paricalcitol 1 μg per day

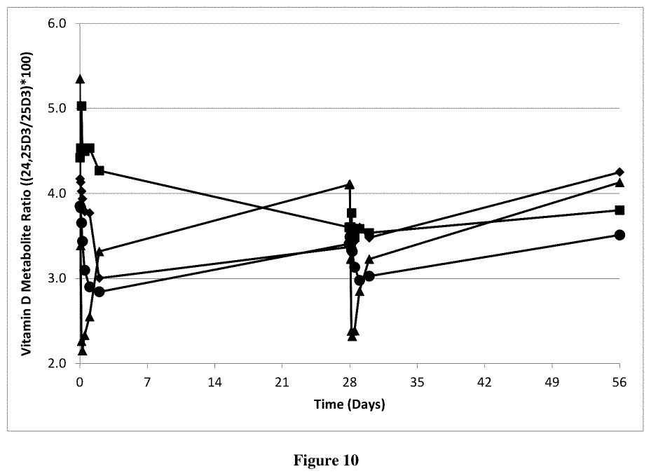

Mean serum total 25-hydroxyvitamin D concentrations as a function of time are shown in FIG. 9 (ERC group with diamonds, IR calcifediol group with triangles, cholecalciferol group with circles, and paricalcitol+cholecalciferol group with squares). The ERC group achieved serum concentrations greater than 50 ng/ml and nearly 90 ng/ml, while the VMR for that group remained below 5 (maximum of mean about 4.2 at end of treatment). VMR as a function of time is shown in FIG. 10.

The foregoing description is given for clearness of understanding only, and no unnecessary limitations should be understood therefrom, as modifications within the scope of the invention may be apparent to those having ordinary skill in the art.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Thus, throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure will control.

1. A method of treating a SARS-CoV-2 infection, comprising administering to a subject in need thereof an effective amount of a sustained release formulation of a 25-hydroxyvitamin D compound to treat a SARS-CoV-2 infection,
wherein an effective amount comprises administering to the subject a loading dose of the sustained release 25-hydroxyvitamin D formulation followed by one or more maintenance doses of the 25-hydroxyvitamin D compound, wherein the loading dose is greater than about 500 μg and less than about 1200 μg, and wherein the loading dose provides a serum total 25-hydroxyvitamin D level of at least 60 ng/mL and up to 200 ng/mL within 24 hours,
and wherein the subject's anti-SARS-CoV-2 antibody level is increased by Day 7 of treatment, compared to placebo.

2.-13. (canceled)

14. The method of claim 1, wherein the amount is effective to achieve a serum total 25-hydroxyvitamin D level of greater than 100 ng/mL in the subject.

15. The method of claim 1, wherein the serum total 25-hydroxyvitamin D level is maintained at an elevated level of greater than 60 ng/mL, for at least 7 days.

16. The method of claim 1, wherein the serum total 25-hydroxyvitamin D level is maintained at an elevated level of at least 60 ng/mL, for at least 14 days.

17.-19. (canceled)

20. The method of claim 1, wherein the loading dose is at least or about 900 μg of the 25-hydroxyvitamin D compound, and wherein the loading dose raises the subject's serum total 25-hydroxyvitamin D level by 20 ng/mL to 30 ng/mL within 10 hours.

21. The method of claim 20, wherein the loading dose is divided over two or three days.

22. (canceled)

23. The method of claim 21, wherein the loading dose is 300 μg per day for the first three days of treatment.

24. (canceled)

25. The method of claim 20, wherein the one or more maintenance doses is greater than about 50 μg of the 25-hydroxyvitamin D compound, and wherein the maintenance doses are administered on a frequency less than daily.

26. The method of claim 25, wherein the one or more maintenance doses are administered weekly, twice a week or three times a week.

27.-31. (canceled)

32. The method of claim 1, wherein the subject's vitamin D metabolite ratio (VMR, calculated as 100 times the ratio of serum 24,25-dihydroxyvitamin D₃ to serum 25-hydroxyvitamin D₃) is in a range of 4 to 12 in a first loading dose period and is in a range of 3 to 11 during a maintenance dosing period.

33. The method of claim 32, wherein the subject's VMR is less than 5, or less than 4.8 during a maintenance dosing period.

34. The method of claim 32, wherein the subject's VMR remains substantially constant or decreases over a period of at least 28 days, optionally during a maintenance dosing period.

35. The method of claim 32, wherein the subject is administered 25-hydroxyvitamin D by extended release, and the subject's rate of change of VMR over a 28 day period is less than the rate of change of VMR that would occur with a bioequivalent amount of the 25-hydroxyvitamin D administered by immediate release.

36.-41. (canceled)

42. The method of claim 1, wherein the 25-hydroxyvitamin D compound comprises 25-hydroxyvitamin D₂ or 25-hydroxyvitamin D₃, or a combination of 25-hydroxyvitamin D₂ and 25-hydroxyvitamin D₃.

43. The method of claim 42, wherein the 25-hydroxyvitamin D compound is 25-hydroxyvitamin D₃.

44.-103. (canceled)

104. The method of claim 1, wherein the loading dose of 25-hydroxyvitamin D is administered in a fasting state; and wherein the subject remains in a fasting state for three hours after the loading dose is administered.

105.-159. (canceled)

160. A dosing regimen for treating a patient having or suspected of having a SARS-CoV-2 infection, comprising administering to the patient an extended release dosage form comprising a controlled release excipient and a pharmaceutically effective amount of 25-hydroxyvitamin D₃ and wherein the dosage form is administered in a maintenance dosing period in an amount of 30 to 70 μg/day and preceded by a loading dosing period in an amount in a range of 300 to 900 μg/day on days one, two or three of treatment, wherein the patient's vitamin D metabolite ratio (VMR, calculated as 100 times the ratio of serum 24,25-dihydroxoxyvitamin D₃ to serum 25-hydroxyvitamin D₃) remains substantially constant or decreases during the maintenance dosing period.

161. The dosing regimen according to claim 160, wherein the patient's VMR is in a range of 4 to 12 in the first loading dose period and is in a range of 3-11 during the maintenance dosing period.

162. The dosing regimen according to claim 160, wherein the patient's rate of change of VMR over a 28 day period is less than the rate of change of VMR that would occur with a bioequivalent amount of the 25-hydroxyvitamin D administered by immediate release.

163. The dosing regimen according to claim 160, wherein the loading dose is administered to the patient subject in a fasting state; and wherein the patient remains in a fasting state for three hours after the loading dose is administered.


SEQUENCE LISTING (14 sequences)

SEQ ID NO: 1 - protein, 7096 aa, Severe acute respiratory syndrome-related coronavirus [sequence omitted]

SEQ ID NO: 2 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 222 aa, Severe acute respiratory syndrome-related coronavirus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, Severe acute respiratory syndrome-related coronavirus
MYSFVSEETGTLIVNSVLLFLAFVVFLLVTLAILTALRLCAYCCNIVNVSLVKPSFYVYSRVKNLNSSRVPDLLV

SEQ ID NO: 5 - protein, 419 aa, Severe acute respiratory syndrome-related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 7096 aa, Severe acute respiratory syndrome-related coronavirus
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNLAWRKAVFISPYNSQNAVASKILGLPTQTVDSSQGSECDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 7 - protein, 275 aa, Severe acute respiratory syndrome-related coronavirus
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 8 - protein, 75 aa, Severe acute respiratory syndrome-related coronavirus; identical to SEQ ID NO: 4

SEQ ID NO: 9 - protein, 222 aa, Severe acute respiratory syndrome-related coronavirus; identical to SEQ ID NO: 3

SEQ ID NO: 10 - protein, 61 aa, Severe acute respiratory syndrome-related coronavirus
MFHLVDFQVTIAEILLIIMRTFKVSIWNLDYIINLIIKNLSKSLTENKYSQLDEEQPMEID

SEQ ID NO: 11 - protein, 121 aa, Severe acute respiratory syndrome-related coronavirus
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFTTLCFTLKRKTE

SEQ ID NO: 12 - protein, 121 aa, Severe acute respiratory syndrome-related coronavirus
MKFLVFLGIITTVAAFHQECSLQSCTQHQPYVVDDPCPIHFYSKWYIRVGARKSAPLIELCVDEAGSKSPIQYIDIGNYTVSCSPFTINCQEPKLGSLVVRCSFYEDFLEYHDVRVVLDFI

SEQ ID NO: 13 - protein, 419 aa, Severe acute respiratory syndrome-related coronavirus; identical to SEQ ID NO: 5

SEQ ID NO: 14 - protein, 38 aa, Severe acute respiratory syndrome-related coronavirus
MGYINVFAFPFTIYSLLLCRMNSRNYIAQVDVVNFNLT